(19) United States
(12) Patent Application Publication
(10) Pub. No.: US 2007/0134690 A1
(43) Pub. Date: Jun. 14, 2007

(54) Diagnosis of systemic onset juvenile idiopathic arthritis through blood leukocyte microarray analysis

(75) Inventors: Maria Virginia Pascual, Dallas, TX (US); Jacques F. Banchereau, Dallas, TX (US); Damien Chaussabel, Richardson, TX (US); Florence Allantaz, Richardson, TX (US)

Correspondence Address: CHALKER FLORES, LLP, 2711 LBJ FRWY, Suite 1036, DALLAS, TX, 75234, US

(73) Assignee: Baylor Research Institute, Dallas, TX (US)

(21) Appl. No.: 11/544,377

(22) Filed: Oct. 6, 2006

Related U.S. Application Data
(60) Provisional application No. 60/724,526, filed on Oct. 7, 2005.

(51) Int. Cl. C12Q 1/68; G06F 19/00

(52) U.S. Cl. 435/6; 702/20

(57) ABSTRACT

The present invention includes compositions, systems and methods for the early detection and consistent determination of systemic onset juvenile idiopathic arthritis (SoJIA) using microarrays.

### CROSS-REFERENCE TO RELATED APPLICATIONS

This application claims priority to U.S. Provisional Application Ser. No. 60/724,526, filed Oct. 7, 2005, the contents of which is incorporated by reference herein in its entirety.

This invention was made with U.S. Government support under Contract No. National Institutes of Health R01 AR050770-01, CA78846 and U19A1057234-02. The government has certain rights in this invention. Without limiting the scope of the invention, its background is described in connection with diagnostic methods.

### TECHNICAL FIELD OF THE INVENTION

The present invention relates in general to the field of diagnostic for autoimmune diseases, and more particularly, to a system, method and apparatus for the diagnosis, prognosis and tracking of idiopathic systemic onset arthritis.

### BACKGROUND OF THE INVENTION

Juvenile idiopathic arthritis (JIA) is an important cause of short and long-term disability. The term JIA encompasses a heterogeneous group of diseases that are classified according to three major types of presentation: (i) oligoarthritis; (ii) polyarthritis; and (iii) systemic onset juvenile idiopathic arthritis (SoJIA). Each of these groups has different prognosis and responds differently to available therapies (1, 2), suggesting that their pathogenesis is also unique.

Children with SoJIA present with systemic symptoms, fever and/or rash, which precede the development of arthritis for weeks or even years. Once arthritis develops, these patients have a highly variable disease outcome. The overall prognosis correlates with the persistence of systemic symptoms and the number of joints involved six months after the initial presentation (3-6). Because of lack of success with conventional treatment, up to 50% of patients with SoJIA continue to have active arthritis 5-10 years after diagnosis (2, 7, 8). Since long term disability is directly correlated with duration of active disease, this group has the most severe outcome and thus has represented the most serious challenge to pediatric rheumatologists. The present inventors have recently shown that IL-1 is a major mediator of the inflammatory cascade underlying SoJIA (9). In fact, the present inventors found that IL-1Ra is an effective treatment for this disease (9-11).

One of the remaining challenges in managing patients with SoJIA is how to diagnose the disease at the time of presentation. As the symptoms (fever and/or rash) and the laboratory tests (anemia, leukocytosis, thrombocytosis and elevated erythrocyte sedimentation rate) are nonspecific, patients undergo extensive diagnostic tests and hospitalizations to exclude infections and malignancies. There is, therefore, a critical need to identify diagnostic markers.

### SUMMARY OF THE INVENTION

Systemic onset juvenile idiopathic arthritis (SoJIA) represents ˜10-20% of Juvenile Idiopathic Arthritis (JIA). The diagnosis of SoJIA relies on clinical findings as no specific diagnostic tests are available. The present inventors investigated the underlying immune dysregulation and found specific, reproducible blood leukocyte transcriptional signatures that permit, for the first time, the isolation and characterization of disease-specific diagnostic markers.

Gene-expression profiles were generated from peripheral blood samples obtained from 17 pediatric patients with SoJIA during the systemic phase of the disease. The average time from initiation of symptoms to diagnosis in these children was 6 months. These profiles were compared with those of 92 pediatric patients with acute infections caused by influenza A virus, gram-negative or gram-positive bacteria, 38 pediatric patients with Systemic Lupus Erythematosus (SLE) and 35 healthy controls.

Statistical group comparison and class prediction identified genes differentially expressed in SoJIA patients compared to healthy, which were, however, also changed in patients with acute infections and SLE. By performing a meta-analysis across all diagnostic groups, a list of 88 genes was identified. There 88 genes were specifically changed in patients with SoJIA. A subset of 12 genes that were part of this signature had the unique ability to identify patients with SoJIA. Importantly, the inventors found that this disease-specific signature was abrogated following the successful treatment of four patients with the IL-1 antagonist Anakinra. Therefore, the present invention may be used to detect and track disease, disease progression and the effectiveness of treatment.

It was also found that analysis of transcriptional signatures in blood leukocytes from SoJIA patients distinguishes SoJIA from other febrile illnesses caused by a variety of infectious agents. Availability of accurate diagnostic markers for SoJIA patients may allow prompt initiation of effective therapy and prevention of long-term disabilities.

The present invention includes a system and a method to analyze samples for the prognosis and diagnosis of Systemic Onset Juvenile Idiopathic Arthritis using multiple variable gene expression analysis. The gene expression differences that remain can be attributed with a high degree of confidence to the unmatched variation. The gene expression differences thus identified can be used, for example, to diagnose disease, identify physiological state, design drugs, and monitor therapies.

In one embodiment, the present invention includes a method of identifying a human subject predisposed to systemic onset juvenile idiopathic arthritis by determining the expression level of a biomarker comprising one or more of the following genes: delta hemoglobin; erythroid associated factor; Kruppel-like factor 1; myosin light polypeptide 4; and makorin 1; wherein the biomarker is correlated with a predisposition to systemic onset juvenile idiopathic arthritis. The biomarker may include transcriptional regulation genes selected from upregulation of Foxo3a, downregulation of GATA-3 and combinations thereof. Another example of biomarkers includes inflammatory/immune response genes selected from upregulation IL-1 receptor antagonist (IL-1RN), downregulation Fc Epsilon receptor and combinations thereof. A specific set of biomarkers mat be selected from the following:

| | | |
|---|---|---|
| 213415_at | CLIC2* | chloride intracellular channel 2 |
| 225352_at | TLOC1* | translocation protein 1 |
| 225394_s_at | MADP-1* | MADP-1 protein |
| 211994_at | —* | Clone A9A2BRB5 (CAC)n/(GTG)n |
| | | repeat-containing mRNA |
| 212055_at | C18orf10* | chromosome 18 open reading frame 10 |
| 212174_at | AK2* | adenylate kinase 2 |
| 228953_at | KIAA1971* | similar to junction-mediating and |
| | | regulatory protein p300 JMY |
| 230546_at | KIAA1036* | KIAA1036 |
| 230747_s_at | —* | CDNA clone IMAGE: 3029742, partial |
| | | cds |
| 242300_at | —* | — |
| 228622_s_at | DNAJC4* | DnaJ (Hsp40) homolog, subfamily C, |
| | | member 4 |
| 226296_s_at | MRPS15* | mitochondrial ribosomal protein S15 |

Additional examples of biomarkers include genes related to ubiquitination (solute carrier family 6/SLC6A8); components of the erythrocyte cytoskeleton (EBP42, tropomodulin 1); apoptosis (synuclein alpha) and combinations thereof. The biomarkers may be screened by quantitating the mRNA, protein or both mRNA and protein level of the biomarker. When the biomarker is mRNA level, it may be quantitated by a method selected from polymerase chain reaction, real time polymerase chain reaction, reverse transcriptase polymerase chain reaction, hybridization, probe hybridization, and gene expression array. The screening method may also include detection of polymorphisms in the biomarker. Alternatively, the screening step may be accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing. For use with the present invention the sample may be any of a number of immune cells, e.g., leukocytes or sub-components thereof.

Yet another embodiment of the present invention includes a computer implemented method for determining the genotype of a sample by obtaining a plurality of sample probe intensities; diagnosing systemic onset juvenile idiopathic arthritis based upon the sample probe intensities; and calculating linear correlation coefficient between the sample probe intensities and reference probe intensities; and accepting the tentative genotype as the genotype of the sample if the linear correlation coefficient is greater than a threshold value. In one embodiment, the threshold value is at least 0.8, 0.9, or even 0.95. In general, the probe intensities may be selected from a gene expression profile from the tissue sample where the expression profile of the two or more of the following genes is measured:

| | | |
|---|---|---|
| 213415_at | CLIC2* | chloride intracellular channel 2 |
| 225352_at | TLOC1* | translocation protein 1 |
| 225394_s_at | MADP-1* | MADP-1 protein |
| 211994_at | —* | Clone A9A2BRB5 (CAC)n/(GTG)n |
| | | repeat-containing mRNA |
| 212055_at | C18orf10* | chromosome 18 open reading frame 10 |
| 212174_at | AK2* | adenylate kinase 2 |
| 228953_at | KIAA1971* | similar to junction-mediating and |
| | | regulatory protein p300 JMY |
| 230546_at | KIAA1036* | KIAA1036 |
| 230747_s_at | —* | CDNA clone IMAGE: 3029742, partial |
| | | cds |
| 242300_at | —* | — |
| 228622_s_at | DNAJC4* | DnaJ (Hsp40) homolog, subfamily C, |
| | | member 4 |
| 226296_s_at | MRPS15* | mitochondrial ribosomal protein S15 |

as compared to a normal control sample.

Another embodiment includes a method for diagnosing systemic onset juvenile idiopathic arthritis from a tissue sample that includes obtaining a gene expression profile from the tissue sample wherein expression of the two or more of the following genes is measured:

| | | |
|---|---|---|
| 213415_at | CLIC2* | chloride intracellular channel 2 |
| 225352_at | TLOC1* | translocation protein 1 |
| 225394_s_at | MADP-1* | MADP-1 protein |
| 211994_at | —* | Clone A9A2BRB5 (CAC)n/(GTG)n |
| | | repeat-containing mRNA |
| 212055_at | C18orf10* | chromosome 18 open reading frame 10 |
| 212174_at | AK2* | adenylate kinase 2 |
| 228953_at | KIAA1971* | similar to junction-mediating and |
| | | regulatory protein p300 JMY |
| 230546_at | KIAA1036* | KIAA1036 |
| 230747_s_at | —* | CDNA clone IMAGE: 3029742, partial |
| | | cds |
| 242300_at | —* | — |
| 228622_s_at | DNAJC4* | DnaJ (Hsp40) homolog, subfamily C, |
| | | member 4 |
| 226296_s_at | MRPS15* | mitochondrial ribosomal protein S15 |

as compared to a normal control sample. The tissue used for the source of biomarker, e.g., RNA, may be a leukocyte.

Yet another embodiment of the present invention is a computer readable medium with computer-executable instructions for performing the method for determining the genotype of a sample by obtaining a plurality of sample probe intensities; diagnosing systemic onset juvenile idiopathic arthritis based upon the sample probe intensities for heme synthesis (delta hemoglobin or erythroid associated factor), erythrocyte-specific transcription factors (Kruppel-like factor 1), cytoskeleton (myosin light polypeptide 4), ubiquitin ligase (makorin 1), IL-1 receptor antagonist (IL-1RN), Fc Epsilon receptor, Foxo3a or GATA-3; and calculating a linear correlation coefficient between the sample probe intensities and reference probe intensities; and accepting the tentative genotype as the genotype of the sample if the linear correlation coefficient is greater than a threshold value. The threshold value may be at least about 0.8, 0.9 or 0.95 and the gene expression profile from a tissue sample may include two or more of the following genes:

| | | |
|---|---|---|
| 213415_at | CLIC2* | chloride intracellular channel 2 |
| 225352_at | TLOC1* | translocation protein 1 |
| 225394_s_at | MADP-1* | MADP-1 protein |
| 211994_at | —* | Clone A9A2BRB5 (CAC)n/(GTG)n |
| | | repeat-containing mRNA |
| 212055_at | C18orf10* | chromosome 18 open reading frame 10 |
| 212174_at | AK2* | adenylate kinase 2 |
| 228953_at | KIAA1971* | similar to junction-mediating and |
| | | regulatory protein p300 JMY |
| 230546_at | KIAA1036* | KIAA1036 |
| 230747_s_at | —* | CDNA clone IMAGE: 3029742, partial |
| | | cds |
| 242300_at | —* | — |
| 228622_s_at | DNAJC4* | DnaJ (Hsp40) homolog, subfamily C, |
| | | member 4 |
| 226296_s_at | MRPS15* | mitochondrial ribosomal protein S15 |

as compared to a normal control sample. In one embodiment, the number of genes selected for the analysis is 2, 3, 4, 5, 6, 7, 8, 9, 10, 11 or 12.

Yet another embodiment is a microarray for identifying a human subject predisposed to systemic onset juvenile idiopathic arthritis in which a microarray is used for the detection of gene expression, wherein the microarray includes four or more biomarker selected from the group consisting of delta hemoglobin; erythroid associated factor; Kruppel-like factor 1; myosin light polypeptide 4; makorin 1, IL-1 receptor antagonist (IL-1RN), Fc Epsilon receptor, Foxo3a, and GATA-3; wherein the gene expression data obtained from the microarray correlates to a predisposition to systemic onset juvenile idiopathic arthritis with a threshold value of at least 0.8.

The diagnosing systemic onset juvenile idiopathic arthritis may include obtaining gene expression data from a microarray and determining the expression four or more biomarkers selected from the group consisting of delta hemoglobin; erythroid associated factor; Kruppel-like factor 1; myosin light polypeptide 4; makorin 1, IL-1 receptor antagonist (IL-1RN), Fc Epsilon receptor, Foxo3a, and GATA-3; wherein the gene expression data obtained from the microarray correlates to a predisposition to systemic onset juvenile idiopathic arthritis with a threshold value of at least 0.8.

The method for diagnosing systemic onset juvenile idiopathic arthritis from a tissue sample may include obtaining a gene expression profile from the tissue sample wherein expression of the two or more of the following genes is measured:

| | | | | |
|---|---|---|---|---|
| | | | Average | |
| | | | normalized | |
| | Gene | | values in | |
| Probe Set ID | Symbol | p-value | SoJIA | Gene Title |

Microtubule/Cytoskeleton

| | | | | |
|---|---|---|---|---|
| 200703_at | DNCL1 | 2.16E−04 | 1.7 | dynein, cytoplasmic, light polypeptide 1 |
| 207490_at | TUBA4 | 3.96E−04 | 1.4 | tubulin, alpha 4 |

Extracellular matrix

| | | | | |
|---|---|---|---|---|
| 216993_s_at | COL11A2 | 0.00241 | 1.4 | collagen, type XI, alpha 2 |
| 202337_at | PMF1 | 9.06E−04 | 0.7 | polyamine-modulated factor 1 |

Ubiquitination

| | | | | |
|---|---|---|---|---|
| 200718_s_at | SKP1A | 0.00462 | 1.3 | S-phase kinase-associated protein 1A |
| | | | | (p19A) |
| 201824_at | RNF14 | 0.00301 | 2.0 | ring finger protein 14 |
| 210579_s_at | TRIM10 | 0.00835 | 1.4 | tripartite motif-containing 10 |

Transport

| | | | | |
|---|---|---|---|---|
| 201066_at | CYC1 | 5.27E−04 | 0.8 | cytochrome c-1 |
| 202125_s_at | ALS2CR3 | 5.27E−04 | 2.1 | amyotrophic lateral sclerosis 2 |
| | | | | (juvenile) chromosome region, |
| | | | | candidate 3 |
| 213415_at | CLIC2* | 1.69E−05 | 8.3 | chloride intracellular channel 2 |
| 215716_s_at | ATP2B1 | 0.00241 | 0.6 | ATPase, Ca++ transporting, plasma |
| | | | | membrane 1 |
| 218211_s_at | MLPH | 0.00462 | 1.5 | melanophilin |
| 224787_s_at | RAB18 | 6.94E−04 | 0.7 | RAB18, member RAS oncogene |
| | | | | family |
| 225352_at | TLOC1* | 1.10E−05 | 2.4 | translocation protein 1 |
| 226154_at | DNM1L | 0.00836 | 0.8 | Dynamin 1-like |
| 238066_at | RBP7 | 0.00836 | 0.8 | retinol binding protein 7, cellular |
| 244227_at | SYT6 | 0.00241 | 1.3 | synaptotagmin VI |

Apoptosis

| | | | | |
|---|---|---|---|---|
| 212373_at | FEM1B | 5.27E−04 | 0.7 | Fem-1 homolog b (C. elegans) |
| 235116_at | TRAF1 | 9.06E−04 | 1.3 | TNF receptor-associated factor 1 |

Metabolism

| | | | | |
|---|---|---|---|---|
| 209301_at | CA2 | 0.00374 | 2.6 | carbonic anhydrase II |
| 209509_s_at | DPAGT1 | 0.0015 | 1.2 | dolichyl-phosphate N- |
| | | | | acetylglucosaminephosphotransferase 1 |

Transcription

| | | | | |
|---|---|---|---|---|
| 202484_s_at | MBD2 | 0.00191 | 0.7 | methyl-CpG binding domain protein 2 |
| 224099_at | KCNH7 | 0.00191 | 1.5 | potassium voltage-gated channel, |
| | | | | subfamily H (eag-related), member 7 |
| 224933_s_at | JMJD1C | 0.00374 | 0.7 | jumonji domain containing 1C |
| 225527_at | CEBPG | 0.00117 | 0.7 | CCAAT/enhancer binding protein |
| | | | | (C/EBP), gamma |
| 227685_at | TMF1 | 0.0069 | 0.8 | TATA element modulatory factor 1 |
| 228785_at | ZNF281 | 0.00241 | 0.6 | Zinc finger protein 281 |
| 235389_at | PHF20 | 0.00462 | 0.8 | PHD finger protein 20 |
| 35671_at | GTF3C1 | 2.16E−04 | 1.3 | general transcription factor IIIC, |
| | | | | polypeptide 1, alpha 220 kDa |

Nuclear mRNA splicing, via spliceosome

| | | | | |
|---|---|---|---|---|
| 223416_at | SF3B14 | 0.00241 | 0.8 | splicing factor 3B, 14 kDa subunit |
| 225394_s_at | MADP-1* | 2.62E−06 | 0.6 | MADP-1 protein |

Glysocylation

| | | | | |
|---|---|---|---|---|
| 201724_s_at | GALNT1 | 0.00462 | 0.9 | UDP-N-acetyl-alpha-D- |
| | | | | galactosamine:polypeptide N- |
| | | | | acetylgalactosaminyltransferase 1 |
| 210205_at | B3GALT4 | 5.27E−04 | 1.3 | UDP-Gal: betaGlcNAc beta 1,3- |
| | | | | galactosyltransferase, polypeptide 4 |

Phosphorylation

| | | | | |
|---|---|---|---|---|
| 211992_at | WNK1 | 5.27E−04 | 2.1 | WNK lysine deficient protein kinase 1 |
| 226979_at | MAP3K2 | 0.00567 | 0.7 | Mitogen-activated protein kinase |
| | | | | kinase kinase 2 |
| 227073_at | MAP3K2 | 0.00836 | 0.8 | Mitogen-activated protein kinase |
| | | | | kinase kinase 2 |

Protein Biosynthesis

| | | | | |
|---|---|---|---|---|
| 212225_at | SUI1 | 2.16E−04 | 0.6 | Putative translation initiation factor |
| 224302_s_at | MRPS36 | 0.00374 | 0.8 | mitochondrial ribosomal protein S36 |
| 226296_s_at | MRPS15* | 3.80E−05 | 0.6 | mitochondrial ribosomal protein S15 |

Protein folding

| | | | | |
|---|---|---|---|---|
| 201759_at | TBCD | 1.12E−04 | 2.2 | tubulin-specific chaperone d |
| 225061_at | DNAJA4 | 0.00191 | 2.4 | DnaJ (Hsp40) homolog, subfamily A, |
| | | | | member 4 |
| 228622_s_at | DNAJC4* | 3.80E−05 | 0.7 | DnaJ (Hsp40) homolog, subfamily C, |
| | | | | member 4 |

Unknown

| | | | | |
|---|---|---|---|---|
| 211994_at | —* | 2.62E−06 | 2.8 | Clone A9A2BRB5 (CAC)n/(GTG)n |
| | | | | repeat-containing mRNA |
| 212055_at | C18orf10* | 5.54E−05 | 2.0 | chromosome 18 open reading frame |
| | | | | 10 |
| 212174_at | AK2* | 8.80E−07 | 0.7 | adenylate kinase 2 |
| 212341_at | MGC21416 | 0.00836 | 1.6 | hypothetical protein MGC21416 |
| 212829_at | — | 6.94E−04 | 2.0 | CDNA FLJ13267 fis, clone |
| | | | | OVARC1000964 |
| 216739_at | — | 3.96E−04 | 1.6 | — |
| 218116_at | C9orf78 | 0.00191 | 2.1 | chromosome 9 open reading frame 78 |
| 218126_at | FLJ10579 | 9.06E−04 | 1.5 | hypothetical protein FLJ10579 |
| 218583_s_at | RP42 | 0.00462 | 1.5 | RP42 homolog |
| 218936_s_at | HSPC128 | 0.00117 | 0.6 | HSPC128 protein |
| 222309_at | C6orf62 | 0.00567 | 0.6 | Chromosome 6 open reading frame |
| | | | | 62 |
| 223112_s_at | NDUFB10 | 3.96E−04 | 0.8 | NADH dehydrogenase (ubiquinone) 1 |
| | | | | beta subcomplex, 10, 22 kDa |
| 223548_at | C1orf26 | 0.0015 | 1.4 | chromosome 1 open reading frame 26 |
| 224807_at | KIAA1533 | 0.0015 | 0.8 | KIAA1533 |
| 224915_x_at | — | 9.06E−04 | 0.7 | Similar to RPE-spondin |
| 225202_at | RHOBTB3 | 0.0069 | 1.2 | Rho-related BTB domain containing 3 |
| 225213_at | TA-PP2C | 2.16E−04 | 0.8 | T-cell activation protein phosphatase |
| | | | | 2C |
| 225819_at | TBRG1 | 0.00241 | 0.7 | transforming growth factor beta |
| | | | | regulator 1 |
| 226833_at | FLJ32499 | 0.00301 | 1.3 | hypothetical protein FLJ32499 |
| 226927_at | — | 0.00374 | 1.2 | Homo sapiens, clone |
| | | | | IMAGE: 3894337, mRNA |
| 227265_at | — | 0.00301 | 0.8 | MRNA; cDNA DKFZp686N07104 |
| 228452_at | C17orf39 | 0.00625 | 1.6 | chromosome 17 open reading frame |
| | | | | 39 |
| 228953_at | KIAA1971* | 5.54E−05 | 0.6 | similar to junction-mediating and |
| | | | | regulatory protein p300 JMY |
| 229074_at | EHD4 | 0.00117 | 0.8 | EH-domain containing 4 |
| 229653_at | FLJ10979 | 0.00836 | 1.4 | Hypothetical protein FLJ10979 |
| 230118_at | — | 2.16E−04 | 1.3 | Transcribed locus |
| 230421_at | LOC345462 | 0.00567 | 1.2 | similar to hypothetical protein |
| | | | | 9630041N07 |
| 230546_at | KIAA1036* | 7.95E−05 | 1.6 | KIAA1036 |
| 230747_s_at | —* | 3.80E−05 | 0.7 | CDNA clone IMAGE: 3029742, partial |
| | | | | cds |
| 232486_at | LRFN1 | 0.00462 | 1.4 | leucine rich repeat and fibronectin |
| | | | | type III domain containing 1 |
| 232709_at | — | 0.00191 | 0.7 | CDNA FLJ13427 fis, clone |
| | | | | PLACE1002477 |
| 233469_at | psiTPTE22 | 0.00301 | 1.3 | TPTE pseudogene |
| 234305_s_at | MLZE | 9.06E−04 | 1.4 | melanoma-derived leucine zipper, |
| | | | | extra-nuclear factor |
| 235798_at | — | 0.00117 | 0.8 | — |
| 236196_at | — | 0.0015 | 0.7 | CDNA FLJ42548 fis, clone |
| | | | | BRACE3004996 |
| 241491_at | KIAA1002 | 6.94E−04 | 1.5 | KIAA1002 protein |
| 241517_at | — | 0.00117 | 1.3 | — |
| 241817_at | FLJ43654 | 3.96E−04 | 0.7 | FLJ43654 protein |
| 242003_at | LOC157697 | 0.00301 | 0.7 | Hypothetical protein LOC157697 |
| 242300_at | —* | 2.56E−05 | 4.0 | — |
| 243109_at | MCTP2 | 2.94E−04 | 1.7 | Multiple C2-domains with two |
| | | | | transmembrane regions 2 |
| 243434_at | FLJ10874 | 0.00836 | 1.2 | Hypothetical protein FLJ10874 |
| 244092_at | ZRANB3 | 0.0015 | 1.4 | Zinc finger, RAN-binding domain |
| | | | | containing 3 |
| 244390_at | — | 0.0015 | 1.8 | Transcribed locus |
| 244728_at | LOC130063 | 0.00462 | 1.4 | hypothetical gene LOC130063 |
| 53987_at | RANBP10 | 2.94E−04 | 1.8 | RAN binding protein 10 |

as compared to a control.

The present invention also includes a system for diagnosing systemic onset juvenile idiopathic arthritis by determining the expression level of four or more biomarkers selected from the group consisting of delta hemoglobin; erythroid associated factor; Kruppel-like factor 1; myosin light polypeptide 4; makorin 1, IL-1 receptor antagonist (IL-1RN), Fc Epsilon receptor, Foxo3a, and GATA-3; wherein the expression data obtained correlates to a predisposition to systemic onset juvenile idiopathic arthritis with a threshold value of at least 0.8. The expression level may be the measurement of protein levels.

### BRIEF DESCRIPTION OF THE DRAWINGS

For a more complete understanding of the features and advantages of the present invention, reference is now made to the detailed description of the invention along with the accompanying figures and in which:

FIG. 1A is a flowchart of the analysis scheme.

FIG. 1B shows the differential gene expression in PBMCs isolated from SoJIA patients and healthy controls. 17,454 genes passing the control criteria were tested. Genes expressed at statistically different levels between the 2 groups (p<0.01, Wilcoxon-Mann-Whitney test, Bonferroni correction) were rearranged by hierarchical clustering in order to reveal differential expression. Expression values are normalized per-gene to the healthy group. Transformed expression levels are indicated by color scale, with red representing relative high expression and blue indicating relative low expression. A list of the genes shown in this figure is available in Table IV.

FIG. 2A. Class prediction for 8 Healthy and 8 SOJIA samples obtained from the initial study group were used as a training set to generate a list of classifier genes displaying the best ability to discriminate patients from healthy controls. In this training set, 100% of patients were classified accurately.

FIG. 2B shows those classifier genes were then tested on a test set (8 Healthy and 9 SOJIA). In this test set, 100% of patients were classified accurately. Expression values were normalized per-gene to the healthy group. Samples and genes were arranged by hierarchical clustering. Transformed expression levels are indicated by color scale, with red representing relative high expression and blue indicating relative low expression. Transformed expression levels are indicated by color scale, with red representing relative high expression and blue indicating relative low expression. The list of the genes from this figure is shown in Table I.

FIG. 2C shows the validation of discriminative genes by real-time RT-PCR for the expression levels of 8 genes measured by real-time RT-PCR in five groups of patients: Healthy, SOJIA, S. aureus, S. pneumoniae, E. coli and Influenza A.

FIG. 2D summarizes the expression levels of the same 8 genes measured using microarrays. P-values were calculated between the healthy and SOJIA groups (Wilcoxon-Mann-Whitney test).

FIG. 3 shows the specificity of the SoJIA signature. The 50 best classifier genes from FIG. 2 were used to classify a test set of 35 healthy controls, 17 SoJIA, 31 S. aureus, 12 S. pneumoniae, 31 E. coli, 18 influenza A and 38 SLE patients. The number of samples within each disease group predicted as SoJIA is represented on top of the figure. Genes were arranged by hierarchical clustering. Transformed expression levels are indicated by color scale, with red representing relative high expression and blue relative low expression. * cross-validation.

FIGS. 4A and 4b show the SoJIA-specific signature. In FIG. 4A, genes expressed at statistically different levels in the SoJIA patients group compared to healthy volunteers (p<0.01, Wilcoxon-Mann-Whitney test) were selected (4311 probe sets). P-values were similarly obtained from patients suffering from S. aureus, E. coli, influenza A, S. pneumoniae and SLE. Each of these cohorts was compared to the appropriate control group. FIG. 4B shows that out of those 4311 genes, 88 were found expressed at statistically different levels in the SoJIA patients group compared to healthy controls (p<0.01, Wilcoxon-Mann-Whitney test) but not in all of the others groups (p<0.5, Wilcoxon-Mann-Whitney test). (C) The 12 most significant genes (p-value<0.0001 in SOJIA group) were used as predictors genes to predict a test set of 35 healthy, 17 SoJIA, 31 S. aureus, 12 S. pneumoniae, 31 E. coli, 18 influenza A and 38 SLE. P values are represented according to color scale: Turquoise=low p-value; Pink=High p-value. Expression values of those 12 genes were normalized per-gene to the healthy group. Genes were arranged by hierarchical clustering. Transformed expression levels are indicated by color scale, with red representing relative high expression and blue indicating relative low. expression. The list of the 88 genes shown in B and C are represented in Table II. * cross-validation.

FIGS. 5A and 5B show the effect of Anakinra on the specific SOJIA signature. Eighty eight genes from FIG. 4C were analyzed in FIG. 5A shows the expression profile of 4 patients before and 8 weeks after initiation of treatment with Anakinra. FIG. 5B shows the same patients on two occasions taken two years apart while the patient was active and not receiving Anakinra. Genes were arranged by hierarchical clustering. Normalized values in a healthy control are shown on the left column. Transformed expression levels are indicated by color scale, with red representing relative high expression and blue indicating relative low expression. The list of the genes shown in this figure is available in Table II.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “subject” refers to a human or other mammal. It is intended that the term encompasses healthy individuals, as well as, individuals predisposed to, or suspected of having a Juvenile Idiopathic Arthritis (JIA), e.g., a Systemic onset juvenile idiopathic arthritis (SoJIA). Typically, the terms “subject” and “patient” are used interchangeably.

The term “gene” refers to a nucleic acid (e.g., DNA) sequence that includes coding sequences necessary for the production of a polypeptide (e.g.,), precursor, or RNA (e.g., mRNA). The polypeptide may be encoded by a full length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The tnRNA functions during translation to specify the sequence or order of amino acids in a nascent polypeptide.

As used herein, the term “nucleic acid” refers. to any nucleic acid containing molecule, including but not limited to, DNA, cDNA and RNA. In particular, the terms “a gene in Table X” refers to at least a portion or the full-length sequence listed in a particular table, as found hereinbelow. The gene may even be found or detected a genomic form, that is, it includes one or more intron(s). Genomic forms of a gene may also include sequences located on both the 5′ and 3′ end of the coding sequences that are present on the RNA transcript. These sequences are referred to as “flanking” sequences or regions. The 5′ flanking region may contain regulatory sequences such as promoters and enhancers that control or influence the transcription of the gene. The 3′ flanking region may contain sequences that influence the transcription termination, post-transcriptional cleavage, MRNA stability and polyadenylation.

As used herein, the term “biomarker” refers to DNA, RNA or protein that is correlated with a particular condition. In some embodiments, the biomarker refers to a DNA, RNA or protein that is correlated with a predisposition to developing JIA or SoJIA.

The biomarker may be either a greater or lesser level of MRNA transcribed from a gene of interest, or a greater or lesser level of protein encoded by a gene of interest. The biomarker may even include one or more polymorphism(s) in a DNA, RNA and/or protein. Examples of biomarkers for use with the present invention include any one of the tables herein, e.g., probes to one or more of the following genes:

| Probe Set ID | Gene Symbol | Gene Title |
|---|---|---|
| 213415_at | CLIC2* | chloride intracellular channel 2 |
| 225352_at | TLOC1* | translocation protein 1 |
| 225394_s_at | MADP-1* | MADP-1 protein |
| 211994_at | —* | Clone A9A2BRB5 (CAC)n/(GTG)n |
| | | repeat-containing mRNA |
| 212055_at | C18orf10* | chromosome 18 open reading frame 10 |
| 212174_at | AK2* | adenylate kinase 2 |
| 228953_at | KIAA1971* | similar to junction-mediating and |
| | | regulatory protein p300 JMY |
| 230546_at | KIAA1036* | KIAA1036 |
| 230747_s_at | —* | CDNA clone IMAGE: 3029742, partial |
| | | cds |
| 242300_at | —* | — |
| 228622_s_at | DNAJC4* | DnaJ (Hsp40) homolog, subfamily C, |
| | | member 4 |
| 226296_s_at | MRPS15* | mitochondrial ribosomal protein S15 |

However, the present invention is not limited to this list of biomarkers. In fact additional suitable biomarkers are detected using the methods and compositions described herein.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “polymorphism” refers to the regular and simultaneous occurrence in a single interbreeding population of two or more alleles of a gene, where the frequency of the rarer alleles is greater than can be explained by recurrent mutation alone (typically greater than 1%).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide protein) chain. The DNA sequence thus codes for the amino acid sequence.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “Southern blot” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58, 1989).

As used herein, the term “Northern blot” refers to the analysis of RNA by electrophoresis of RNA on agarose gels, to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52, 1989).

As used herein, the term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. Any probe used in the present invention may be labeled with any “reporter molecule,” so that it is detectable in any detection system, including, but not limited to enzyme (e.g., ELISA, as well as enzyme-based histochemical assays), fluorescent, radioactive, luminescent systems and the like. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “real time PCR” as used herein, refers to various PCR applications in which amplification is measured during as opposed to after completion of the reaction. Reagents suitable for use in real time PCR embodiments of the present invention include but are not limited to TaqMan probes, molecular beacons, Scorpions primers or double-stranded DNA binding dyes.

As used-herein, the term “transcriptional upregulation” as used herein refers to an increase in synthesis of RNA, by RNA polymerases using a DNA template. For example, when used in reference to the methods of the present invention, the term “transcriptional upregulation” refers to an increase of least 2 fold, 2 to 3 fold, 3 to 10 fold, and even greater than 10 fold, in the quantity of mRNA corresponding to a gene of interest detected in a sample derived from an individual predisposed to JIA or SoJIA as compared to that detected in a sample derived from an individual who is not predisposed to JIA or SoJIA. Particularly useful differences are those that are statistically significant.

Conversely, the term “transcriptional downregulation” refers to a decrease in synthesis of RNA, by RNA polymerases using a DNA template. For example, when used in reference to the methods of the present invention, the term “transcriptional downregulation” refers to a decrease of least 2 fold, 2 to 3 fold, 3 to 10 fold, and even greater than 10 fold, in the quantity of mRNA corresponding to a gene of interest detected in a sample derived from an individual predisposed to JIA or SoJIA as compared to that detected in a sample derived from an individual who is not predisposed to such a condition or to a database of information for wild-type and/or normal control. Particularly useful differences are those that are statistically significant.

Both transcriptional “upregulation” and transcriptional “downregulation” may also be indirectly monitored through measurement of the translation product or protein level corresponding to the gene of interest. The present invention is not limited to any given mechanism related to upregulation or downregulation of transcription.

As used herein, the terms “array,” “chip,” “probe array,” and “microarray” refer to a small solid surface (e.g., glass) on which thousands of oligonucleotide or polynucleotide probes have been deposited (e.g., robotically) and immobilized in a predetermined order permitting automated recording of sample hybridization information. Some embodiments of the present invention comprise “GeneChip.RTM. expression arrays” (Affymetrix) for the qualitative and quantitative measurement of gene expression levels in a biologically relevant organism (e.g., human, rat, mouse, etc.).

The term “eukaryotic cell” as used herein refers to a cell or organism with membrane-bound, structurally discrete nucleus and other well-developed subcellular compartments. Eukaryotes include all organisms except viruses, bacteria, and bluegreen algae.

As used herein, the term “in vitro transcription” refers to a transcription reaction comprising a purified DNA template containing a promoter, ribonucleotide triphosphates, a buffer system that includes DTT and magnesium ions, and an appropriate RNA polymerase, which is performed outside of a living cell or organism.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the term “diagnosis” refers to the determination of the nature of a case of disease. In some embodiments of the present invention, methods for making a diagnosis are provided which permit determination of JIA or even SoJIA.

As used herein, an “expression profile” refers to the measurement of the relative abundance of a plurality of cellular constituents. Such measurements may include, RNA or protein abundances or activity levels. The expression profile can be a measurement for example of the transcriptional state or the translational state. See U.S. Pat. Nos. 6,040,138, 5,800,992, 6,020135, 6,033,860 and U.S. Ser. No. 09/341,302 which are hereby incorporated by reference in their entireties. The gene expression monitoring system, include nucleic acid probe arrays, membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are expressly incorporated herein by reference. The gene expression monitoring system may also comprise nucleic acid probes in solution.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue.

Differentially Expressed: The term differentially expressed as used herein means that the measurement of a cellular constituent varies in two or more samples. The cellular constituent can be either up-regulated in the test sample relative to the reference or down-7 regulated in the test sample relative to one or more references. Differential gene expression can also be used to distinguish between cell types or nucleic acids. See U.S. Pat. No. 5,800,992, relevant portions incorporated herein by reference.

Patient information. Blood samples were obtained from 17 patients with SoJIA during the systemic phase of the disease (median age: 5 years; range: 2-17 years), 29 patients with E. coli infection (7 years; 2 weeks-16 years), 31 patients with S. aureus infection (7 years; 3 months-18 years), 12 patients with S. pneumoniae (2.35 years; 3.3 months-16 years), 18 with Influenza A infections (1.5 years; 3 weeks-16 years), and 38 patients with SLE (12 years; 5-18). Patients were divided in training and test sets according to age and treatment (Table III). Subjects were recruited at Texas Scottish Rite Hospital (TSRH) and Children's Medical Center of Dallas (CMC). The study was approved by all the Institutional Review Boards and informed consent was obtained from all patients. Bacterial and viral infections were confirmed by standard bacterial cultures, direct fluorescent antigen testing and viral cultures. Patients with infections were recruited once a confirmed microbiologic diagnosis was established. Respiratory viral cultures were performed in 60 of 73 (82%) patients with bacterial infections. The clinical characteristics of these patients have been reported elsewhere (Ramilo, et al., submitted).

RNA and Microarray Sample Preparation. All blood samples were obtained in EDTA purple-top tubes (BD Vaccutainer). Fresh Peripheral Blood Mononuclear Cells (PBMCs) were isolated via Ficoll gradient. Cells were lysed in RLT lysis buffer containing β-mercaptoethanol (Qiagen, Valencia, Calif.).

Total RNA was isolated using the Rneasy kit (Qiagen, Valencia, Calif.) according to manufacturer's instructions and the RNA integrity was assessed by using an Agilent 2100 Bioanalyzer (Agilent, Palo Alto, Calif.). From 5 micrograms of total RNA, double-stranded cDNA containing the T7-dT (24) promoter sequence (Operon) was generated as a template for in vitro transcription single round amplification with biotin labels, using the Enzo R BioArray™ HighYield™ RNA Transcript Labeling Kit (Affymetrix Inc, Santa Clara, Calif.). Biotinylated cRNA targets were purified using the Sample Cleanup Module (Affymetrix), and subsequently hybridized to human U133A and B GeneChips (Affymetrix Inc, Santa Clara, Calif.) according to manufacturer's standard protocols. Arrays were scanned using a laser confocal scanner (Agilent).

Microarray Data Analysis. For each Affymetrix U133A and B GeneChip® raw intensity data were normalized to the mean intensity of all measurements on that array and scaled to a target intensity value of 500 (TGT) in Affymetrix Microarray Suite 5.0. Data were then further analyzed using GeneSpring software version 7.0. Data were notmalized to a set of healthy controls (sex and age matched). Affymetrix flag call of ‘present’ in at least 75% of samples of each cohort designated the filter of reliable intensity measurement from each individual gene chip. The combined two lists (17,231 probes) were used as quality control for statistical tests, class prediction and clustering algorithms subsequently performed on the data. Class comparison was performed using non-parametric ranking statistical analysis test (Mann-Whitney) applied to Quality Control genes. In the vertical direction, hierarchical clusters of genes were generated using the Pearson correlation around zero, Genespring's standard correlation measure. Class prediction was done using a supervised learning algorithm, K-Nearest Neighbors Method, which assigns a sample to pre-defined classes.

RT PCR. RNA samples were DNAse treated with TURBO DNA-free kit (Ambion, Austin, Tex.), total RNA for RT PCR analysis was further amplified due to low yields of total RNA. 5 μg of each RNA sample was converted to cDNA using the High Capacity cDNA Archive Kit (Applied Biosystems, Foster City, Calif.) in the Perkin Elmer GeneAmp PCR System 9600. Quantitative PCR was performed on selected targets using pre-developed primers and probe TaqMan ® Gene Expression Assays (Applied Biosystems, Foster City, Calif.) on the ABI Prism 7700 Sequence Detection System. Expression results were calculated as the difference in cycle threshold relative to the median of four healthy volunteers for each target confirmed.

Patient characteristics. The inventors sought to identify gene expression signatures discriminating SoJIA patients from healthy volunteers. PBMCs from 14 SoJIA patients displaying both systemic symptoms (fever and/or rash) and arthritis, 3 SoJIA patients with only systemic symptoms (fever, rash and/or pericarditis), and 16 healthy controls were analyzed. Of the 17 SoJIA patients, 14 were females and 3 males. In this group the patient demographics were as follows: 8 Hispanic, 7 Caucasian, 1 Asian and 1 African-American. Six patients were newly diagnosed and untreated at the time of blood draw. The remaining patients were receiving treatment with oral prednisone and/or IV Methyprednisolone pulses, Methotrexate and/or anti-TNF therapy (Table III). None of the patients had received IV pulses (Methylprednisolone or Infliximab) for at least 4 weeks prior to blood draw. The average time from initiation of symptoms to establishment of diagnosis and initiation of therapy in these patients was 6 months.

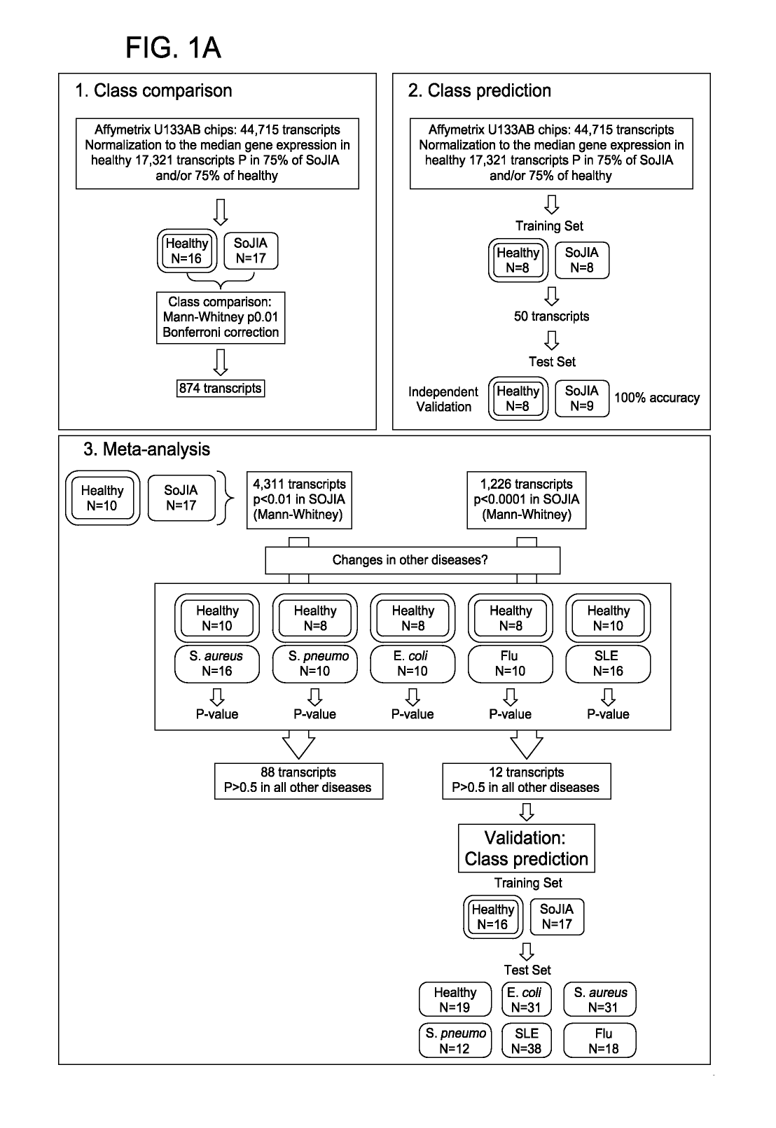

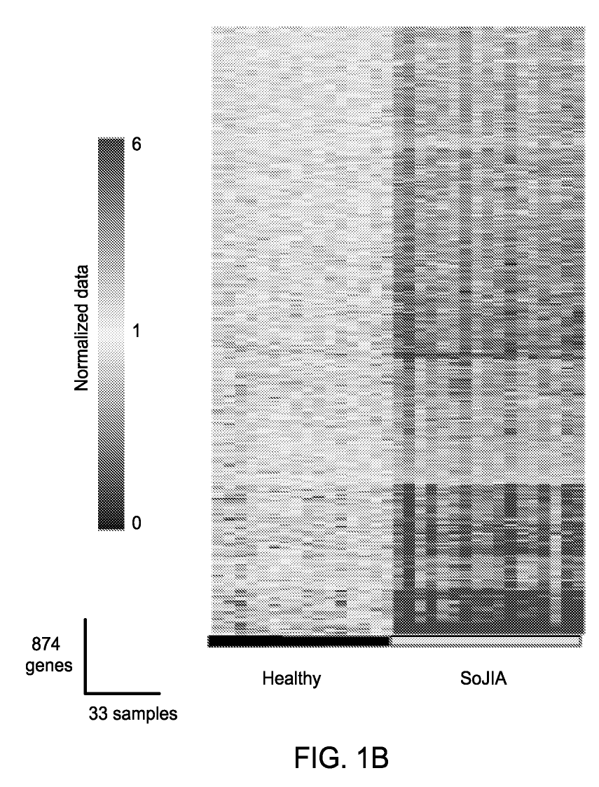

Blood leukocyte signatures differentiate SoJIA patients from healthy children. Statistical group comparisons were performed to identify genes whose expression would differentiate SoJIA patients from healthy controls, (non parametric Mann-Whitney rank test (p<0.001, and Bonferroni correction—the overall analysis strategy is presented in FIG. 1A). Transcripts displaying statistically significant differences (N=874) were ordered by hierarchical clustering (FIG. 1 and Table IV). The 50 most statistically significant genes are listed in Table IV (marked with an asterisk). Some of these genes encode proteins involved in ubiquitination (solute carrier family 6/SLC6A8), components of the erythrocyte cytoskeleton (EBP42, tropomodulin 1), and apoptosis (synuclein alpha). In the category of inflammatory/immune response related genes, the IL-1 receptor antagonist (IL-1RN) was one of the most significantly over-expressed transcripts. This is in agreement with the inventors' previous finding that IL-1 is an important mediator of this disease (9). The gene encoding the Fc Epsilon receptor was among the most significantly under-expressed. Transcription factors that play a role in immune/inflammatory responses were also found differentially expressed. GATA-3, for example, which drives T cells into the Th2 lineage (12), was under-expressed and Foxo3a, which has recently been shown to promote neutrophil survival in inflammatory arthritis (13), was found over-expressed.

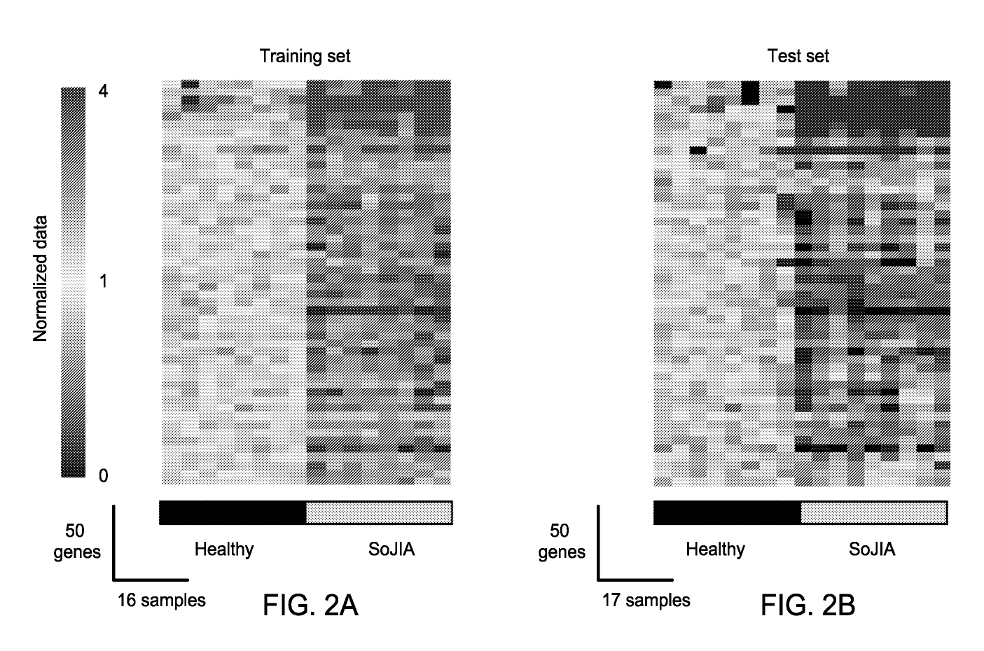

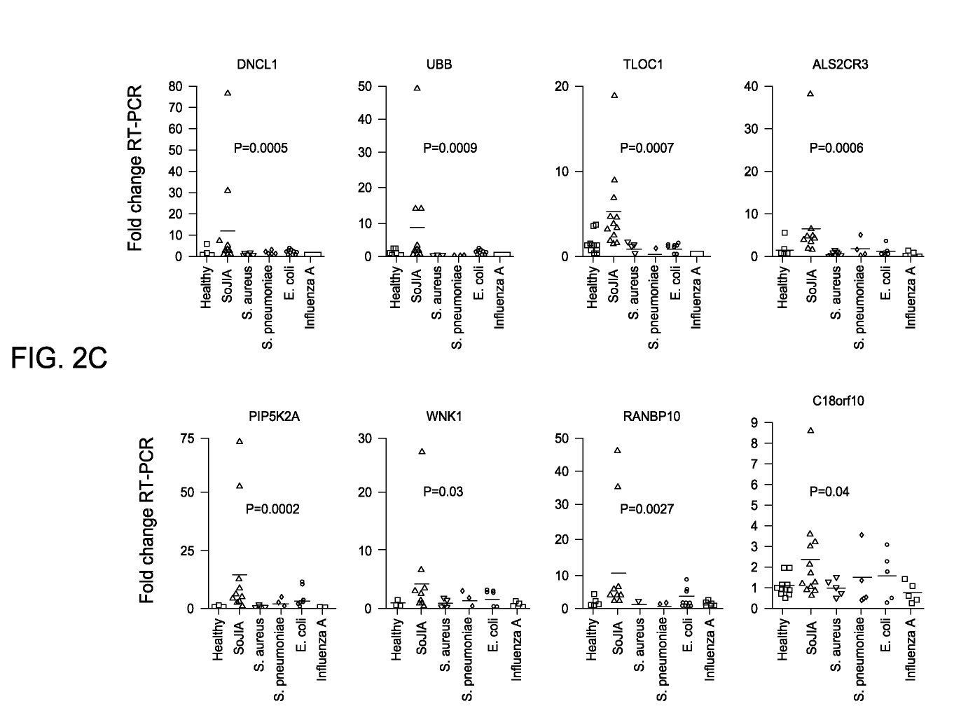

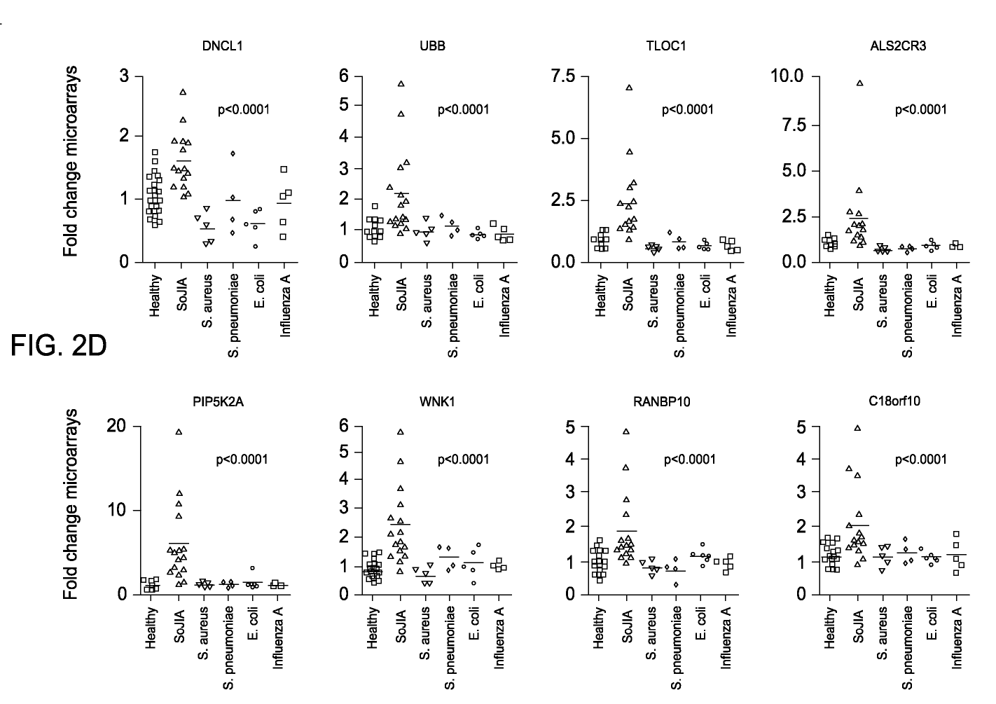

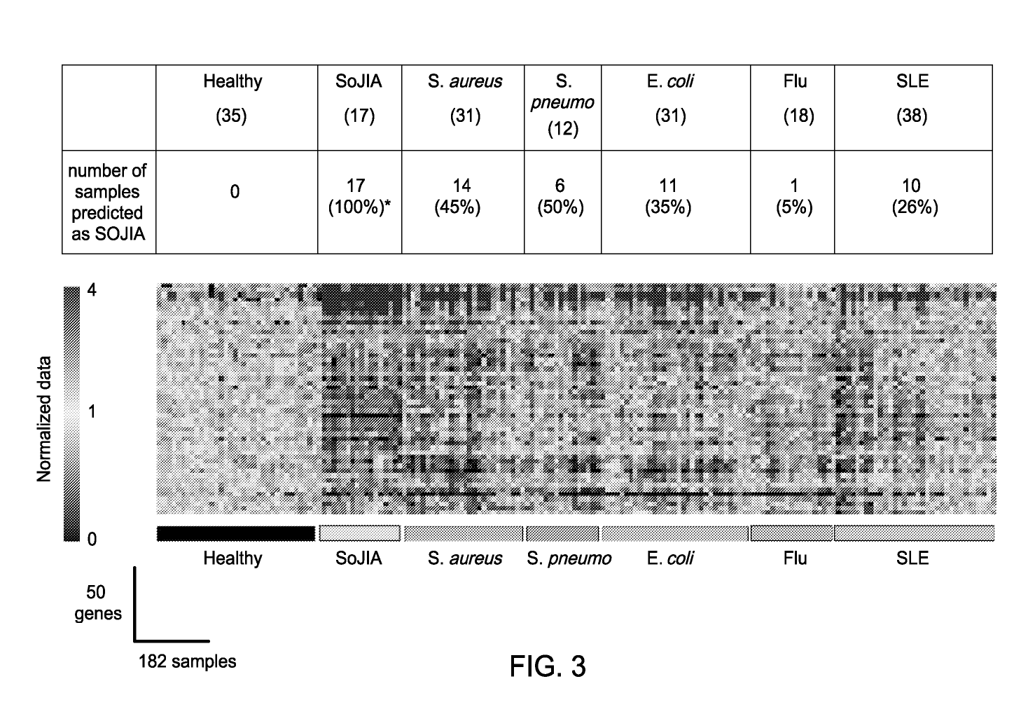

A diagnostic signature was identified by performing a two-step class prediction analysis: (1) Identification of classifier genes. The study groups included the initial class comparison analysis used to generate a 50 gene classifier capable of separating healthy volunteers from the SOJIA patient group based on differential gene expression. A subset of 8 healthy volunteers and 8 SoJIA patients were used in the training set (FIG. 2A). These transcripts were then evaluated within the same set of patients in a leave-one-out cross-validation scheme. Using this strategy, 100% of the healthy and 88% of the SOJIA samples were classified accurately (seven were predicted accurately and one was not predicted). (2) Independent validation of classifier genes. The ability of the above described sets of transcripts was studied to classify an independent test set composed of 8 healthy and 9 SOJIA. Using this approach, 100% of the patients were accurately classified (FIG. 2B).

Table I summarizes the list of transcripts that best discriminate SoJIA patients from healthy controls. Among these, genes encoding proteins involved in heme synthesis (delta hemoglobin and erythroid associated factor), erythrocyte-specific transcription factors (Kruppel-like factor 1), cytoskeleton (myosin light polypeptide 4) and the makorin 1 gene, which encodes a ubiquitin ligase modulating telomere length homeostasis (14).

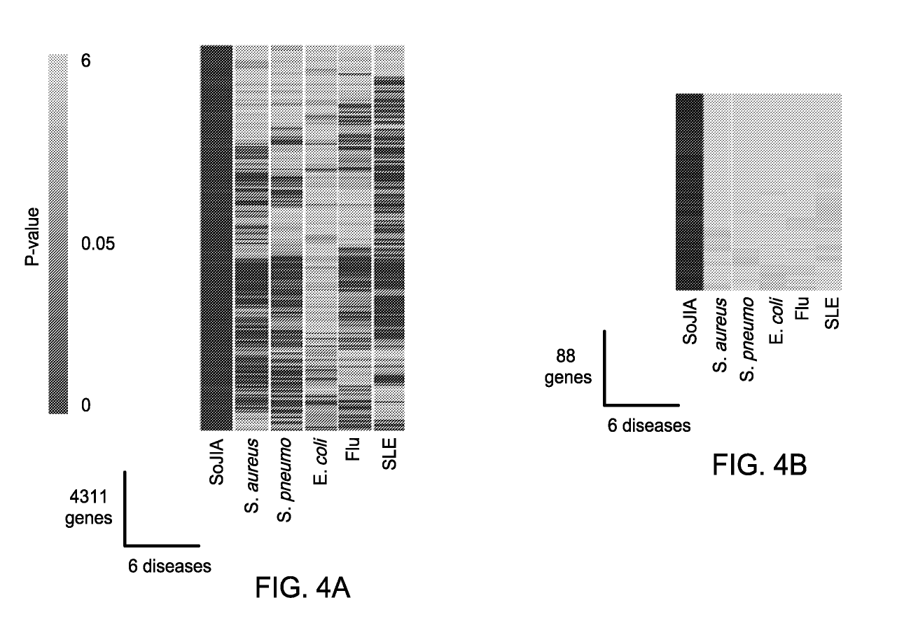

Specificity of the initial SoJIA signature. Children with SoJIA present with severe systemic symptoms (fever and rash) that usually precede the development of arthritis for weeks to years. Thus, the main differential diagnosis at presentation is an infectious disease. The ability of the 50 genes that discriminate SoJIA patients from healthy controls was tested to discriminate against a series of infections (31 patients with E. coli, 31 patients with S. aureus, 12 patients with S. pneumoniae and 18 patients with influenza A infections). As controls for non-infectious disease and steroid treatment, which included a group of 38 pediatric SLE patients. The 50 genes that discriminate SoJIA patients from healthy controls also identified patients with infections and SLE, as 45% of the S. aureus patients, 50% of the S. pneumoniae patients, 35% of the E. coli patients, 5% of the influenza A patients and 26 % of the SLE patients were incorrectly classified as SoJIA patients (FIG. 3). Thus, patients with SoJIA not only resemble patients with infections clinically, but they also display a remarkably similar signature. Next, a set of genes were identified that would uniquely characterize patients with SoJIA.

Identification of a specific SoJIA signature. In order to identify a diagnostic SoJIA signature the SoJIA group was compared to all the other patients. However, using this approach, a large proportion of the predictors genes differentially expressed in the infection/SLE groups versus SoJIA will in fact be expressed similarly in SoJIA patients and healthy controls. Furthermore, it is particularly difficult to control potentially confounding factors such as age or sex in comparisons that involve many groups of patients.

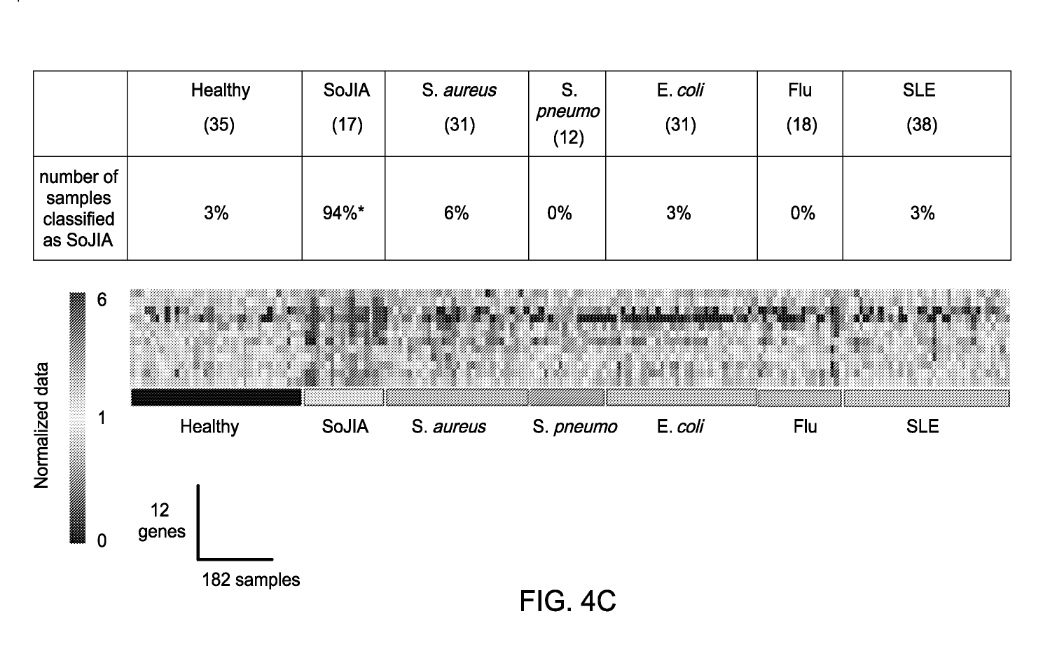

FIG. 1A summaries the new strategy for the identification of a SoJIA signature. First, a statistical comparison was performed between each group of patients (17 SoJIA, 10 influenza A, 10 E. coli, 10 S. pneumoniae, 16 S. aureus and 16 SLE) and their respective control groups composed of age-matched and gender-match healthy controls. The p-values obtained from each comparison were then subjected to selection criteria that permitted the identification of genes significantly changed in SoJIA patients, and not in any of the other groups. Overall, the “normalization” of each patient group to healthy control values and the comparison of significances rather than expression levels allows for more robust data comparisons. A non-stringent statistical group comparison (non parametric Mann-Whitney rank test, p<0.01) performed with 17 SoJIA and 10 healthy control samples yielded 4,311 differentially expressed transcripts (FIG. 4A). This analysis segregated transcripts that were the most specific to the study groups from those that were the more ubiquitous. The present inventors determined if the former would carry the signature of SoJIA. Thus, 88 transcripts were identified with an associated p-value<0.01 in SoJIA and >0.5 in all the other groups (FIG. 4B and Table III). None of these 88 best classifiers overlaps with the 50 genes that best discriminate SoJIA patients from healthy controls (Table I).

TABLE I
Fifty classifiers distinguishing SOJIA patients from healthy controls.

| | | | | |
|---|---|---|---|---|
| | | | Average | |
| | | | normalized | |
| | Gene | | values in | |
| Systematic | Symbol | p-value | SoJIA | Gene Title |

protein biosynthesis

| | | | | |
|---|---|---|---|---|
| 200002_at | RPL35 | 1.24E−08 | 0.6 | ribosomal protein L35 |
| 200089_s_at | RPL4 | 1.48E−10 | 0.6 | ribosomal protein L4 |
| 221726_at | RPL22 | 9.33E−10 | 0.7 | ribosomal protein L22 |
| 200802_at | SARS | 2.80E−09 | 0.8 | seryl-tRNA synthetase |
| 203113_s_at | EEF1D | 7.57E−11 | 0.2 | eukaryotic translation elongation factor |
| | | | | 1 delta (guanine nucleotide exchange |
| | | | | protein) |
| 212018_s_at | RSL1D1 | 2.82E−10 | 0.5 | ribosomal L1 domain containing 1 |

Ubiquitination

| | | | | |
|---|---|---|---|---|
| 209845_at | MKRN1 | 1.48E−10 | 4.2 | makorin, ring finger protein, 1 |
| 214790_at | SENP6 | 2.78E−09 | 0.5 | SUMO1/sentrin specific protease 6 |

Microtubule/Cytoskeleton

| | | | | |
|---|---|---|---|---|
| 210088_x_at | MYL4 | 7.57E−11 | 9.9 | myosin, light polypeptide 4, alkali; |
| | | | | atrial, embryonic |
| 212878_s_at | KNS2 | 7.57E−11 | 0.6 | kinesin 2 60/70 kDa |

Transcription

| | | | | |
|---|---|---|---|---|
| 209430_at | BTAF1 | 1.57E−07 | 0.6 | BTAF1 RNA polymerase II, B-TFIID |
| | | | | transcription factor-associated, 170 kDa |
| 210504_at | KLF1 | 7.57E−11 | 6.7 | Kruppel-like factor 1 (erythroid) |
| 217729_s_at | AES | 2.80E−09 | 0.5 | amino-terminal enhancer of split |
| 218490_s_at | ZNF302 | 4.68E−07 | 0.5 | zinc finger protein 302 |
| 203617_x_at | ELK1 | 6.59E−07 | 1.6 | ELK1, member of ETS oncogene |
| | | | | family |
| 226327_at | ZNF507 | 0.000972 | 0.7 | zinc finger protein 507 |
| 224518_s_at | ZNF559 | 4.70E−08 | 0.5 | zinc finger protein 559 |
| 204633_s_at | RPS6KA5 | 1.97E−08 | 0.6 | ribosomal protein S6 kinase, 90 kDa, |
| | | | | polypeptide 5 |
| 214177_s_at | PBXIP1 | 9.33E−10 | 0.6 | pre-B-cell leukemia transcription factor |
| | | | | interacting protein 1 |
| 200792_at | G22P1 | 4.69E−09 | 0.7 | thyroid autoantigen 70 kDa |
| | | | | (Ku antigen) |

Metabolism

| | | | | |
|---|---|---|---|---|
| 201050_at | PLD3 | 2.69E−05 | 1.4 | phospholipase D3 |
| 235802_at | C14orf175 | 7.57E−11 | 0.4 | chromosome 14 open reading frame |
| | | | | 175 |
| 226344_at | ZMAT1 | 4.27E−06 | 0.6 | zinc finger matrin type 1 |
| 212174_at | AK2 | 4.66E−09 | 0.7 | adenylate kinase 2 |

Immune response/Inflammatory response

| | | | | |
|---|---|---|---|---|
| 211734_s_at | FCER1A | 2.82E−10 | 0.3 | Fc fragment of IgE, high affinity I, |
| | | | | receptor for; alpha polypeptide |

Transport

| | | | | |
|---|---|---|---|---|
| 218978_s_at | MSCP | 2.82E−10 | 7.5 | mitochondrial solute carrier protein |
| 200063_s_at | NPM1 | 1.48E−10 | 0.5 | nucleophosmin (nucleolar |
| | | | | phosphoprotein B23, numatrin) |
| 210854_x_at | SLC6A8 | 7.57E−11 | 9.2 | solute carrier family 6 (neurotransmitter |
| | | | | transporter, creatine), member 8 |

Heme

| | | | | |
|---|---|---|---|---|
| 206834_at | HBD | 7.57E−11 | 36.4 | hemoglobin, delta |
| 219672_at | ERAF | 7.57E−11 | 30.0 | erythroid associated factor |

Apoptosis

| | | | | |
|---|---|---|---|---|
| 223518_at | DFFA | 2.82E−10 | 1.9 | DNA fragmentation factor, 45 kDa, |
| | | | | alpha polypeptide |

Unknown

| | | | | |
|---|---|---|---|---|
| 201537_s_at | DUSP3 | 0.000157 | 1.8 | dual specificity phosphatase 3 |
| | | | | (vaccinia virus phosphatase VH1- |
| | | | | related) |
| 203818_s_at | SF3A3 | 5.20E−10 | 0.7 | splicing factor 3a, subunit 3, 60 kDa |
| 209068_at | HNRPDL | 7.57E−11 | 0.5 | heterogeneous nuclear |
| | | | | ribonucleoprotein D-like |
| 212830_at | EGFL5 | 4.69E−09 | 1.9 | EGF-like-domain, multiple 5 |
| 213804_at | INPP5B | 1.06E−07 | 0.5 | inositol polyphosphate-5-phosphatase, |
| | | | | 75 kDa |
| 217807_s_at | GLTSCR2 | 7.47E−06 | 0.7 | glioma tumor suppressor candidate |
| | | | | region gene 2 |
| 218877_s_at | C6orf75 | 7.12E−08 | 0.5 | chromosome 6 open reading frame 75 |
| 220755_s_at | C6orf48 | 1.48E−10 | 0.6 | chromosome 6 open reading frame 48 |
| 221932_s_at | C14orf87 | 7.57E−11 | 10.7 | chromosome 14 open reading frame |
| | | | | 87 |
| 223011_s_at | OCIAD1 | 5.20E−10 | 0.6 | OCIA domain containing 1 |
| 223656_s_at | RP4-622L5 | 4.70E−08 | 1.5 | hypothetical protein RP4-622L5 |
| 225159_s_at | — | 1.06E−07 | 0.7 | — |
| 225180_at | TTC14 | 1.57E−07 | 0.6 | tetratricopeptide repeat domain 14 |
| 225845_at | BTBD15 | 1.97E−08 | 0.6 | BTB (POZ) domain containing 15 |
| 226544_x_at | MUTED | 2.11E−05 | 0.8 | muted homolog (mouse) |
| 226680_at | ZNFN1A5 | 3.06E−08 | 0.6 | zinc finger protein, subfamily 1A, 5 |
| 228122_at | LOC285331 | 0.000128 | 0.7 | hypothetical protein LOC285331 |
| 235587_at | LOC202781 | 7.57E−11 | 0.5 | hypothetical protein LOC202781 |
| 241863_x_at | — | 1.74E−06 | 0.5 | — |

Half of these genes (47/88) encode proteins with unknown function. Among those genes encoding proteins with known function, several are involved in microtubule/cytoskeleton reorganization, ubiquitination, cellular transport, apoptosis, metabolism, transcription, protein biosynthesis, and post-translational protein modification (Table II).

TABLE II
Best classifiers from meta-analysis of SoJIA vs infectious diseases and
SLE

| | | | | |
|---|---|---|---|---|
| | | | Average | |
| | | | normalized | |
| | Gene | | values in | |
| Probe Set ID | Symbol | p-value | SoJIA | Gene Title |

Microtubule/Cytoskeleton

| | | | | |
|---|---|---|---|---|
| 200703_at | DNCL1 | 2.16E−04 | 1.7 | dynein, cytoplasmic, light polypeptide 1 |
| 207490_at | TUBA4 | 3.96E−04 | 1.4 | tubulin, alpha 4 |

Extracellular matrix

| | | | | |
|---|---|---|---|---|
| 216993_s_at | COL11A2 | 0.00241 | 1.4 | collagen, type XI, alpha 2 |
| 202337_at | PMF1 | 9.06E−04 | 0.7 | polyamine-modulated factor 1 |

Ubiquitination

| | | | | |
|---|---|---|---|---|
| 200718_s_at | SKP1A | 0.00462 | 1.3 | S-phase kinase-associated protein 1A |
| | | | | (p19A) |
| 201824_at | RNF14 | 0.00301 | 2.0 | ring finger protein 14 |
| 210579_s_at | TRIM10 | 0.00835 | 1.4 | tripartite motif-containing 10 |

Transport

| | | | | |
|---|---|---|---|---|
| 201066_at | CYC1 | 5.27E−04 | 0.8 | cytochrome c-1 |
| 202125_s_at | ALS2CR3 | 5.27E−04 | 2.1 | amyotrophic lateral sclerosis 2 |
| | | | | (juvenile) chromosome region, |
| | | | | candidate 3 |
| 213415_at | CLIC2* | 1.69E−05 | 8.3 | chloride intracellular channel 2 |
| 215716_s_at | ATP2B1 | 0.00241 | 0.6 | ATPase, Ca++ transporting, plasma |
| | | | | membrane 1 |
| 218211_s_at | MLPH | 0.00462 | 1.5 | melanophilin |
| 224787_s_at | RAB18 | 6.94E−04 | 0.7 | RAB18, member RAS oncogene |
| | | | | family |
| 225352_at | TLOC1* | 1.10E−05 | 2.4 | translocation protein 1 |
| 226154_at | DNM1L | 0.00836 | 0.8 | Dynamin 1-like |
| 238066_at | RBP7 | 0.00836 | 0.8 | retinal binding protein 7, cellular |
| 244227_at | SYT6 | 0.00241 | 1.3 | synaptotagmin VI |

Apoptosis

| | | | | |
|---|---|---|---|---|
| 212373_at | FEM1B | 5.27E−04 | 0.7 | Fem-1 homolog b (C. elegans) |
| 235116_at | TRAF1 | 9.06E−04 | 1.3 | TNF receptor-associated factor 1 |

Metabolism

| | | | | |
|---|---|---|---|---|
| 209301_at | CA2 | 0.00374 | 2.6 | carbonic anhydrase II |
| 209509_s_at | DPAGT1 | 0.0015 | 1.2 | dolichyl-phosphate N- |
| | | | | acetylglucosaminephosphotransferase 1 |

Transciption

| | | | | |
|---|---|---|---|---|
| 202484_s_at | MBD2 | 0.00191 | 0.7 | methyl-CpG binding domain protein 2 |
| 224099_at | KCNH7 | 0.00191 | 1.5 | potassium voltage-gated channel, |
| | | | | subfamily H (eag-related), member 7 |
| 224933_s_at | JMJD1C | 0.00374 | 0.7 | jumonji domain containing 1C |
| 225527_at | CEBPG | 0.00117 | 0.7 | CCAAT/enhancer binding protein |
| | | | | (C/EBP), gamma |
| 227685_at | TMF1 | 0.0069 | 0.8 | TATA element modulatory factor 1 |
| 228785_at | ZNF281 | 0.00241 | 0.6 | Zinc finger protein 281 |
| 235389_at | PHF20 | 0.00462 | 0.8 | PHD finger protein 20 |
| 35671_at | GTF3C1 | 2.16E−04 | 1.3 | general transcription factor IIIC, |
| | | | | polypeptide 1, alpha 220 kDa |

Nuclear mRNA splicing, via spliceosome

| | | | | |
|---|---|---|---|---|
| 223416_at | SF3B14 | 0.00241 | 0.8 | splicing factor 3B, 14 kDa subunit |
| 225394_s_at | MADP-1* | 2.62E−06 | 0.6 | MADP-1 protein |

Glysocylation

| | | | | |
|---|---|---|---|---|
| 201724_s_at | GALNT1 | 0.00462 | 0.9 | UDP-N-acetyl-alpha-D- |
| | | | | galactosamine:polypeptide N- |
| | | | | acetylgalactosaminyltransferase 1 |
| 210205_at | B3GALT4 | 5.27E−04 | 1.3 | UDP-Gal:betaGlcNAc beta 1,3- |
| | | | | galactosyltransferase, polypeptide 4 |

Phosphorylation

| | | | | |
|---|---|---|---|---|
| 211992_at | WNK1 | 5.27E−04 | 2.1 | WNK lysine deficient protein kinase 1 |
| 226979_at | MAP3K2 | 0.00567 | 0.7 | Mitogen-activated protein kinase |
| | | | | kinase kinase 2 |
| 227073_at | MAP3K2 | 0.00836 | 0.8 | Mitogen-activated protein kinase |
| | | | | kinase kinase 2 |

Protein Biosynthesis

| | | | | |
|---|---|---|---|---|
| 212225_at | SUI1 | 2.16E−04 | 0.6 | Putative translation initiation factor |
| 224302_s_at | MRPS36 | 0.00374 | 0.8 | mitochondrial ribosomal protein S36 |
| 226296_s_at | MRPS15* | 3.80E−05 | 0.6 | mitochondrial ribosomal protein S15 |

Protein folding

| | | | | |
|---|---|---|---|---|
| 201759_at | TBCD | 1.12E−04 | 2.2 | tubulin-specific chaperone d |
| 225061_at | DNAJA4 | 0.00191 | 2.4 | DnaJ (Hsp40) homolog, subfamily A, |
| | | | | member 4 |
| 228622_s_at | DNAJC4* | 3.80E−05 | 0.7 | DnaJ (Hsp40) homolog, subfamily C, |
| | | | | member 4 |

Unknown

| | | | | |
|---|---|---|---|---|
| 211994_at | —* | 2.62E−06 | 2.8 | Clone A9A2BRB5 (CAC)n/(GTG)n |
| | | | | repeat-containing mRNA |
| 212055_at | C18orf10* | 5.54E−05 | 2.0 | chromosome 18 open reading frame |
| | | | | 10 |
| 212174_at | AK2* | 8.80E−07 | 0.7 | adenylate kinase 2 |
| 212341_at | MGC21416 | 0.00836 | 1.6 | hypothetical protein MGC21416 |
| 212829_at | — | 6.94E−04 | 2.0 | CDNA FLJ13267 fis, clone |
| | | | | OVARC1000964 |
| 216739_at | — | 3.96E−04 | 1.6 | — |
| 218116_at | C9orf78 | 0.00191 | 2.1 | chromosome 9 open reading frame 78 |
| 218126_at | FLJ10579 | 9.06E−04 | 1.5 | hypothetical protein FLJ10579 |
| 218583_s_at | RP42 | 0.00462 | 1.5 | RP42 homolog |
| 218936_s_at | HSPC128 | 0.00117 | 0.6 | HSPC128 protein |
| 222309_at | C6orf62 | 0.00567 | 0.6 | Chromosome 6 open reading frame |
| | | | | 62 |
| 223112_s_at | NDUFB10 | 3.96E−04 | 0.8 | NADH dehydrogenase (ubiquinone) 1 |
| | | | | beta subcomplex, 10, 22 kDa |
| 223548_at | C1orf26 | 0.0015 | 1.4 | chromosome 1 open reading frame 26 |
| 224807_at | KIAA1533 | 0.0015 | 0.8 | KIAA1533 |
| 224915_x_at | — | 9.06E−04 | 0.7 | Similar to RPE-spondin |
| 225202_at | RHOBTB3 | 0.0069 | 1.2 | Rho-related BTB domain containing 3 |
| 225213_at | TA-PP2C | 2.16E−04 | 0.8 | T-cell activation protein phosphatase |
| | | | | 2C |
| 225819_at | TBRG1 | 0.00241 | 0.7 | transforming growth factor beta |
| | | | | regulator 1 |
| 226833_at | FLJ32499 | 0.00301 | 1.3 | hypothetical protein FLJ32499 |
| 226927_at | — | 0.00374 | 1.2 | Homo sapiens, clone |
| | | | | IMAGE: 3894337, mRNA |
| 227265_at | — | 0.00301 | 0.8 | MRNA; cDNA DKFZp686N07104 |
| 228452_at | C17orf39 | 0.00625 | 1.6 | chromosome 17 open reading frame |
| | | | | 39 |
| 228953_at | KIAA1971* | 5.54E−05 | 0.6 | similar to junction-mediating and |
| | | | | regulatory protein p300 JMY |
| 229074_at | EHD4 | 0.00117 | 0.8 | EH-domain containing 4 |
| 229653_at | FLJ10979 | 0.00836 | 1.4 | Hypothetical protein FLJ10979 |
| 230118_at | — | 2.16E−04 | 1.3 | Transcribed locus |
| 230421_at | LOC345462 | 0.00567 | 1.2 | similar to hypothetical protein |
| | | | | 9630041N07 |
| 230546_at | KIAA1036* | 7.95E−05 | 1.6 | KIAA1036 |
| 230747_s_at | —* | 3.80E−05 | 0.7 | CDNA clone IMAGE: 3029742, partial |
| | | | | cds |
| 232486_at | LRFN1 | 0.00462 | 1.4 | leucine rich repeat and fibronectin |
| | | | | type III domain containing 1 |
| 232709_at | — | 0.00191 | 0.7 | CDNA FLJ13427 fis, clone |
| | | | | PLACE1002477 |
| 233469_at | psiTPTE22 | 0.00301 | 1.3 | TPTE pseudogene |
| 234305_s_at | MLZE | 9.06E−04 | 1.4 | melanoma-derived leucine zipper, |
| | | | | extra-nuclear factor |
| 235798_at | — | 0.00117 | 0.8 | — |
| 236196_at | — | 0.0015 | 0.7 | CDNA FLJ42548 fis, clone |
| | | | | BRACE3004996 |
| 241491_at | KIAA1002 | 6.94E−04 | 1.5 | KIAA1002 protein |
| 241517_at | — | 0.00117 | 1.3 | — |
| 241817_at | FLJ43654 | 3.96E−04 | 0.7 | FLJ43654 protein |
| 242003_at | LOC157697 | 0.00301 | 0.7 | Hypothetical protein LOC157697 |
| 242300_at | —* | 2.56E−05 | 4.0 | — |
| 243109_at | MCTP2 | 2.94E−04 | 1.7 | Multiple C2-domains with two |
| | | | | transmembrane regions 2 |
| 243434_at | FLJ10874 | 0.00836 | 1.2 | Hypothetical protein FLJ10874 |
| 244092_at | ZRANB3 | 0.0015 | 1.4 | Zinc finger, RAN-binding domain |
| | | | | containing 3 |
| 244390_at | — | 0.0015 | 1.8 | Transcribed locus |
| 244728_at | LOC130063 | 0.00462 | 1.4 | hypothetical gene LOC130063 |
| 53987_at | RANBP10 | 2.94E−04 | 1.8 | RAN binding protein 10 |

By increasing the stringency of the analysis, 12 genes were identified displaying a p-value<0.0001 in SOJIA and therefore showing the highest degree of specificity for this condition in comparison with the 5 disease groups used as reference (p-value<0.001 in SoJIA and >0.5 in all the other groups). These 12 genes are included (marked with an asterisk) in Table II. Overall, 7/12 encode proteins with unknown function.

TABLE III
Patients' clinical data.
SOJIA

| | | | | | | | | Healthy |
|---|---|---|---|---|---|---|---|---|
| | | | | | Active | | meta- | vs SOJIA |
| sample | age | Ethnicity | sex | Fever/Rash | arthritis | medication | analysis | prediction |
| sys15 | 2 | Hispanic | F | yes | yes | Steroids | yes | Test |
| | | | | | | PO, | | |
| | | | | | | Steroids IV, | | |
| | | | | | | Methotrexate, | | |
| | | | | | | Infliximab | | |
| sys51 | 2 | White | F | yes | yes | Steroids | yes | Test |
| | | | | | | PO, | | |
| | | | | | | Steroids IV | | |
| sys12 | 4 | White | F | yes | yes | Steroids | yes | Test |
| | | | | | | PO, | | |
| | | | | | | Methotrexate | | |
| sys56 | 4 | Hispanic | F | yes | yes | Steroids | yes | Test |
| | | | | | | PO, | | |
| | | | | | | Steroids IV, | | |
| | | | | | | Methotrexate | | |
| sys21 | 4 | Asian | F | yes | yes | Steroids | yes | Test |
| | | | | | | PO, | | |
| | | | | | | Methotrexate | | |
| sys39 | 5 | Hispanic | F | yes | yes | Steroids | yes | Training |
| | | | | | | PO, | | |
| | | | | | | Steroids IV | | |
| sys12 | 6 | White | F | yes | yes | Steroids | yes | Training |
| | | | | | | PO, Methotrexate, | | |
| | | | | | | Infliximab | | |
| sys68 | 6 | Hispanic | F | yes | yes | None | yes | Training |
| sys32 | 9 | White | F | yes | yes | Steroids | yes | Test |
| | | | | | | PO | | |
| sys53 | 9 | White | F | yes | yes | None | yes | Test |
| sys62 | 9 | Hispanic | F | yes | yes | Steroids | yes | Test |
| | | | | | | PO, | | |
| | | | | | | Steroids IV | | |
| sys02 | 11 | White | F | yes | yes | Steroids | yes | Training |
| | | | | | | PO, | | |
| | | | | | | Methotrexate, | | |
| | | | | | | Infliximab | | |
| sys52 | 14 | White | F | yes | yes | None | yes | Test |
| sys33 | 17 | Hispanic | F | yes | yes | Steroids | yes | Training |
| | | | | | | PO, | | |
| | | | | | | Steroids IV, | | |
| | | | | | | Methotrexate | | |
| sys78 | 3 | White | M | yes | no | None | yes | Training |
| sys16 | 4 | Hispanic | M | yes | yes | None | yes | Training |
| sys81 | 6 | Black | M | yes | yes | None | yes | Training |

| | | | | | | |
|---|---|---|---|---|---|---|
| | | | | | | meta- |
| sample | age | Ethnicity | sex | clinical disease | medication | analysis |

S. aureus

| | | | | | | |
|---|---|---|---|---|---|---|
| 5 | 10 | Hispanic | M | Osteomyelitis | Cefazolin | yes |
| 24 | 3 | Black | M | Osteomyelitis | Vancomycin, | yes |
| | | | | | Rifampin | |
| 30 | 15 | Black | M | Bacteremia | Vancomycin | no |
| 40 | 12 | White | M | Osteomyelitis, | Cefazolin | yes |
| | | | | Bacteremia | | |
| 43 | 7 | Black | M | Hip abscess, | Vancomycin, | yes |
| | | | | Bacteremia | Rifampin | |
| 62 | 2 | White | M | Osteomyelitis | Clindamycin | no |
| 66 | 3 m | Black | F | Pneumonia | Vancomycin, | no |
| | | | | | Gentamicin | |
| 67 | 7 | White | F | Osteomyelitis, | Vancomycin, | yes |
| | | | | Bacteremia | Rifampin | |
| 69 | 9 m | Hispanic | M | Lung abscess | Vancomycin, | no |
| | | | | | Cefazolin | |
| 70 | 15 m | White | F | Abscess | Vancomycin | no |
| 84 | 18 | Black | F | Abscess | Cefazolin | yes |
| 88 | 11 m | Hispanic | M | Osteomyelitis, | Vancomycin | no |
| | | | | bacteremia | | |
| 89 | 4 m | Black | F | Abscess | Clindamycin | no |
| 90 | 8 m | Black | M | Septicarthritis | Oxacillin | no |
| 150 | 9 | Black | F | Osteomyelitis, | Vancomycin, | yes |
| | | | | Bacteremia | Rifampin | |
| 179 | 12 | White | M | Endocarditis, | Oxacillin, Gentamicin, | no |
| | | | | Bacteremia. | Rifampin | |
| 205 | 7 | Hispanic | M | Pneumonia, | Vancomycin | yes |
| | | | | Bacteremia | | |
| 206 | 1 | Hispanic | F | Abscess | Clindamycin | no |
| 208 | 10 | White | F | Osteomyelitis, | Clindamycin, | yes |
| | | | | Bacteremia, pneumonia | Rifampin, Vancomycin | |
| 216 | 10 | Hispanic | F | Osteomyelitis, | Vancomycin, | yes |
| | | | | Bacteremia | Rifampin | |
| 220 | 11 | Hispanic | M | Osteomyelitis, | Cefazolin, Rifampin | yes |
| | | | | Bacteremia | | |
| 221 | 6 | Black | F | Osteomyelitis, | Vancomycin, | yes |
| | | | | Bacteremia | Rifampin | |
| 224 | 10 | White | M | Osteomyelitis, | Oxacillin, Rifampin | yes |
| | | | | Bacteremia | | |
| 241 | 10 m | Black | F | Pneumonia, Bacteremia | Vancomycin, | no |
| | | | | | Rifampin | |
| 242 | 13 m | Black | M | Abscess, Bacteremia | Clindamycin | no |
| 258 | 8 | White | F | Osteomyelitis, | Cefazolin | yes |
| | | | | Bacteremia | | |
| 262 | 13 | Hispanic | M | Abscess, | Clindamycin | no |
| | | | | Bacteremia | | |
| 264 | 13 | Black | M | Septic arthritis | gentamicin, | no |
| | | | | | Vancomycin, | |
| | | | | | cefazolin, | |
| 271 | 13 | Black | M | Osteomyelitis | Clindamycin | no |
| 281 | 3 | White | F | Osteomyelitis | Clindamycin | yes |
| 315 | 3 | Hispanic | F | Cellulitis | Vancomycin | yes |

S. pn.

| | | | | | | |
|---|---|---|---|---|---|---|
| 9 | 4 m | White | M | Abscess | Cefazolin | yes |
| 25 | 2 m | Hispanic | M | Meningitis | Ampicillin, Ceftriaxone | no |
| 41 | 23 m | White | F | Pneumonia, Empyema | Ceftriaxone | yes |
| 96 | 16 m | Hispanic | M | Pneumonia, Empyema | Ceftriaxone, | yes |
| | | | | | Azithromycin | |
| 113 | 7 m | Hispanic | F | Septic arthritis | Ceftriaxone, | yes |
| | | | | | clindamycin | |
| 155 | 3 m | Hispanic | M | Meningitis | Ceftriaxone, | no |
| | | | | | Vancomycin | |
| 261 | 13 | White | M | Meningitis | Ceftriaxone, | yes |
| | | | | | vancomycin | |
| 265 | 2 | White | F | Empyema | Ceftriaxone, | yes |
| | | | | | vancomycin | |
| 268 | 3 | Hispanic | M | Empyema | Ceftriaxone, | yes |
| | | | | | clindamycin | |
| 277 | 16 | White | M | Empyema | Ceftriaxone, | yes |
| | | | | | vancomycin | |
| 287 | 3 | White | F | Pneumonia, bacteremia | Ceftriaxone, | yes |
| | | | | | vancomycin | |
| 289 | 2 | Hispanic | M | Empyema | Ceftriaxone, | yes |
| | | | | | vancomycin | |

Flu

| | | | | | | |
|---|---|---|---|---|---|---|
| 55 | 11 m | Hispanic | M | Respiratory | Cefuroxime | yes |
| | | | | distress | | |
| 87 | 19 m | White | F | Fever, Hypoxia | Cefuroxime | yes |
| 92 | 1 m | Hispanic | F | Fever | Ampicillin, Ceftriaxone | no |
| 95 | 4 | Hispanic | M | Fever | None | yes |
| 101 | 4 m | Hispanic | M | Fever, URI | Cefuroxime, | no |
| | | | | | Oseltamivir | |
| 104 | 17 m | Hispanic | M | Seizures, Fever, | Ceftriaxone | yes |
| | | | | Respiratory | | |
| | | | | failure | | |
| 105 | 4 | Hispanic | F | Fever, Encephalopathy | Ceftriaxone, | yes |
| | | | | | Aciclovir, Oseltamivir | |
| 107 | 1.5 m | Asian | M | Fever, Lethargy | Ampicillin, Ceftriaxone | no |
| 108 | 5 m | Hispanic | M | Fever | Ceftriaxone | no |
| 112 | 1 m | Hispanic | M | Fever, URI | Ampicillin, Gentamycin | no |
| 114 | 18 m | Black | F | Respiratory | Cefuroxime, | yes |
| | | | | distress, fever | Oseltamivir | |
| 115 | 20 m | White | M | Seizures | Amoxicillin | yes |
| 116 | 2 | White | M | Fever, URI | Cefuroxime, | no |
| | | | | | Clindamycin | |
| 117 | 24 | White | F | Fever | None | yes |
| 128 | 11 m | Hispanic | F | Fever, Hypoxia | Cefuroxime | yes |
| 132 | 6 m | White | M | Respiratory | Oxacillin, Tobramycin | yes |
| | | | | distress, fever | | |
| 259 | 3 m | Hispanic | F | Pneumonia | None | no |
| 266 | 36 | White | F | Fever, cough | None | no |

E. coli

| | | | | | | |
|---|---|---|---|---|---|---|
| 12 | 5 m | Black | M | Bacteremia | Ceftriaxone | yes |
| 13 | 5 m | White | F | UTI | Ceftriaxone | yes |
| 31 | 3 m | Hispanic | F | UTI, bacteremia | Gentamicin | no |
| 34 | 16 | White | F | Pyelonephritis | Gentamicin | yes |
| 48 | 2 m | White | M | UTI | Ampicillin, ceftriaxone | no |
| 57 | 3 m | Black | F | UTI, bacteremia | Gentamicin | no |
| 74 | 4 m | Hispanic | F | UTI, bacteremia | Ceftriaxone | no |
| 82 | 2 m | Hispanic | M | UTI | Ampicillin, ceftriaxone | no |
| 86 | 3 m | Hispanic | M | UTI | Ceftriaxone | yes |
| 118 | 1.5 m | White | M | UTI | | no |
| 120 | 1.5 m | Hispanic | M | UTI | Ampicillin, ceftriaxone | no |
| 133 | 2 m | Hispanic | M | UTI | Ceftriaxone | yes |
| 139 | 1 m | Hispanic | M | UTI | Ampicillin, ceftriaxone | no |
| 148 | 8 | Hispanic | F | UTI | Ceftriaxone | yes |
| 151 | 1.5 m | Hispanic | M | UTI | Ampicillin, gentamicin | no |
| 152 | 2.5 m | Black | M | Bacteremia, meningitis | Ceftriaxone, gentamicin | no |
| 154 | 2 m | Hispanic | M | UTI | Ceftriaxone | Yes |
| 156 | 3 m | Hispanic | M | UTI | | Yes |
| 161 | 1.7 m | Hispanic | M | UTI | Ampicillin, ceftriaxone | no |
| 168 | 3 m | White | F | UTI | Ceftriaxone | no |
| 171 | 3 m | Hispanic | F | UTI | Ceftriaxone | no |
| 175 | 0.5 m | Hispanic | F | UTI, bacteremia | Ceftriaxone | no |
| 180 | 1 m | Hispanic | M | UTI | Ampicillin, gentamicin | no |
| 183 | 1.5 m | Hispanic | M | UTI | Ampicillin, gentamicin, | no |
| | | | | | ceftriaxone | |
| 184 | 0.5 m | White | F | UTI, bacteremia | Ampicillin, gentamicin | no |
| 188 | 1.5 m | White | M | UTI | Ampicillin, gentamicin, | no |
| | | | | | ceftriaxone | |
| 197 | 1.25 m | White | M | UTI | Ampicillin, gentamicin | no |
| 219 | 5 m | White | F | UTI, bacteremia | Ceftriaxone | yes |
| 222 | 3 m | Hispanic | F | UTI, bacteremia | Ceftriaxone, | no |
| | | | | | gentamicin | |
| 229 | 4 m | Hispanic | F | UTI, bacteremia | Ceftriaxone | yes |

SLE

| | | | | | | meta- |
|---|---|---|---|---|---|---|
| sample | age | Ethnicity | sex | SLEDAI | medication | analysis |
| SLE36 | 16 | Hispanic | F | 20 | None | yes |
| SLE61 | 16 | Black | M | 10 | Steroids | no |
| | | | | | PO, | |
| | | | | | Cellcept | |
| SLE149 | 15 | Other | M | 20 | None | no |
| SLE56 | 17 | Black | F | 20 | Steroids | no |
| | | | | | PO and IV, | |
| | | | | | Cytoxan | |
| SLE83 | 8 | Black | F | 2 | Steroids | no |
| | | | | | PO | |
| SLE35 | 16 | Hispanic | M | 8 | Steroids | no |
| | | | | | PO and IV | |
| | | | | | and | |
| | | | | | Cellcept | |
| SLE88 | 13 | White | F | 18 | Steroids | yes |
| | | | | | PO and IV, | |
| | | | | | Cytoxan | |
| SLE27 | 13 | Black | F | 14 | Steroids | no |
| | | | | | PO and | |
| | | | | | Cytoxan | |
| SLE100 | 13 | Hispanic | F | 4 | Steroids | yes |
| | | | | | PO and IV | |
| SLE105 | 12 | Hispanic | F | 16 | Steroids | yes |
| | | | | | PO, | |
| | | | | | Cellcept | |
| SLE106 | 14 | Black | F | 4 | Steroids | no |
| | | | | | PO | |
| SLE108 | 17 | White | F | 10 | None | no |
| SLE111 | 15 | Hispanic | F | 18 | None | no |
| SLE113 | 13 | Asian | F | 24 | None | yes |
| SLE114 | 14 | Black | F | 14 | None | yes |
| SLE121 | 12 | Hispanic | F | 10 | Steroids | yes |
| | | | | | PO | |
| SLE20 | 12 | Black | F | 16 | Steroids | no |
| | | | | | PO, | |
| | | | | | Cellcept | |
| SLE31 | 8 | Hispanic | F | 8 | None | yes |
| SLE43 | 15 | Hispanic | F | 12 | Steroids | no |
| | | | | | PO, | |
| | | | | | Cytoxan | |
| SLE45 | 9 | Black | F | 16 | Steroids | yes |
| | | | | | PO, | |
| | | | | | Cytoxan | |
| SLE50 | 15 | Black | F | 2 | None | no |
| SLE51 | 13 | Hispanic | F | 11 | None | yes |
| SLE53 | 17 | Hispanic | F | 2 | Steroids | no |
| | | | | | PO | |
| SLE57 | 12 | Black | M | 10 | Steroids | yes |
| | | | | | PO, | |
| | | | | | Steroids IV | |
| SLE60 | 12 | Hispanic | F | 4 | Steroids | yes |
| | | | | | PO, | |
| | | | | | Steroids IV | |
| SLE62 | 14 | Hispanic | F | 2 | Steroids | no |
| | | | | | PO, | |
| | | | | | Steroids IV | |
| SLE67 | 16 | White | F | 2 | Imuran | no |
| SLE74 | 17 | Black | F | 6 | Steroids | no |
| | | | | | PO | |
| SLE75 | 17 | Hispanic | F | 6 | None | no |
| SLE79 | 10 | Hispanic | F | 8 | None | yes |
| SLE82 | 17 | Black | F | 8 | Steroids | no |
| | | | | | PO | |
| SLE85 | 16 | Black | F | 12 | None | no |
| SLE90 | 11 | Hispanic | F | 4 | Steroids | yes |
| | | | | | PO, | |
| | | | | | Steroids IV | |
| SLE91 | 10 | Asian | F | 12 | Steroids | yes |
| | | | | | PO | |
| SLE96 | 16 | Hispanic | F | 4 | Steroids | no |
| | | | | | PO | |
| SLE65 | 10 | Hispanic | F | 8 | Steroids | no |
| | | | | | PO, | |
| | | | | | Steroids IV, | |
| | | | | | Cytoxan | |
| SLE21 | 10 | Hispanic | F | 12 | None | no |
| SLE17 | 14 | Asian | M | 2 | Steroids | yes |
| | | | | | PO | |

Healthy

| | | | | | | | | | | Healthy |
|---|---|---|---|---|---|---|---|---|---|---|
| | | | | Meta-analysis | | | | | | vs |
| | | | | SOJIA | E coli | Flu | Staph | Strep | SLE | SOJIA |
| sample | age | Ethnicity | sex | vs H | vs H | vs H | vs H | vs H | vs H | prediction |
| H46 | 5 | White | F | yes | no | no | no | no | yes | Training |
| H27 | 6 | White | F | yes | no | no | no | no | yes | Training |
| H36 | 7 | White | F | yes | no | no | no | no | yes | Training |
| H37 | 8 | White | F | yes | no | no | no | no | yes | Training |
| H148 | 8 | White | F | yes | no | no | no | no | yes | Training |
| JC | 8 | Hispanic | M | yes | no | no | no | no | yes | Training |
| H42 | 9 | Hispanic | M | yes | no | no | no | no | yes | Test |
| H30 | 10 | Hispanic | F | yes | no | no | no | no | yes | Test |
| SC | 10 | White | F | no | no | no | no | no | no | Test |
| H28 | 11 | Hispanic | F | no | no | no | no | no | no | Test |
| H45 | 12 | AA | F | no | no | no | no | no | no | Test |
| H149 | 12 | Hispanic | M | yes | no | no | no | no | yes | Test |
| JM | 13 | Hispanic | F | no | no | no | no | no | no | Test |
| H150 | 13 | White | M | no | no | no | no | no | no | Training |
| BW | 14 | White | M | yes | no | no | no | no | yes | Training |
| H98 | 16 | White | F | no | no | no | no | no | no | Test |
| 45 | 4 | O | F | no | no | yes | no | yes | no | |
| 11N | 4 | A | F | no | no | yes | yes | no | no | |
| 12N | 1.83 | C | M | no | yes | yes | no | yes | no | |
| 19N | 0.25 | Hispanic | M | no | yes | yes | no | yes | no | |
| 20N | 0.83 | Hispanic | M | no | yes | yes | no | no | no | |
| 23N | 7 | Hispanic | F | no | yes | no | yes | no | no | |
| 24N | 3 | C | M | no | no | no | yes | no | no | |
| 25N | 0.91 | Hispanic | F | no | yes | yes | no | no | no | |
| 26N | 7 | A | F | no | no | no | yes | no | no | |
| 27N | 0.83 | C | M | no | yes | no | no | yes | no | |
| 3N | 6 | Hispanic | M | no | no | no | yes | no | no | |
| 7N | 1.58 | A | F | no | yes | yes | no | yes | no | |
| 8N | 10 | A | M | no | no | no | yes | no | no | |
| 204 | 2 | C | M | no | no | no | no | yes | no | |
| 294 | 12 | B | F | no | no | no | yes | yes | no | |
| 295 | 9.5 | B | F | no | no | no | no | no | no | |
| 300 | 17 | White | M | no | yes | yes | yes | yes | no | |
| 301 | 8.2 | White | M | no | no | no | yes | no | no | |
| 303 | 6.3 | White | F | no | no | no | yes | no | no | |

Twelve genes identified through mieta-analysis can be used to diagnose SoJIA. The ability of this set of 12 genes to specifically identify patients with SoJIA was then evaluated. A training set composed of 16 healthy and 17 SoJIA samples was used to predict sample class for a cohort of 35 healthy, 31 E. coli, 31 S. aureus, 12 S. pneumoniae, 18 influenza A and 38 SLE patients. While this analysis allowed us to classify SoJIA patients with 94% accuracy, very few samples from the other groups displayed a gene expression signature that was sufficiently close to that of SoJIA patients to be classified as such: 6% of S. aureus, 3% of E. coli, 3% of SLE and none of the S. pneumoniae and influenza A samples. The gene tree corresponding to these transcripts in individual patients and controls is displayed in FIG. 4C.

In order to validate the microarray data, RT-PCR was performed on 8 of these 12 genes. RNA samples were obtained from 12 healthy controls (6 from the initial microarray analysis and 6 new ones), 12 SOJIA patients, 5 S. aureus, 4 S. pneumoniae, 5 E. coli, and 5 influenza A patients (all from the initial microarray study). FIG. 2C shows that those 8 genes were significantly increased in SOJIA patients (Mann-Whitney test) compared to healthy controls but not in infections compared to healthy controls. FIG. 2D shows the expression of the same genes obtained by microarray analysis. Both patterns of expression were found to be similar.

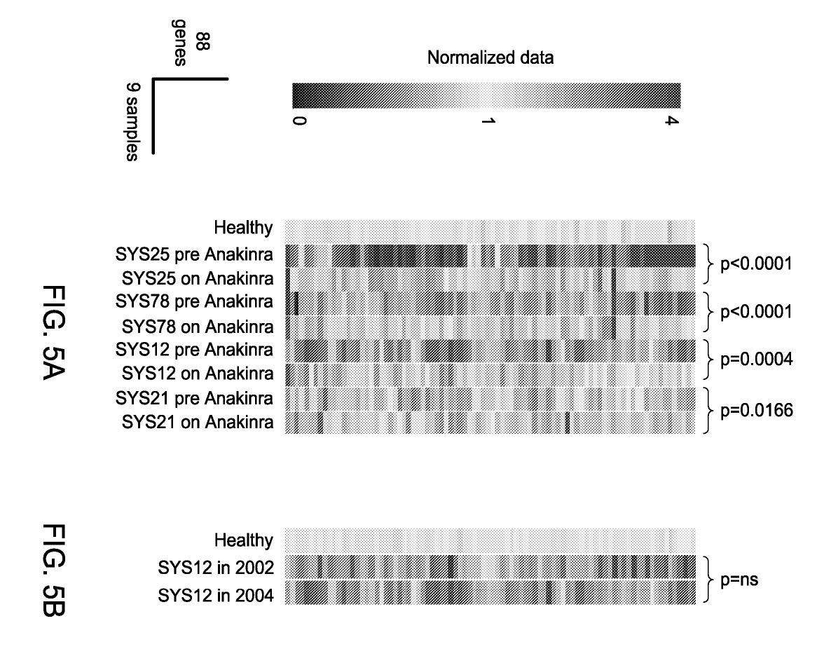

Treatment with IL-Ra (Anakinra) extinguishes the SoJIA-specific signature. The inventors recognized that: (i) serum from SoJIA patients induces IL-1B transcription and protein secretion from healthy PBMCs, and (ii) PBMCs from SoJIA patients display increased production of IL-1B upon activation with PMA-Ionomycin. Accordingly, treatment of SoJIA patients with IL-1Ra results in a dramatic clinical and laboratory response in the majority of patients (9). To test the specificity of the signature obtained through meta-analysis, the expression of the above described 88 genes (Table II) in 4 patients was compared prior to initiation of treatment and 8 weeks after daily subcutaneous injection of IL-1Ra (50-100 mg). In all four patients a statistically significant change was observed in the gene expression levels, with a clear trend towards the values seen in healthy controls (FIG. 5A). To rule out the possibility that differences in gene expression were introduced by technical aspects (the patient samples before and after treatment were processed two months apart), the expression of the same 88 genes on two separate samples from one of the patients (SYS 12) was compared before treatment with Anakinra, which were processed two years apart. As FIG. 5B shows, this analysis yielded no statistically significant difference, supporting that the changes pre and post treatment were in fact due to the specific blockade of IL-1.

SoJIA is the only form of JIA in which systemic symptoms precede the appearance of joint inflammation for weeks to years. Because current laboratory tests are non-specific, a major remaining challenge is how to establish the prompt diagnosis of the disease to avoid lengthy hospitalizations and initiate effective therapy. It is demonstrated herein that gene expression patterns in blood leukocytes can be used to diagnose SoJIA during the systemic phase of the disease.

Patients with SoJIA display a very striking pattern of leukocyte gene transcription when compared with healthy controls. These differences, however, could be due to changes in blood cell composition. Active SoJIA patients, for example, display increased platelet and leukocyte numbers compared to healthy controls. As they also have anemia, erythroid precursors are released from the bone marrow into the blood. Indeed, several genes that were significantly upregulated in SoJIA patients are specific to reticulocytes and correlate with the hematocrit levels in the patients (data not shown). Changes in gene transcription levels that are not merely due to changes in cell composition may be therefore difficult to identify.

A remarkable degree of overlap between the SoJIA signatures obtained from this analysis and the signatures from patients suffering from other febrile diseases, especially Gram (+) and Gram (−) bacterial infections. These patients display similar alterations in blood cell numbers,. i.e., leukocytosis, which could be responsible for some of the transcriptional patterns that were observed. Up to ⅓ of the SLE patients in the study express a signature that overlaps with that from patients with SoJIA and infectious diseases. SLE patients however have low leukocyte counts, suggesting that factors other than cell composition must also contribute to these changes. Age, gender and time of day when the blood is drawn have been described to influence blood gene expression patterns (15). Most of the samples were matched for age and gender with controls and with other disease groups (except for E. coli and influenza A patients who were younger). The time of blood sampling was similar for SoJIA, SLE patients and controls, but more variable for infectious disease patients. It is therefore unlikely that a bias had been introduced by this variable.

In spite of the similarities across disease groups, a significance meta-analysis allowed us to establish an accurate differential diagnosis. The advantage of this analysis is that it permits to normalize each disease group to its own matched control group, therefore avoiding biological (i.e. age, gender) or technical (i.e. array runs) confounding factors. Using this approach, a signature was identified that distinguishes SoJIA patients from infectious diseases as well as from SLE patients. Indeed, 12 highly significant genes from this analysis (p<0.0001 in SoJIA and >0.5 in all other groups) permitted an accurate classification of the disease. The highest overlap (6%) was found with S. aureus infections. Dysregulated cytokine production and/or signaling cascades may be shared by these two disease groups. The present inventors had already noted increased IL-1B production in SoJIA (9). S. aureus-induced production of IL-1B could explain some of the common gene expression patterns that are observe in these patients.

The small number of genes was found to significantly discriminate SoJIA patients from all other conditions included in this study should allow the development of multicenter studies to analyze larger numbers of samples from SoJIA patients and patients with any febrile condition included within the diagnosis of “fever of unknown origin.” The availability of specific diagnostic markers should also allow the prompt initiation of specific therapy even before arthritis develops, avoiding the need for additional therapies. Early diagnosis and treatment would also eventually prevent the development of arthritis and subsequent long term disabilities that these patients have until now endured.

Blocking IL-1 is a useful therapy for SoJIA during both the systemic and arthritic phases of the disease, and as shown here, this treatment extinguishes the SoJIA-specific gene signature in 4/4 patients. It would also be useful to design longitudinal studies to assess the value of this type of analysis in predicting response to therapy in a larger cohort of patients.

TABLE IV
874 Bonferroni genes.

| | | | | |
|---|---|---|---|---|
| | | Average | | |
| | | normalized | | |
| | | data in | | |
| Systematic | P-value | SOJIA | Gene Symbol | Gene Title |

Transport

| | | | | |
|---|---|---|---|---|
| 209994_s_at | 8.03E−05 | 0.5 | ABCB1 | ATP-binding cassette, sub-family B |
| | | | | (MDR/TAP), member 1 |
| 233371_at | 8.96E−06 | 20.1 | ABCC13 | ATP-binding cassette, sub-family C |
| | | | | (CFTR/MRP), member 13 |
| 201613_s_at | 0.000528 | 0.6 | AP1G2 | adaptor-related protein complex 1, gamma 2 |
| | | | | subunit |
| 223237_x_at | 8.07E−05 | 4.2 | AP2A1 | adaptor-related protein complex 2, alpha 1 |
| | | | | subunit |
| 205568_at | 1.61E−05 | 5.1 | AQP9 | aquaporin 9 |
| 208764_s_at | 0.000214 | 0.6 | ATP5G2 | ATP synthase, H+ transporting, mitochondrial |
| | | | | F0 complex, subunit c (subunit 9), isoform 2 |
| 200818_at | 0.00806 | 0.7 | ATP5O | ATP synthase, H+ transporting, mitochondrial |
| | | | | F1 complex, O subunit (oligomycin sensitivity |
| | | | | conferring protein) |
| 212383_at | 0.00803 | 1.9 | ATP6V0A1 | ATPase, H+ transporting, lysosomal V0 |
| | | | | subunit a isoform 1 |
| 208898_at | 0.000528 | 1.7 | ATP6V1D | ATPase, H+ transporting, lysosomal 34 kDa, |
| | | | | V1 subunit D |
| 243615_at | 0.00566 | 1.7 | ATP9B | ATPase, Class II, type 9B |
| 223649_s_at | 1.30E−06 | 12.0 | CGI-69* | CGI-69 protein |
| 213415_at | 0.000214 | 8.3 | CLIC2 | chloride intracellular channel 2 |
| 209143_s_at | 0.0027 | 0.7 | CLNS1A | chloride channel, nucleotide-sensitive, 1A |
| 223450_s_at | 0.000214 | 0.6 | COG3 | component of oligomeric golgi complex 3 |
| 201134_x_at | 0.000214 | 0.5 | COX7C | cytochrome c oxidase subunit VIIc |
| 217491_x_at | 0.00393 | 0.5 | COX7C | cytochrome c oxidase subunit VIIc |
| 229588_at | 0.000337 | 0.7 | DNAJC10 | DnaJ (Hsp40) homolog, subfamily C, member |
| | | | | 10 |
| 209046_s_at | 2.82E−05 | 2.6 | GABARAPL2 | GABA(A) receptor-associated protein-like 2 |
| 210119_at | 0.000214 | 7.2 | KCNJ15 | potassium inwardly-rectifying channel, |
| | | | | subfamily J, member 15 |
| 227934_at | 0.00269 | 0.5 | KPNA5 | Karyopherin alpha 5 (importin alpha 6) |
| 228841_at | 0.00566 | 0.6 | LOC90624 | hypothetical protein LOC90624 |
| 201412_at | 2.56E−06 | 1.6 | LRP10 | low density lipoprotein receptor-related |
| | | | | protein 10 |
| 225008_at | 0.00123 | 2.1 | MGC34646 | Hypothetical protein MGC34646 |
| 212472_at | 0.000214 | 5.0 | MICAL2 | flavoprotein oxidoreductase MICAL2 |
| 212473_s_at | 1.61E−05 | 4.4 | MICAL2 | flavoprotein oxidoreductase MICAL2 |
| 218136_s_at | 1.30E−06 | 8.1 | MSCP | mitochondrial solute carrier protein |
| 218978_s_at | 4.86E−06 | 7.5 | MSCP | mitochondrial solute carrier protein |
| 221920_s_at | 1.30E−06 | 16.0 | MSCP* | mitochondrial solute carrier protein |
| 222528_s_at | 1.30E−06 | 24.3 | MSCP* | mitochondrial solute carrier protein |
| 222529_at | 0.000133 | 10.3 | MSCP | mitochondrial solute carrier protein |
| 231078_at | 1.30E−06 | 25.8 | MSCP | Mitochondrial solute carrier protein |
| 206491_s_at | 0.00123 | 1.6 | NAPA | N-ethylmaleimide-sensitive factor attachment |
| | | | | protein, alpha |
| 205147_x_at | 0.00566 | 1.9 | NCF4 | neutrophil cytosolic factor 4, 40 kDa |
| 207677_s_at | 0.00393 | 2.5 | NCF4 | neutrophil cytosolic factor 4, 40 kDa /// |
| | | | | neutrophil cytosolic factor 4, 40 kDa |
| 201226_at | 0.00123 | 0.6 | NDUFB8 | NADH dehydrogenase (ubiquinone) 1 beta |
| | | | | subcomplex, 8, 19 kDa |
| 200063_s_at | 2.56E−06 | 0.5 | NPM1 | nucleophosmin (nucleolar phosphoprotein |
| | | | | B23, numatrin) |
| 223432_at | 1.30E−06 | 10.9 | OSBP2 | oxysterol binding protein 2 |
| 218047_at | 0.00806 | 0.7 | OSBPL9 | oxysterol binding protein-like 9 |
| 218676_s_at | 8.07E−05 | 3.5 | PCTP | phosphatidylcholine transfer protein |
| 202880_s_at | 0.00183 | 0.7 | PSCD1 | pleckstrin homology, Sec7 and coiled-coil |
| | | | | domains 1(cytohesin 1) |
| 225074_at | 1.30E−06 | 3.5 | RAB2B* | RAB2B, member RAS oncogene family |
| 203582_s_at | 0.00806 | 0.6 | RAB4A | RAB4A, member RAS oncogene family |
| 221808_at | 0.00183 | 0.7 | RAB9A | RAB9A, member RAS oncogene family |
| 202845_s_at | 0.00081 | 2.3 | RALBP1 | ralA binding protein 1 |
| 228548_at | 0.00806 | 0.8 | RAP1A | RAP1A, member of RAS oncogene family |
| 227366_at | 2.82E−05 | 2.3 | RILP | Rab interacting lysosomal protein |
| 206196_s_at | 0.0027 | 3.2 | RPIP8 | RaP2 interacting protein 8 |
| 208456_s_at | 8.03E−05 | 0.5 | RRAS2 | related RAS viral (r-ras) oncogene homolog 2 |
| 202084_s_at | 0.000339 | 1.7 | SEC14L1 | SEC14-like 1 (S. cerevisiae) |
| 202798_at | 0.000339 | 0.6 | SEC24B | SEC24 related gene family, member B (S. cerevisiae) |
| 215009_s_at | 0.000528 | 0.5 | SEC31L1 | SEC31-like 1 (S. cerevisiae) |
| 219349_s_at | 0.00081 | 0.6 | SEC5L1 | SEC5-like 1 (S. cerevisiae) |
| 205856_at | 0.000337 | 8.3 | SLC14A1 | solute carrier family 14 (urea transporter), |
| | | | | member 1 (Kidd blood group) |
| 229151_at | 0.00123 | 8.7 | SLC14A1 | Solute carrier family 14 (urea transporter), |
| | | | | member 1 (Kidd blood group) |
| 205896_at | 1.30E−06 | 5.2 | SLC22A4 | solute carrier family 22 (organic cation |
| | | | | transporter), member 4 |
| 231625_at | 0.00122 | 1.7 | SLC22A9 | solute carrier family 22 (organic anion/cation |
| | | | | transporter), member 9 |
| 202433_at | 0.00566 | 1.4 | SLC35B1 | solute carrier family 35, member B1 |
| 218237_s_at | 0.0027 | 0.6 | SLC38A1 | solute carrier family 38, member 1 |
| 205592_at | 1.30E−06 | 64.2 | SLC4A1 | Solute carrier family 4, anion exchanger, |
| | | | | member 1 (erythrocyte membrane protein |
| | | | | band 3, Diego blood group) |
| 210854_x_at | 1.30E−06 | 9.2 | SLC6A8 | solute carrier family 6 (neurotransmitter |
| | | | | transporter, creatine), member 8 |
| 213843_x_at | 4.86E−06 | 10.2 | SLC6A8 | solute carrier family 6 (neurotransmitter |
| | | | | transporter, creatine), member 8 |
| 210357_s_at | 0.000528 | 6.9 | SMOX | spermine oxidase |
| 208781_x_at | 0.0027 | 2.5 | SNX3 | sorting nexin 3 |
| 210648_x_at | 0.00806 | 1.9 | SNX3 | sorting nexin 3 |
| 213545_x_at | 0.00566 | 2.1 | SNX3 | sorting nexin 3 |
| 212807_s_at | 0.00183 | 2.0 | SORT1 | sortilin 1 |
| 209367_at | 0.000133 | 2.3 | STXBP2 | syntaxin binding protein 2 |
| 201260_s_at | 0.00806 | 0.7 | SYPL | synaptophysin-like protein |
| 218188_s_at | 0.000528 | 0.6 | TIMM13 | translocase of inner mitochondrial membrane |
| | | | | 13 homolog (yeast) |
| 201812_s_at | 0.00183 | 0.6 | TOMM7 | translocase of outer mitochondrial membrane |
| | | | | 7 homolog (yeast) /// hypothetical protein |
| | | | | LOC201725 |
| 205708_s_at | 0.00806 | 2.3 | TRPM2 | transient receptor potential cation channel, |
| | | | | subfamily M, member 2 |
| 205849_s_at | 0.000339 | 0.7 | UQCRB | ubiquinol-cytochrome c reductase binding |
| | | | | protein |
| 209066_x_at | 0.000133 | 0.5 | UQCRB | ubiquinol-cytochrome c reductase binding |
| | | | | protein |
| 209452_s_at | 0.00566 | 1.9 | VTI1B | vesicle transport through interaction with t- |
| | | | | SNAREs homolog 1B (yeast) |
| 206698_at | 0.000214 | 7.1 | XK | Kell blood group precursor (McLeod |
| | | | | phenotype) |

Ubiquitin

| | | | | |
|---|---|---|---|---|
| 210075_at | 0.000807 | 2.6 | 2-3 | membrane-associated ring finger (C3HC4) 2 |
| 221824_s_at | 4.82E−05 | 6.5 | 8-3 | membrane-associated ring finger (C3HC4) 8 |
| 231933_at | 4.86E−06 | 5.1 | 8-3 | membrane-associated ring finger (C3HC4) 8 |
| 202268_s_at | 0.000214 | 0.7 | APPBP1 | amyloid beta precursor protein binding |
| | | | | protein 1, 59 kDa |
| 204190_at | 0.000528 | 0.6 | C13orf22 | chromosome 13 open reading frame 22 |
| 217988_at | 1.30E−06 | 0.5 | CCNB1IP1 | cyclin B1 interacting protein 1 |
| 212540_at | 1.30E−06 | 3.8 | CDC34* | cell division cycle 34 |
| 207231_at | 0.00123 | 0.6 | DZIP3 | zinc finger DAZ interacting protein 3 |
| 213186_at | 0.0027 | 0.6 | DZIP3 | zinc finger DAZ interacting protein 3 |
| 201178_at | 1.30E−06 | 5.3 | FBXO7 | F-box protein 7 |
| 210638_s_at | 1.30E−06 | 2.2 | FBXO9* | F-box protein 9 |
| 218373_at | 4.82E−05 | 0.5 | FTS | fused toes homolog (mouse) |
| 239101_at | 0.00183 | 0.4 | ITCH | itchy homolog E3 ubiquitin protein ligase |
| | | | | (mouse) |
| 209845_at | 2.56E−06 | 4.2 | MKRN1 | makorin, ring finger protein, 1 |
| 201285_at | 1.30E−06 | 3.8 | MKRN1* | makorin, ring finger protein, 1 /// makorin, ring |
| | | | | finger protein, 1 |
| 202219_at | 1.30E−06 | 78.3 | SLC6A8* | solute carrier family 6 (neurotransmitter |
| | | | | transporter, creatine), member 8 |
| 227935_s_at | 0.00183 | 3.2 | PCGF5 | polycomb group ring finger 5 |
| 216088_s_at | 4.86E−06 | 0.6 | PSMA7 | proteasome (prosome, macropain) subunit, |
| | | | | alpha type, 7 |
| 201052_s_at | 0.00393 | 3.4 | PSMF1 | proteasome (prosome, macropain) inhibitor |
| | | | | subunit 1 (PI31) |
| 218247_s_at | 0.00806 | 0.5 | RKHD2 | ring finger and KH domain containing 2 |
| 207801_s_at | 4.86E−06 | 5.4 | RNF10 | ring finger protein 10 |
| 208632_at | 0.000133 | 3.2 | RNF10 | ring finger protein 10 /// ring finger protein 10 |
| 221063_x_at | 8.07E−05 | 3.1 | RNF123 | ring finger protein 123 |
| 202318_s_at | 0.00183 | 0.7 | SENP6 | SUMO1/sentrin specific protease 6 |
| 214790_at | 4.79E−05 | 0.5 | SENP6 | SUMO1/sentrin specific protease 6 |
| 226366_at | 0.00806 | 0.7 | SHPRH | SNF2 histone linker PHD RING helicase |
| 209339_at | 1.30E−06 | 9.0 | SIAH2* | seven in absentia homolog 2 (Drosophila) /// |
| | | | | seven in absentia homolog 2 (Drosophila) |
| 232665_x_at | 0.000133 | 5.1 | SMURF1 | SMAD specific E3 ubiquitin protein ligase 1 |
| 215047_at | 1.30E−06 | 37.9 | TRIM58 | tripartite motif-containing 58 |
| 208661_s_at | 8.07E−05 | 0.5 | TTC3 | tetratricopeptide repeat domain 3 |
| 208662_s_at | 0.00081 | 0.6 | TTC3 | tetratricopeptide repeat domain 3 |
| 222420_s_at | 0.00393 | 4.2 | UBE2H | ubiquitin-conjugating enzyme E2H (UBC8 |
| | | | | homolog, yeast) |
| 201534_s_at | 0.00081 | 0.4 | UBL3 | ubiquitin-like 3 |
| 223117_s_at | 0.000528 | 0.7 | USP47 | ubiquitin specific protease 47 |
| 211678_s_at | 2.82E−05 | 0.6 | ZNF313 | zinc finger protein 313 |

Heme/Hemoglobin

| | | | | |
|---|---|---|---|---|
| 203115_at | 4.86E−06 | 9.8 | FECH | ferrochelatase (protoporphyria) |
| 203116_s_at | 1.30E−06 | 6.6 | FECH* | ferrochelatase (protoporphyria) |
| 211699_x_at | 0.00566 | 3.4 | HBA1 /// HBA2 | hemoglobin, alpha 1 /// hemoglobin, alpha 2 |
| 206834_at | 1.30E−06 | 36.4 | HBD* | hemoglobin, delta /// hemoglobin, delta |
| 204848_x_at | 1.30E−06 | 65.7 | HBG1 /// HBG2 | hemoglobin, gamma A /// hemoglobin, |
| | | | | gamma G |
| 204419_x_at | 1.30E−06 | 48.9 | HBG2 | hemoglobin, gamma G |
| 213515_x_at | 2.56E−06 | 22.0 | HBG2 | hemoglobin, gamma G |
| 240336_at | 1.30E−06 | 161.6 | HBM | hemoglobin mu chain |

Immune Response

| | | | | |
|---|---|---|---|---|
| 205098_at | 0.000133 | 2.6 | CCR1 | chemokine (C—C motif) receptor 1 |
| 205297_s_at | 0.0027 | 0.5 | CD79B | CD79B antigen (immunoglobulin-associated |
| | | | | beta) |
| 209498_at | 4.82E−05 | 8.1 | CEACAM1 | carcinoembryonic antigen-related cell |
| | | | | adhesion molecule 1 (biliary glycoprotein) |
| 211883_x_at | 0.00806 | 3.1 | CEACAM1 | carcinoembryonic antigen-related cell |
| | | | | adhesion molecule 1 (biliary glycoprotein) |
| 239205_s_at | 0.00183 | 4.5 | CR1 | complement component (3b/4b) receptor 1, |
| | | | | including Knops blood group system /// |
| | | | | complement component (3b/4b) receptor 1- |
| | | | | like |
| 204470_at | 0.00183 | 3.1 | CXCL1 | chemokine (C—X—C motif) ligand 1 (melanoma |
| | | | | growth stimulating activity, alpha) |
| 211734_s_at | 4.86E−06 | 0.3 | FCER1A | Fc fragment of IgE, high affinity I, receptor for; |
| | | | | alpha polypeptide /// Fc fragment of IgE, high |
| | | | | affinity I, receptor for; alpha polypeptide |
| 214511_x_at | 1.30E−06 | 4.1 | FCGR1A | Fc fragment of IgG, high affinity Ia, receptor |
| | | | | (CD64) /// Fc-gamma receptor I B2 |
| 216950_s_at | 1.30E−06 | 4.2 | FCGR1A* | Fc fragment of IgG, high affinity Ia, receptor |
| | | | | (CD64) |
| 203932_at | 1.30E−06 | 0.5 | HLA-DMB | major histocompatibility complex, class II, DM |
| | | | | beta |
| 208894_at | 0.0027 | 0.6 | HLA-DRA | major histocompatibility complex, class II, DR |
| | | | | alpha |
| 210982_s_at | 0.00393 | 0.7 | HLA-DRA | major histocompatibility complex, class II, DR |
| | | | | alpha |
| 217456_x_at | 1.30E−06 | 0.6 | HLA-E* | major histocompatibility complex, class I, E |
| 204806_x_at | 1.30E−06 | 0.6 | HLA-F* | major histocompatibility complex, class I, F |
| 221875_x_at | 0.00566 | 0.8 | HLA-F | major histocompatibility complex, class I, F |
| 210514_x_at | 2.82E−05 | 0.3 | HLA-G | HLA-G histocompatibility antigen, class I, G |
| 202411_at | 0.000339 | 15.7 | IFI27 | interferon, alpha-inducible protein 27 |
| 212657_s_at | 1.30E−06 | 3.2 | IL1RN* | interleukin 1 receptor antagonist |
| 206881_s_at | 0.00806 | 2.8 | LILRA3 | leukocyte immunoglobulin-like receptor, |
| | | | | subfamily A (without TM domain), member 3 |
| 210784_x_at | 0.0027 | 1.8 | LILRB3 | leukocyte immunoglobulin-like receptor, |
| | | | | subfamily B (with TM and ITIM domains), |
| | | | | member 3 |
| 211133_x_at | 0.00806 | 1.9 | LILRB3 | leukocyte immunoglobulin-like receptor, |
| | | | | subfamily B (with TM and ITIM domains), |
| | | | | member 3 |
| 211135_x_at | 4.82E−05 | 2.2 | LILRB3 | leukocyte immunoglobulin-like receptor, |
| | | | | subfamily B (with TM and ITIM domains), |
| | | | | member 3 |
| 207339_s_at | 8.07E−05 | 0.5 | LTB | lymphotoxin beta (TNF superfamily, member |
| | | | | 3) |
| 206584_at | 2.56E−06 | 2.1 | LY96 | lymphocyte antigen 96 |
| 243099_at | 4.86E−06 | 1.9 | NFAM1 | NFAT activating protein with ITAM motif 1 |
| 205863_at | 0.00183 | 3.2 | S100A12 | S100 calcium binding protein A12 |
| | | | | (calgranulin C) |
| 211429_s_at | 0.00183 | 1.6 | SERPINA1 | serine (or cysteine) proteinase inhibitor, clade |
| | | | | A (alpha-1 antiproteinase, antitrypsin), |
| | | | | member 1 |
| 206025_s_at | 0.000133 | 4.4 | TNFAIP6 | tumor necrosis factor, alpha-induced protein 6 |
| 206026_s_at | 0.000133 | 5.1 | TNFAIP6 | tumor necrosis factor, alpha-induced protein 6 |
| 210915_x_at | 8.07E−05 | 0.6 | TRBC1 | T cell receptor beta constant 1 |
| 211796_s_at | 4.82E−05 | 0.6 | TRBC1 | T cell receptor beta constant 1 |
| 213193_x_at | 2.82E−05 | 0.7 | TRBC1 | T cell receptor beta constant 1 /// T cell |
| | | | | receptor beta constant 1 |

Metabolism

| | | | | |
|---|---|---|---|---|
| 214274_s_at | 0.0027 | 0.7 | ACAA1 | acetyl-Coenzyme A acyltransferase 1 |
| | | | | (peroxisomal 3-oxoacyl-Coenzyme A |
| | | | | thiolase) |
| 201963_at | 0.000214 | 4.0 | ACSL1 | acyl-CoA synthetase long-chain family |
| | | | | member 1 |
| 207275_s_at | 4.86E−06 | 5.5 | ACSL1 | acyl-CoA synthetase long-chain family |
| | | | | member 1 |
| 217748_at | 1.30E−06 | 9.3 | ADIPOR1 | adiponectin receptor 1 /// adiponectin |
| | | | | receptor 1 |
| 202912_at | 1.61E−05 | 6.6 | ADM | adrenomedullin |
| 202144_s_at | 8.07E−05 | 0.7 | ADSL | adenylosuccinate lyase |
| 208498_s_at | 0.000133 | 0.4 | AMY2A | amylase, alpha 2A; pancreatic |
| 221485_at | 0.0027 | 2.2 | B4GALT5 | UDP-Gal: betaGlcNAc beta 1,4- |
| | | | | galactosyltransferase, polypeptide 5 |
| 203502_at | 1.61E−05 | 8.5 | BPGM | 2,3-bisphosphoglycerate mutase /// 2,3- |
| | | | | bisphosphoglycerate mutase |
| 235802_at | 1.30E−06 | 0.4 | C14orf175* | chromosome 14 open reading frame 175 |
| 220751_s_at | 2.82E−05 | 9.0 | C5orf4 | chromosome 5 open reading frame 4 |
| 205950_s_at | 1.30E−06 | 75.6 | CA1* | carbonic anhydrase I |
| 224060_s_at | 4.82E−05 | 0.6 | CGI-30 | CGI-30 protein |
| 208791_at | 0.00123 | 2.5 | CLU | clusterin (complement lysis inhibitor, SP- |
| | | | | 40,40, sulfated glycoprotein 2, testosterone- |
| | | | | repressed prostate message 2, |
| | | | | apolipoprotein J) |
| 209759_s_at | 4.82E−05 | 0.5 | DCI | dodecenoyl-Coenzyme A delta isomerase |
| | | | | (3,2 trans-enoyl-Coenzyme A isomerase) |
| 203302_at | 0.00183 | 0.6 | DCK | deoxycytidine kinase |
| 206335_at | 0.00182 | 1.6 | GALNS | galactosamine (N-acetyl)-6-sulfate sulfatase |
| | | | | (Morquio syndrome, mucopolysaccharidosis |
| | | | | type IVA) |
| 207387_s_at | 0.00183 | 2.5 | GK | glycerol kinase |
| 214430_at | 0.00566 | 1.3 | GLA | galactosidase, alpha |
| 201576_s_at | 0.00081 | 1.5 | GLB1 | galactosidase, beta 1 |
| 204187_at | 1.30E−06 | 18.8 | GMPR | guanosine monophosphate reductase |
| 201554_x_at | 4.82E−05 | 2.7 | GYG | glycogenin |
| 211275_s_at | 8.07E−05 | 2.4 | GYG | glycogenin |
| 200697_at | 0.00123 | 1.9 | HK1 | hexokinase 1 |
| 205936_s_at | 4.86E−06 | 2.8 | HK3 | hexokinase 3 (white cell) |
| 219403_s_at | 0.00393 | 2.1 | HPSE | heparanase |
| 201193_at | 0.00123 | 2.0 | IDH1 | isocitrate dehydrogenase 1 (NADP+), soluble |
| 218847_at | 8.96E−06 | 9.1 | IMP-2 | IGF-II mRNA-binding protein 2 |
| 201892_s_at | 8.96E−06 | 0.5 | IMPDH2 | IMP (inosine monophosphate) |
| | | | | dehydrogenase 2 |
| 202068_s_at | 2.82E−05 | 2.3 | LDLR | low density lipoprotein receptor (familial |
| | | | | hypercholesterolemia) |
| 217956_s_at | 0.00123 | 0.6 | MASA | E-1 enzyme |
| 206522_at | 0.0027 | 11.9 | MGAM | maltase-glucoamylase (alpha-glucosidase) |
| 226214_at | 0.0027 | 2.0 | MIR16 | membrane interacting protein of RGS16 |
| 201695_s_at | 1.61E−05 | 4.1 | NP | nucleoside phosphorylase |
| 218025_s_at | 4.82E−05 | 0.7 | PECI | peroxisomal D3,D2-enoyl-CoA isomerase |
| 228499_at | 0.00123 | 1.7 | PFKFB4 | 6-phosphofructo-2-kinase/fructose-2,6- |
| | | | | biphosphatase 4 |
| 203335_at | 2.82E−05 | 0.6 | PHYH | phytanoyl-CoA hydroxylase (Refsum disease) |
| 205570_at | 2.82E−05 | 5.0 | PIP5K2A | phosphatidylinositol-4-phosphate 5-kinase, |
| | | | | type II, alpha |
| 202165_at | 4.82E−05 | 0.5 | PPP1R2 | protein phosphatase 1, regulatory (inhibitor) |
| | | | | subunit 2 |
| 224909_s_at | 0.00183 | 1.7 | PREX1 | phosphatidylinositol 3,4,5-trisphosphate- |
| | | | | dependent RAC exchanger 1 |
| 209503_s_at | 0.00393 | 0.7 | PSMC5 | proteasome (prosome, macropain) 26S |
| | | | | subunit, ATPase, 5 |
| 201195_s_at | 0.00806 | 2.7 | SLC7A5 | solute carrier family 7 (cationic amino acid |
| | | | | transporter, y+ system), member 5 |
| 214835_s_at | 0.000528 | 0.6 | SUCLG2 | succinate-CoA ligase, GDP-forming, beta |
| | | | | subunit |
| 215772_x_at | 0.000339 | 0.6 | SUCLG2 | succinate-CoA ligase, GDP-forming, beta |
| | | | | subunit |
| 203234_at | 0.00081 | 2.3 | UPP1 | uridine phosphorylase 1 |

Transcription

| | | | | |
|---|---|---|---|---|
| 217729_s_at | 4.82E−05 | 0.5 | AES | amino-terminal enhancer of split |
| 215684_s_at | 0.000214 | 4.7 | ASCC2 | activating signal cointegrator 1 complex |
| | | | | subunit 2 |
| 205965_at | 0.00393 | 1.8 | BATF | basic leucine zipper transcription factor, ATF- |
| | | | | like |
| 222891_s_at | 0.00123 | 0.4 | BCL11A | B-cell CLL/lymphoma 11A (zinc finger |
| | | | | protein) |
| 209430_at | 0.0027 | 0.6 | BTAF1 | BTAF1 RNA polymerase II, B-TFIID |
| | | | | transcription factor-associated, 170 kDa (Mot1 |
| | | | | homolog, S. cerevisiae) |
| 216305_s_at | 0.000339 | 0.6 | C2orf3 | chromosome 2 open reading frame 3 |
| 202163_s_at | 0.000132 | 0.7 | CNOT8 | CCR4-NOT transcription complex, subunit 8 |
| 237819_at | 2.56E−06 | 2.8 | CREB3L2 | CAMP responsive element binding protein 3- |
| | | | | like 2 |
| 232555_at | 0.00183 | 3.1 | CREB5 | CAMP responsive element binding protein 5 |
| 201160_s_at | 1.30E−06 | 4.9 | CSDA | cold shock domain protein A |
| 201161_s_at | 1.30E−06 | 9.1 | CSDA | cold shock domain protein A |
| 202521_at | 0.00392 | 0.7 | CTCF | CCCTC-binding factor (zinc finger protein) |
| 239083_at | 0.00566 | 0.4 | DKFZp762I137 | hypothetical protein DKFZp762I137 |
| 203624_at | 7.99E−05 | 0.7 | DXYS155E | DNA segment on chromosome X and Y |
| | | | | (unique) 155 expressed sequence |
| 217736_s_at | 0.000214 | 2.7 | EIF2AK1 | eukaryotic translation initiation factor 2-alpha |
| | | | | kinase 1 |
| 232909_s_at | 0.00183 | 0.6 | FALZ | fetal Alzheimer antigen |
| 220760_x_at | 8.96E−06 | 0.6 | FLJ14345 | hypothetical protein FLJ14345 |
| 206583_at | 0.000214 | 0.6 | FLJ20344 | hypothetical protein FLJ20344 |
| 206015_s_at | 0.000214 | 0.6 | FOXJ3 | forkhead box J3 |
| 204131_s_at | 0.00806 | 5.0 | FOXO3A | forkhead box O3A |
| 204132_s_at | 0.00183 | 5.3 | FOXO3A | forkhead box O3A |
| 224891_at | 0.00183 | 4.3 | FOXO3A | forkhead box O3A |
| 223287_s_at | 0.000133 | 0.4 | FOXP1 | forkhead box P1 |
| 224838_at | 0.000528 | 0.6 | FOXP1 | forkhead box P1 |
| 209604_s_at | 2.82E−05 | 0.5 | GATA3 | GATA binding protein 3 |
| 229394_s_at | 0.00081 | 0.6 | GRLF1 | Glucocorticoid receptor DNA binding factor 1 |
| 201209_at | 2.56E−06 | 0.5 | HDAC1 | histone deacetylase 1 |
| 214438_at | 0.00806 | 2.6 | HLX1 | H2.0-like homeo box 1 (Drosophila) |
| 205070_at | 0.000339 | 0.6 | ING3 | inhibitor of growth family, member 3 |
| 55872_at | 0.00123 | 0.5 | KIAA1196 | KIAA1196 protein |
| 210504_at | 1.30E−06 | 6.7 | KLF1 | Kruppel-like factor 1 (erythroid) |
| 227198_at | 0.00393 | 0.4 | LAF4 | Lymphoid nuclear protein related to AF4 |
| 226275_at | 0.000339 | 2.6 | MAD | MAX dimerization protein 1 |
| 228846_at | 0.000133 | 3.4 | MAD | MAX dimerization protein 1 |
| 218438_s_at | 0.000214 | 0.6 | MED28 | mediator of RNA polymerase II transcription, |
| | | | | subunit 28 homolog (yeast) |
| 225159_s_at | 0.00183 | 0.7 | MED28 | Mediator of RNA polymerase II transcription, |
| | | | | subunit 28 homolog (yeast) |
| 238761_at | 8.96E−06 | 0.4 | MED28 | Mediator of RNA polymerase II transcription, |
| | | | | subunit 28 homolog (yeast) |
| 217843_s_at | 0.00806 | 0.6 | MED4 | mediator of RNA polymerase II transcription, |
| | | | | subunit 4 homolog (yeast) |
| 218259_at | 0.00566 | 0.6 | MKL2 | MKL/myocardin-like 2 |
| 223189_x_at | 4.82E−05 | 0.5 | MLL5 | myeloid/lymphoid or mixed-lineage leukemia |
| | | | | 5 (trithorax homolog, Drosophila) |
| 223190_s_at | 0.00566 | 0.5 | MLL5 | myeloid/lymphoid or mixed-lineage leukemia |
| | | | | 5 (trithorax homolog, Drosophila) |
| 226100_at | 0.00806 | 0.5 | MLL5 | myeloid/lymphoid or mixed-lineage leukemia |
| | | | | 5 (trithorax homolog, Drosophila) |
| 202364_at | 1.30E−06 | 5.8 | MXI1 | MAX interactor 1 /// MAX interactor 1 |
| 209930_s_at | 1.30E−06 | 6.4 | NFE2 | nuclear factor (erythroid-derived 2), 45 kDa |
| 225768_at | 0.000339 | 0.6 | NR1D2 | nuclear receptor subfamily 1, group D, |
| | | | | member 2 |
| 228569_at | 2.82E−05 | 0.6 | PAPOLA | poly(A) polymerase alpha |
| 239210_at | 0.00123 | 4.3 | PBX1 | Pre-B-cell leukemia transcription factor 1 |
| 212259_s_at | 0.000528 | 0.5 | PBXIP1 | pre-B-cell leukemia transcription factor |
| | | | | interacting protein 1 |
| 214177_s_at | 1.61E−05 | 0.6 | PBXIP1 | pre-B-cell leukemia transcription factor |
| | | | | interacting protein 1 |
| 212660_at | 0.00393 | 0.6 | PHF15 | PHD finger protein 15 |
| 209422_at | 8.96E−06 | 0.5 | PHF20 | PHD finger protein 20 |
| 217952_x_at | 4.86E−06 | 0.7 | PHF3 | PHD finger protein 3 |
| 217864_s_at | 1.61E−05 | 0.6 | PIAS1 | protein inhibitor of activated STAT, 1 |
| 204839_at | 0.0027 | 0.7 | POP5 | processing of precursor 5, ribonuclease |
| | | | | P/MRP subunit (S. cerevisiae) |
| 203497_at | 0.000528 | 0.5 | PPARBP | PPAR binding protein |
| 232517_s_at | 1.61E−05 | 2.9 | PRIC285 | peroxisomal proliferator-activated receptor A |
| | | | | interacting complex 285 |
| 212332_at | 0.00566 | 0.5 | RBL2 | retinoblastoma-like 2 (p130) |
| 219286_s_at | 0.00183 | 0.6 | RBM15 | RNA binding motif protein 15 |
| 204633_s_at | 0.000339 | 0.6 | RPS6KA5 | ribosomal protein S6 kinase, 90 kDa, |
| | | | | polypeptide 5 |
| 222514_at | 0.000994 | 1.4 | RRAGC | Ras-related GTP binding C |
| 202426_s_at | 0.0027 | 3.0 | RXRA | retinoid X receptor, alpha |
| 208740_at | 4.82E−05 | 0.5 | SAP18 | sin3-associated polypeptide, 18 kDa |
| 203408_s_at | 0.00123 | 0.7 | SATB1 | special AT-rich sequence binding protein 1 |
| | | | | (binds to nuclear matrix/scaffold-associating |
| | | | | DNA's) |
| 203077_s_at | 0.00269 | 0.7 | SMAD2 | SMAD, mothers against DPP homolog 2 |
| | | | | (Drosophila) |
| 235725_at | 0.000339 | 0.6 | SMAD4 | SMAD, mothers against DPP homolog 4 |
| | | | | (Drosophila) |
| 201417_at | 8.96E−06 | 0.5 | SOX4 | SRY (sex determining region Y)-box 4 |
| 201139_s_at | 0.000214 | 0.6 | SSB | Sjogren syndrome antigen B (autoantigen La) |
| 203787_at | 0.00183 | 0.6 | SSBP2 | single-stranded DNA binding protein 2 |
| 209358_at | 0.00392 | 0.6 | TAF11 | TAF11 RNA polymerase II, TATA box binding |
| | | | | protein (TBP)-associated factor, 28 kDa |
| 216925_s_at | 1.30E−06 | 17.2 | TAL1 | T-cell acute lymphocytic leukemia 1 |
| 203753_at | 0.00806 | 0.5 | TCF4 | transcription factor 4 |
| 213891_s_at | 0.00566 | 0.5 | TCF4 | Transcription factor 4 |
| 205255_x_at | 0.000214 | 0.6 | TCF7 | transcription factor 7 (T-cell specific, HMG- |
| | | | | box) |
| 206649_s_at | 0.0027 | 1.9 | TFE3 | transcription factor binding to IGHM enhancer 3 |
| 206472_s_at | 0.000214 | 2.0 | TLE3 | transducin-like enhancer of split 3 (E(sp1) |
| | | | | homolog, Drosophila) |
| 217501_at | 0.000528 | 0.5 | WDR39 | WD repeat domain 39 |
| 202979_s_at | 0.00566 | 0.6 | ZF | HCF-binding transcription factor Zhangfei |
| 227796_at | 0.00123 | 0.6 | ZFP62 | zinc finger protein 62 homolog (mouse) |
| 225221_at | 0.00183 | 0.5 | ZKSCAN1 | Zinc finger with KRAB and SCAN domains 1 |
| 202136_at | 0.00806 | 0.7 | ZMYND11 | zinc finger, MYND domain containing 11 |
| 219571_s_at | 0.0027 | 0.5 | ZNF12 /// | zinc finger protein 12 (KOX 3) /// zinc finger |
| | | | ZNF325 | protein 325 |
| 221873_at | 8.07E−05 | 0.6 | ZNF143 | zinc finger protein 143 (clone pHZ-1) |
| 214686_at | 0.00123 | 0.6 | ZNF266 | zinc finger protein 266 |
| 218490_s_at | 0.00806 | 0.5 | ZNF302 | zinc finger protein 302 |
| 228392_at | 0.00123 | 0.5 | ZNF302 | Zinc finger protein 302 |
| 215359_x_at | 0.00393 | 0.6 | ZNF44 | zinc finger protein 44 (KOX 7) |
| 228138_at | 0.000214 | 0.7 | ZNF498 | zinc finger protein 498 |
| 223392_s_at | 0.00393 | 1.8 | ZNF537 | zinc finger protein 537 |
| 218735_s_at | 0.00806 | 0.7 | ZNF544 | zinc finger protein 544 |
| 211721_s_at | 0.00183 | 0.6 | ZNF551 | zinc finger protein 551 /// zinc finger protein |
| | | | | 551 |
| 224518_s_at | 0.00081 | 0.5 | ZNF559 | zinc finger protein 559 /// zinc finger protein |
| | | | | 559 |
| 208137_x_at | 2.82E−05 | 0.5 | ZNF611 | zinc finger protein 611 /// zinc finger protein |
| | | | | 611 |
| 205089_at | 0.00806 | 0.6 | ZNF7 | zinc finger protein 7 (KOX 4, clone HF.16) |
| 221645_s_at | 0.00393 | 0.5 | ZNF83 | zinc finger protein 83 (HPF1) |
| 206158_s_at | 0.00123 | 0.7 | ZNF9 | zinc finger protein 9 (a cellular retroviral |
| | | | | nucleic acid binding protein) |
| 235170_at | 0.00566 | 0.6 | ZNF92 | zinc finger protein 92 (HTF12) |

Extracellular matrix

| | | | | |
|---|---|---|---|---|
| 209840_s_at | 0.000133 | 0.3 | LRRN3 | leucine rich repeat neuronal 3 |
| 209841_s_at | 0.000339 | 0.3 | LRRN3 | leucine rich repeat neuronal 3 |
| 203936_s_at | 0.000214 | 13.3 | MMP9 | matrix metalloproteinase 9 (gelatinase B, |
| | | | | 92 kDa gelatinase, 92 kDa type IV |
| | | | | collagenase) |
| 202524_s_at | 1.61E−05 | 0.6 | SPOCK2 | sparc/osteonectin, cwcv and kazal-like |
| | | | | domains proteoglycan (testican) 2 |

cytoskeleton/microtubule

| | | | | |
|---|---|---|---|---|
| 218395_at | 0.00393 | 0.7 | ACTR6 | ARP6 actin-related protein 6 homolog (yeast) |
| 201753_s_at | 0.00183 | 0.6 | ADD3 | adducin 3 (gamma) |
| 205882_x_at | 0.00393 | 0.7 | ADD3 | adducin 3 (gamma) |
| 205389_s_at | 1.61E−05 | 5.7 | ANK1 | ankyrin 1, erythrocytic /// ankyrin 1, |
| | | | | erythrocytic |
| 208353_x_at | 0.00123 | 5.8 | ANK1 | ankyrin 1, erythrocytic |
| 202391_at | 0.000133 | 3.2 | BASP1 | brain abundant, membrane attached signal |
| | | | | protein 1 |
| 208451_s_at | 0.000528 | 6.8 | C4A /// C4B | complement component 4A /// complement |
| | | | | component 4B /// complement component 4B, |
| | | | | telomeric |
| 201605_x_at | 0.00081 | 1.7 | CNN2 | calponin 2 |
| 218660_at | 1.30E−06 | 4.4 | DYSF* | dysferlin, limb girdle muscular dystrophy 2B |
| | | | | (autosomal recessive) |
| 210746_s_at | 1.30E−06 | 142.5 | EPB42 | erythrocyte membrane protein band 4.2 /// |
| | | | | erythrocyte membrane protein band 4.2 |
| 204505_s_at | 1.30E−06 | 11.5 | EPB49* | erythrocyte membrane protein band 4.9 |
| | | | | (dematin) |
| 207721_x_at | 1.30E−06 | 0.6 | HINT1 * | histidine triad nucleotide binding protein 1 |
| 212878_s_at | 1.30E−06 | 0.6 | KNS2* | kinesin 2 60/70 kDa |
| 205900_at | 4.82E−05 | 19.9 | KRT1 | keratin 1 (epidermolytic hyperkeratosis) |
| 232011_s_at | 1.29E−06 | 2.3 | MAP1LC3A* | microtubule-associated protein 1 light chain 3 |
| | | | | alpha |
| 210088_x_at | 1.30E−06 | 9.9 | MYL4 | myosin, light polypeptide 4, alkali; atrial, |
| | | | | embryonic |
| 216054_x_at | 1.30E−06 | 10.2 | MYL4 | myosin, light polypeptide 4, alkali; atrial, |
| | | | | embryonic |
| 217274_x_at | 2.56E−06 | 5.7 | MYL4 | myosin, light polypeptide 4, alkali; atrial, |
| | | | | embryonic |
| 210395_x_at | 1.30E−06 | 11.6 | MYL4* | myosin, light polypeptide 4, alkali; atrial, |
| | | | | embryonic |
| 226335_at | 0.00123 | 0.7 | RPS6KA3 | ribosomal protein S6 kinase, 90 kDa, |
| | | | | polypeptide 3 |
| 201060_x_at | 4.82E−05 | 3.6 | STOM | stomatin |
| 201061_s_at | 8.07E−05 | 2.7 | STOM | stomatin |
| 203662_s_at | 1.30E−06 | 29.8 | TMOD1 | tropomodulin 1 |
| 203661_s_at | 1.30E−06 | 9.9 | TMOD1* | tropomodulin 1 |
| 210987_x_at | 0.00183 | 2.8 | TPM1 | Tropomyosin 1 (alpha) |
| 212481_s_at | 0.00183 | 1.7 | TPM4 | tropomyosin 4 |
| 219351_at | 2.82E−05 | 0.6 | TRAPPC2 | trafficking protein particle complex 2 |
| 209251_x_at | 0.00183 | 1.4 | TUBA6 | tubulin alpha 6 |
| 210389_x_at | 0.00393 | 0.7 | TUBD1 | tubulin, delta 1 |
| 208623_s_at | 0.000339 | 0.5 | VIL2 | villin 2 (ezrin) |

G-protein coupled receptor

| | | | | |
|---|---|---|---|---|
| 218030_at | 1.30E−06 | 3.8 | GIT1* | G protein-coupled receptor kinase interactor 1 |
| 204000_at | 8.96E−06 | 0.6 | GNB5 | guanine nucleotide binding protein (G |
| | | | | protein), beta 5 |
| 228770_at | 2.82E−05 | 9.8 | GPR146 | G protein-coupled receptor 146 |
| 212090_at | 0.000133 | 2.9 | GRINA | glutamate receptor, ionotropic, N-methyl D- |
| | | | | asparate-associated protein 1 (glutamate |
| | | | | binding) |
| 233657_at | 0.00123 | 1.9 | OPN5 | opsin 5 |
| 224707_at | 4.86E−06 | 5.1 | ORF1-FL49 | putative nuclear protein ORF1-FL49 |
| 201042_at | 4.86E−06 | 5.0 | TGM2 | transglutaminase 2 (C polypeptide, protein- |
| | | | | glutamine-gamma-glutamyltransferase) |

Signal transduction

| | | | | |
|---|---|---|---|---|
| 202096_s_at | 0.000528 | 1.9 | BZRP | benzodiazapine receptor (peripheral) |
| 208826_x_at | 4.82E−05 | 0.7 | HINT1 | histidine triad nucleotide binding protein 1 |
| 227087_at | 0.00183 | 0.5 | INPP4A | Inositol polyphosphate-4-phosphatase, type I, |
| | | | | 107 kDa |
| 202974_at | 1.30E−06 | 7.5 | MPP1 | membrane protein, palmitoylated 1, 55 kDa |
| 219607_s_at | 4.82E−05 | 6.6 | MS4A4A | membrane-spanning 4-domains, subfamily A, |
| | | | | member 4 |
| 222317_at | 0.00803 | 0.5 | PDE3B | Phosphodiesterase 3B, cGMP-inhibited |
| 223358_s_at | 0.00081 | 0.6 | PDE7A | Phosphodiesterase 7A |
| 201877_s_at | 0.00566 | 0.7 | PPP2R5C | protein phosphatase 2, regulatory subunit B |
| | | | | (B56), gamma isoform |
| 229322_at | 0.00566 | 0.7 | PPP2R5E | protein phosphatase 2, regulatory subunit B |
| | | | | (B56), epsilon isoform |

Regulation translation

| | | | | |
|---|---|---|---|---|
| 209861_s_at | 0.000528 | 0.6 | METAP2 | methionyl aminopeptidase 2 |
| 244185_at | 0.00806 | 0.5 | METAP2 | Methionyl aminopeptidase 2 |
| 218205_s_at | 0.00566 | 0.6 | MKNK2 | MAP kinase interacting serine/threonine |
| | | | | kinase 2 |

Cell cycle

| | | | | |
|---|---|---|---|---|
| 201458_s_at | 0.00081 | 0.6 | BUB3 | BUB3 budding uninhibited by benzimidazoles |
| | | | | 3 homolog (yeast) |
| 208796_s_at | 0.00806 | 0.7 | CCNG1 | cyclin G1 |
| 232266_x_at | 0.00123 | 0.4 | CDC2L5 | Cell division cycle 2-like 5 (cholinesterase- |
| | | | | related cell division controller) |
| 209112_at | 8.96E−06 | 0.6 | CDKN1B | cyclin-dependent kinase inhibitor 1B (p27, |
| | | | | Kip1) |
| 2028_s_at | 0.00566 | 1.8 | E2F1 | E2F transcription factor 1 |
| 228361_at | 2.82E−05 | 3.8 | E2F2 | E2F transcription factor 2 |
| 201912_s_at | 1.30E−06 | 6.3 | GSPT1 | G1 to S phase transition 1 /// G1 to S phase |
| | | | | transition 1 |
| 215438_x_at | 4.82E−05 | 6.3 | GSPT1 | G1 to S phase transition 1 |
| 211040_x_at | 0.000133 | 2.0 | GTSE1 | G-2 and S-phase expressed 1 /// G-2 and S- |
| | | | | phase expressed 1 |
| 210212_x_at | 0.00566 | 0.8 | MTCP1 | mature T-cell proliferation 1 |
| 200658_s_at | 0.000214 | 0.5 | PHB | prohibitin |
| 238656_at | 0.0027 | 0.6 | RAD50 | RAD50 homolog (S. cerevisiae) |
| 212783_at | 0.0027 | 0.5 | RBBP6 | retinoblastoma binding protein 6 |
| 203175_at | 4.82E−05 | 1.9 | RHOG | ras homolog gene family, member G (rho G) |
| 235683_at | 1.29E−06 | 19.6 | SESN3* | sestrin 3 |
| 235684_s_at | 4.86E−06 | 7.6 | SESN3 | sestrin 3 |
| 210567_s_at | 0.00081 | 0.7 | SKP2 | S-phase kinase-associated protein 2 (p45) |
| 212330_at | 1.61E−05 | 2.9 | TFDP1 | transcription factor Dp-1 |
| 222243_s_at | 0.00081 | 0.5 | TOB2 | transducer of ERBB2, 2 |

NF-kB

| | | | | |
|---|---|---|---|---|
| 203113_s_at | 1.30E−06 | 0.2 | EEF1D* | eukaryotic translation elongation factor 1 |
| | | | | delta (guanine nucleotide exchange protein) |

protein biosynthesis

| | | | | |
|---|---|---|---|---|
| 204905_s_at | 0.0027 | 0.6 | EEF1E1 | eukaryotic translation elongation factor 1 |
| | | | | epsilon 1 |
| 210501_x_at | 0.00806 | 0.7 | EIF3S12 | eukaryotic translation initiation factor 3, |
| | | | | subunit 12 |
| 221494_x_at | 0.00393 | 0.7 | EIF3S12 | eukaryotic translation initiation factor 3, |
| | | | | subunit 12 |
| 201592_at | 0.0027 | 0.7 | EIF3S3 | eukaryotic translation initiation factor 3, |
| | | | | subunit 3 gamma, 40 kDa |
| 208887_at | 0.000528 | 0.6 | EIF3S4 | eukaryotic translation initiation factor 3, |
| | | | | subunit 4 delta, 44 kDa |
| 212904_at | 4.82E−05 | 0.7 | KIAA1185 | KIAA1185 protein |
| 226588_at | 0.00123 | 0.4 | KIAA1604 | KIAA1604 protein |
| 222064_s_at | 8.96E−06 | 0.7 | MGC2744 | hypothetical protein MGC2744 |
| 224479_s_at | 2.56E−06 | 0.7 | MRPL45 | mitochondrial ribosomal protein L45 |
| 217408_at | 0.00806 | 0.6 | MRPS18B | mitochondrial ribosomal protein S18B |
| 225477_s_at | 0.00393 | 0.6 | MRPS25 | Mitochondrial nbosomal protein S25 |
| 200735_x_at | 0.00081 | 0.6 | NACA | nascent-polypeptide-associated complex |
| | | | | alpha polypeptide |
| 200036_s_at | 0.00393 | 0.7 | RPL10A | ribosomal protein L10a |
| 213588_x_at | 0.000339 | 0.6 | RPL14 | ribosomal protein L14 |
| 200074_s_at | 0.00806 | 0.7 | RPL14 | ribosomal protein L14 |
| 217266_at | 0.0027 | 0.6 | RPL15 | ribosomal protein L15 |
| 221475_s_at | 0.000528 | 0.7 | RPL15 | ribosomal protein L15 |
| 221476_s_at | 2.56E−06 | 0.7 | RPL15 | ribosomal protein L15 |
| 216383_at | 0.00806 | 0.4 | RPL18A | ribosomal protein L18a |
| 214042_s_at | 1.30E−06 | 0.7 | RPL22 | ribosomal protein L22 |
| 221726_at | 1.61E−05 | 0.7 | RPL22 | ribosomal protein L22 |
| 200823_x_at | 0.000339 | 0.6 | RPL29 | ribosomal protein L29 |
| 200002_at | 0.00123 | 0.6 | RPL35 | ribosomal protein L35 |
| 200089_s_at | 4.86E−06 | 0.6 | RPL4 | ribosomal protein L4 |
| 201154_x_at | 8.07E−05 | 0.7 | RPL4 | ribosomal protein L4 |
| 200937_s_at | 8.96E−06 | 0.7 | RPL5 | ribosomal protein L5 |
| 208646_at | 1.30E−06 | 0.4 | RPS14* | ribosomal protein S14 |
| 218007_s_at | 0.000528 | 0.6 | RPS27L | ribosomal protein S27-like |
| 200082_s_at | 0.00123 | 0.7 | RPS7 | ribosomal protein S7 |
| 200858_s_at | 0.00081 | 0.7 | RPS8 | ribosomal protein S8 |
| 214317_x_at | 0.00393 | 0.5 | RPS9 | ribosomal protein S9 |
| 212018_s_at | 4.86E−06 | 0.5 | RSL1D1 | ribosomal L1 domain containing 1 |
| 200802_at | 4.82E−05 | 0.8 | SARS | seryl-tRNA synthetase |
| 202614_at | 0.00806 | 0.7 | SLC30A9 | solute carrier family 30 (zinc transporter), |
| | | | | member 9 |
| 201922_at | 0.0027 | 0.6 | TINP1 | TGF beta-inducible nuclear protein 1 |
| 204703_at | 0.00182 | 0.7 | TTC10 | tetratricopeptide repeat domain 10 |
| 206621_s_at | 0.00183 | 0.7 | WBSCR1 | Williams-Beuren syndrome chromosome |
| | | | | region 1 |

protein amino acid phosphorylation

| | | | | |
|---|---|---|---|---|
| 223266_at | 1.30E−06 | 13.9 | ALS2CR2* | amyotrophic lateral sclerosis 2 (juvenile) |
| | | | | chromosome region, candidate 2 |
| 59644_at | 0.00325 | 2.1 | BMP2K | BMP2 inducible kinase |
| 203468_at | 1.61E−05 | 0.3 | CDK10 | cyclin-dependent kinase (CDC2-like) 10 |
| 227767_at | 0.000339 | 0.6 | CSNK1G3 | Casein kinase 1, gamma 3 |
| 213980_s_at | 4.82E−05 | 0.4 | CTBP1 | C-terminal binding protein 1 |
| 208018_s_at | 0.0027 | 1.5 | HCK | hemopoietic cell kinase |
| 201234_at | 0.00081 | 1.6 | ILK | integrin-linked kinase |
| 227677_at | 0.00393 | 1.7 | JAK3 | Janus kinase 3 (a protein tyrosine kinase, |
| | | | | leukocyte) |
| 204155_s_at | 4.82E−05 | 0.5 | KIAA0999 | KIAA0999 protein |
| 202193_at | 0.0027 | 2.6 | LIMK2 | LIM domain kinase 2 |
| 210582_s_at | 0.00183 | 2.6 | LIMK2 | LIM domain kinase 2 |
| 207667_s_at | 8.96E−06 | 4.5 | MAP2K3 | mitogen-activated protein kinase kinase 3 |
| 215498_s_at | 0.000339 | 4.4 | MAP2K3 | mitogen-activated protein kinase kinase 3 |
| 215499_at | 0.0027 | 4.3 | MAP2K3 | Mitogen-activated protein kinase kinase 3 |
| 202530_at | 8.07E−05 | 2.0 | MAPK14 | mitogen-activated protein kinase 14 |
| 202568_s_at | 2.82E−05 | 2.4 | MARK3 | MAP/microtubule affinity-regulating kinase 3 |
| 218499_at | 0.000528 | 0.6 | MST4 | Mst3 and SOK1-related kinase |
| 41329_at | 0.00081 | 0.6 | PACE-1 | ezrin-binding partner PACE-1 |
| 208875_s_at | 4.82E−05 | 1.5 | PAK2 | p21 (CDKN1A)-activated kinase 2 |
| 209018_s_at | 4.86E−06 | 3.0 | PINK1 | PTEN induced putative kinase 1 |
| 218764_at | 4.86E−06 | 0.5 | PRKCH | protein kinase C, eta |
| 202129_s_at | 4.82E−05 | 5.9 | RIOK3 | RIO kinase 3 (yeast) /// RIO kinase 3 (yeast) |
| 202130_at | 0.00393 | 3.8 | RIOK3 | RIO kinase 3 (yeast) /// RIO kinase 3 (yeast) |
| 202131_s_at | 0.00081 | 3.3 | RIOK3 | RIO kinase 3 (yeast) /// RIO kinase 3 (yeast) |
| 209481_at | 0.00081 | 0.7 | SNRK | SNF-1 related kinase |
| 204062_s_at | 0.00393 | 0.7 | ULK2 | unc-51-like kinase 2 (C. elegans) |

Apoptosis

| | | | | |
|---|---|---|---|---|
| 202512_s_at | 0.00393 | 0.6 | APG5L | APG5 autophagy 5-like (S. cerevisiae) |
| 202387_at | 8.96E−06 | 4.6 | BAG1 | BCL2-associated athanogene |
| 211475_s_at | 1.30E−06 | 3.7 | BAG1* | BCL2-associated athanogene |
| 202985_s_at | 0.00183 | 0.7 | BAG5 | BCL2-associated athanogene 5 |
| 206665_s_at | 2.82E−05 | 17.7 | BCL2L1 | BCL2-like 1 |
| 212312_at | 1.30E−06 | 9.5 | BCL2L1* | BCL2-like 1 |
| 215037_s_at | 1.30E−06 | 6.4 | BCL2L1* | BCL2-like 1 |
| 204861_s_at | 0.00806 | 2.0 | BIRC1 | baculoviral IAP repeat-containing 1 |
| 221479_s_at | 0.000214 | 6.1 | BNIP3L | BCL2/adenovirus E1B 19 kDa interacting |
| | | | | protein 3-like /// BCL2/adenovirus E1B 19 kDa |
| | | | | interacting protein 3-like |
| 200920_s_at | 0.00123 | 0.6 | BTG1 | B-cell translocation gene 1, anti-proliferative |
| 213581_at | 4.82E−05 | 0.6 | PDCD2 | programmed cell death 2 |
| 212594_at | 2.82E−05 | 0.5 | PDCD4 | programmed cell death 4 (neoplastic |
| | | | | transformation inhibitor) |
| 200608_s_at | 0.0027 | 0.7 | RAD21 | RAD21 homolog (S. pombe) |
| 211509_s_at | 0.00183 | 1.4 | RTN4 | reticulon 4 |
| 204466_s_at | 1.30E−06 | 28.0 | SNCA* | synuclein, alpha (non A4 component of |
| | | | | amyloid precursor) /// synuclein, alpha (non |
| | | | | A4 component of amyloid precursor) |
| 204467_s_at | 1.30E−06 | 30.9 | SNCA | synuclein, alpha (non A4 component of |
| | | | | amyloid precursor) /// synuclein, alpha (non |
| | | | | A4 component of amyloid precursor) |
| 207827_x_at | 1.30E−06 | 16.7 | SNCA* | synuclein, alpha (non A4 component of |
| | | | | amyloid precursor) |
| 211546_x_at | 1.30E−06 | 13.4 | SNCA* | synuclein, alpha (non A4 component of |
| | | | | amyloid precursor) |
| 200803_s_at | 0.00806 | 1.4 | TEGT | testis enhanced gene transcript (BAX inhibitor |
| | | | | 1) |
| 221602_s_at | 0.0027 | 0.5 | TOSO | regulator of Fas-induced apoptosis /// |
| | | | | regulator of Fas-induced apoptosis |

proteolysis and peptidolysis

| | | | | |
|---|---|---|---|---|
| 200839_s_at | 4.86E−06 | 2.4 | CTSB | cathepsin B |
| 213274_s_at | 4.82E−05 | 2.2 | CTSB | cathepsin B |
| 201945_at | 0.000339 | 2.2 | FURIN | furin (paired basic amino acid cleaving |
| | | | | enzyme) |
| 207460_at | 1.30E−06 | 0.4 | GZMM | granzyme M (lymphocyte met-ase 1) |
| 206697_s_at | 2.56E−06 | 5.4 | HP | haptoglobin |
| 208470_s_at | 0.00393 | 13.9 | HP | haptoglobin |
| 210017_at | 4.82E−05 | 0.5 | MALT1 | mucosa associated lymphoid tissue |
| | | | | lymphoma translocation gene 1 |
| 210018_x_at | 0.00123 | 0.6 | MALT1 | mucosa associated lymphoid tissue |
| | | | | lymphoma translocation gene 1 |
| 207890_s_at | 0.000214 | 2.7 | MMP25 | matrix metalloproteinase 25 |
| 208709_s_at | 0.00123 | 1.5 | NRD1 | nardilysin (N-arginine dibasic convertase) |
| 200661_at | 8.07E−05 | 1.7 | PPGB | protective protein for beta-galactosiase |
| | | | | (galactosialidosis) |

cell growth

| | | | | |
|---|---|---|---|---|
| 221675_s_at | 4.86E−06 | 5.6 | CHPT1 | choline phosphotransferase 1 |
| 206359_at | 0.00123 | 4.7 | SOCS3 | suppressor of cytokine signaling 3 |
| 227697_at | 0.000339 | 5.5 | SOCS3 | suppressor of cytokine signaling 3 |
| 201758_at | 2.56E−06 | 1.7 | TSG101 | tumor susceptibility gene 101 |

protein folding

| | | | | |
|---|---|---|---|---|
| 218168_s_at | 0.00806 | 0.7 | CABC1 | chaperone, ABC1 activity of bc1 complex like |
| | | | | (S. pombe) |
| 209275_s_at | 0.00393 | 1.7 | CLN3 | ceroid-lipofuscinosis, neuronal 3, juvenile |
| | | | | (Batten, Spielmeyer-Vogt disease) |
| 228622_s_at | 0.00393 | 0.7 | DNAJC4 | DnaJ (Hsp40) homolog, subfamily C, member 4 |
| 219672_at | 1.30E−06 | 30.0 | ERAF | erythroid associated factor |
| 229949_at | 0.00123 | 0.6 | FKBP6 | FK506 binding protein 6, 36 kDa |
| 40850_at | 1.30E−06 | 48.4 | FKBP8* | FK506 binding protein 8, 38 kDa |
| 205361_s_at | 0.00183 | 0.6 | PFDN4 | prefoldin 4 |
| 201759_at | 0.00806 | 2.2 | TBCD | tubulin-specific chaperone d |
| 200810_s_at | 8.96E−06 | 0.5 | CIRBP | cold inducible RNA binding protein |
| 200811_at | 0.00806 | 0.6 | CIRBP | cold inducible RNA binding protein |
| 211938_at | 8.96E−06 | 0.6 | EIF4B | eukaryotic translation initiation factor 4B |
| 214280_x_at | 0.00393 | 0.6 | HNRPA1 | heterogeneous nuclear ribonucleoprotein A1 |
| 201993_x_at | 1.30E−06 | 0.6 | HNRPDL | heterogeneous nuclear ribonucleoprotein D- |
| | | | | like |
| 209067_s_at | 1.61E−05 | 0.5 | HNRPDL | heterogeneous nuclear ribonucleoprotein D- |
| | | | | like |
| 209068_at | 1.30E−06 | 0.5 | HNRPDL | heterogeneous nuclear ribonucleoprotein D- |
| | | | | like |
| 225394_s_at | 0.00566 | 0.6 | MADP-1 | MADP-1 protein |
| 210093_s_at | 1.61E−05 | 0.3 | MAGOH | mago-nashi homolog, proliferation-associated |
| | | | | (Drosophila) |
| 225326_at | 2.82E−05 | 0.7 | RBM27 | RNA binding motif protein 27 |
| 229903_x_at | 2.82E−05 | 0.6 | RNP | U11/U12 snRNP 65K |
| 203818_s_at | 8.96E−06 | 0.7 | SF3A3 | splicing factor 3a, subunit 3, 60 kDa |
| 214305_s_at | 0.00806 | 0.4 | SF3B1 | splicing factor 3b, subunit 1, 155 kDa |
| 203380_x_at | 0.000339 | 0.7 | SFRS5 | splicing factor, arginine/serine-rich 5 |
| 217833_at | 2.82E−05 | 0.7 | SYNCRIP | Synaptotagmin binding, cytoplasmic RNA |
| | | | | interacting protein |

intracellular signaling cascade

| | | | | |
|---|---|---|---|---|
| 209409_at | 0.00392 | 2.1 | GRB10 | growth factor receptor-bound protein 10 |
| 212873_at | 0.00566 | 0.7 | HA-1 | minor histocompatibility antigen HA-1 |
| 206302_s_at | 8.07E−05 | 6.6 | NUDT4 | nudix (nucleoside diphosphate linked moiety |
| | | | | X)-type motif 4 |
| 206303_s_at | 4.86E−06 | 7.2 | NUDT4 | nudix (nucleoside diphosphate linked moiety |
| | | | | X)-type motif 4 |
| 212239_at | 0.00123 | 0.5 | PIK3R1 | phosphoinositide-3-kinase, regulatory subunit |
| | | | | 1 (p85 alpha) |
| 229980_s_at | 0.000133 | 0.4 | SNX5 | sorting nexin 5 |
| 221748_s_at | 1.30E−06 | 12.4 | TNS | tensin |
| 226255_at | 0.000528 | 0.7 | ZBTB33 | zinc finger and BTB domain containing 33 |

RNA processing

| | | | | |
|---|---|---|---|---|
| 211623_s_at | 0.000214 | 0.6 | FBL | fibrillarin |
| 201054_at | 0.000339 | 0.7 | HNRPA0 | heterogeneous nuclear ribonucleoprotein A0 |
| 232004_at | 8.07E−05 | 0.6 | HNRPR | Heterogeneous nuclear ribonucleoprotein R |
| 201517_at | 4.82E−05 | 0.6 | NCBP2 | nuclear cap binding protein subunit 2, 20 kDa |
| 208319_s_at | 0.00806 | 0.5 | RBM3 | RNA binding motif (RNP1, RRM) protein 3 |
| 206111_at | 8.96E−06 | 4.0 | RNASE2 | ribonuclease, RNase A family, 2 (liver, |
| | | | | eosinophil-derived neurotoxin) |
| 228370_at | 2.82E−05 | 0.6 | SNRPN | SNRPN upstream reading frame |
| 201522_x_at | 0.000339 | 0.7 | SNRPN /// | small nuclear ribonucleoprotein polypeptide N |
| | | | SNURF | |
| 206042_x_at | 2.82E−05 | 0.7 | SNRPN /// | small nuclear ribonucleoprotein polypeptide N |
| | | | SNURF | |

ribosome biogenesis

| | | | | |
|---|---|---|---|---|
| 203082_at | 0.0027 | 0.7 | BMS1L | BMS1-like, ribosome assembly protein |
| | | | | (yeast) |
| 201948_at | 0.000133 | 0.6 | GNL2 | guanine nucleotide binding protein-like 2 |
| | | | | (nucleolar) |

Protein modification

| | | | | |
|---|---|---|---|---|
| 212406_s_at | 0.0027 | 0.7 | C20orf36 | chromosome 20 open reading frame 36 |
| 209391_at | 0.00123 | 2.0 | DPM2 | dolichyl-phosphate mannosyltransferase |
| | | | | polypeptide 2, regulatory subunit |
| 203367_at | 0.00803 | 0.7 | DUSP14 | dual specificity phosphatase 14 |
| 226119_at | 0.00806 | 0.6 | LOC115294 | similar to hypothetical protein FLJ10883 |
| 202197_at | 0.0027 | 1.7 | MTMR3 | myotubularin related protein 3 |
| 205005_s_at | 4.86E−06 | 0.5 | NMT2 | N-myristoyltransferase 2 |
| 203966_s_at | 0.00081 | 4.6 | PPM1A | protein phosphatase 1A (formerly 2C), |
| | | | | magnesium-dependent, alpha isoform /// |
| | | | | protein phosphatase 1A (formerly 2C), |
| | | | | magnesium-dependent, alpha isoform |
| 208615_s_at | 4.82E−05 | 0.5 | PTP4A2 | protein tyrosine phosphatase type IVA, |
| | | | | member 2 |
| 209180_at | 0.00393 | 0.6 | RABGGTB | Rab geranylgeranyltransferase, beta subunit |
| 217977_at | 0.000133 | 2.4 | SEPX1 | selenoprotein X, 1 |
| 222989_s_at | 0.00081 | 2.2 | UBQLN1 | ubiquilin 1 |

DNA

| | | | | |
|---|---|---|---|---|
| 212672_at | 0.000339 | 0.5 | ATM | Ataxia telangiectasia mutated (includes |
| | | | | complementation groups A, C and D) |
| 218877_s_at | 0.00123 | 0.5 | C6orf75 | chromosome 6 open reading frame 75 |
| 223518_at | 4.86E−06 | 1.9 | DFFA | DNA fragmentation factor, 45 kDa, alpha |
| | | | | polypeptide |
| 228131_at | 0.000339 | 0.6 | ERCC1 | Excision repair cross-complementing rodent |
| | | | | repair deficiency, complementation group 1 |
| | | | | (includes overlapping antisense sequence) |
| 202414_at | 0.00081 | 0.5 | ERCC5 | excision repair cross-complementing rodent |
| | | | | repair deficiency, complementation group 5 |
| | | | | (xeroderma pigmentosum, camplementation |
| | | | | group G (Cackayne syndrome)) |
| 200792_at | 8.07E−05 | 0.7 | G22P1 | thyroid autoantigen 70 kDa (Ku antigen) |
| 204528_s_at | 0.000528 | 0.6 | NAP1L1 | nucleosome assembly protein 1-like 1 |
| 212967_x_at | 8.07E−05 | 0.7 | NAP1L1 | nucleosome assembly protein 1-like 1 |
| 213864_s_at | 0.000528 | 0.6 | NAP1L1 | nucleosome assembly protein 1-like 1 |
| 203939_at | 0.00393 | 0.5 | NT5E | 5′-nucleotidase, ecto (CD73) |
| 212917_x_at | 0.000528 | 0.6 | RECQL | RecQ protein-like (DNA helicase Q1-like) |
| 213047_x_at | 0.00806 | 0.7 | SET | SET translocation (myeloid leukemia- |
| | | | | associated) |
| 40189_at | 0.00393 | 0.7 | SET | SET translocation (myeloid leukemia- |
| | | | | associated) |
| 208901_s_at | 0.00806 | 1.8 | TOP1 | topoisomerase (DNA) I |
| 201513_at | 0.000528 | 0.5 | TSN | translin |

nucleosome assembly

| | | | | |
|---|---|---|---|---|
| 208886_at | 0.00123 | 2.2 | H1F0 | H1 histone family, member 0 |
| 209398_at | 0.000528 | 4.1 | HIST1H1C | histone 1, H1c |
| 221493_at | 0.00566 | 0.7 | TSPYL1 | TSPY-like 1 |

cell
adhesion

| | | | | |
|---|---|---|---|---|
| 226016_at | 0.00183 | 0.6 | CD47 | CD47 antigen (Rh-related antigen, integrin- |
| | | | | associated signal transducer) |
| 202468_s_at | 1.30E−06 | 5.4 | CTNNAL1 | catenin (cadherin-associated protein), alpha- |
| | | | | like 1 |
| 226817_at | 8.07E−05 | 2.6 | DSC2 | desmocollin 2 |
| 204714_s_at | 0.000133 | 2.7 | F5 | coagulation factor V (proaccelerin, labile |
| | | | | factor) |
| 205786_s_at | 0.0027 | 1.6 | ITGAM | integrin, alpha M (complement component |
| | | | | receptor 3, alpha; also known as CD11b |
| | | | | (p170), macrophage antigen alpha |
| | | | | polypeptide) /// integrin, alpha M (complement |
| | | | | component receptor 3, alpha; also known as |
| | | | | CD11b (p170), macrophage antigen alpha |
| | | | | polypeptide) |
| 204563_at | 0.00123 | 1.4 | SELL | selectin L (lymphocyte adhesion molecule 1) |
| 225246_at | 0.000528 | 0.7 | STIM2 | stromal interaction molecule 2 |
| 215706_x_at | 0.000214 | 2.2 | ZYX | zyxin |

chromatin

| | | | | |
|---|---|---|---|---|
| 238043_at | 0.00081 | 0.6 | ARID1B | AT rich interactive domain 1B (SWI1-like) |
| 205062_x_at | 0.000339 | 0.5 | ARID4A | AT rich interactive domain 4A (RBP1-like) |
| 227558_at | 0.00393 | 0.5 | CBX4 | chromobox homolog 4 (Pc class homolog, |
| | | | | Drosophila) |
| 213251_at | 0.00393 | 0.6 | SMARCA5 | SWI/SNF related, matrix associated, actin |
| | | | | dependent regulator of chromatin, subfamily |
| | | | | a, member 5 |

other

| | | | | |
|---|---|---|---|---|
| 211560_s_at | 1.30E−06 | 593.4 | ALAS2 | aminolevulinate, delta-, synthase 2 |
| | | | | (sideroblastic/hypochromic anemia) |
| 201366_at | 8.96E−06 | 0.6 | ANXA7 | annexin A7 |
| 210027_s_at | 1.61E−05 | 0.7 | APEX1 | APEX nuclease (multifunctional DNA repair |
| | | | | enzyme) 1 |
| 244875_at | 1.30E−06 | 5.8 | ASMTL | Acetylserotonin O-methyltransferase-like |
| 208677_s_at | 2.56E−06 | 4.7 | BSG | basigin (OK blood group) |
| 211727_s_at | 0.00183 | 0.6 | COX11 | COX11 homolog, cytochrome c oxidase |
| | | | | assembly protein (yeast) /// COX11 homolog, |
| | | | | cytochrome c oxidase assembly protein |
| | | | | (yeast) |
| 215001_s_at | 2.82E−05 | 2.0 | GLUL | glutamate-ammonia ligase (glutamine |
| | | | | synthase) |
| 217202_s_at | 2.56E−06 | 5.4 | GLUL | glutamate-ammonia ligase (glutamine |
| | | | | synthase) |
| 220404_at | 8.90E−06 | 5.0 | GPR97 | G protein-coupled receptor 97 |
| 200075_s_at | 2.82E−05 | 2.8 | GUK1 | guanylate kinase 1 /// guanylate kinase 1 |
| 202947_s_at | 4.86E−06 | 5.4 | GYPC | glycophorin C (Gerbich blood group) |
| 214470_at | 0.00566 | 0.4 | KLRB1 | killer cell lectin-like receptor subfamily B, |
| | | | | member 1 /// killer cell lectin-like receptor |
| | | | | subfamily B, member 1 |
| 227250_at | 0.000339 | 7.4 | KREMEN1 | Kringle containing transmembrane protein 1 |
| 201153_s_at | 0.000133 | 0.7 | MBNL1 | muscleblind-like (Drosophila) |
| 203774_at | 0.00081 | 0.6 | MTR | 5-methyltetrahydrofolate-homocysteine |
| | | | | methyltransferase |
| 201707_at | 0.000339 | 0.6 | PEX19 | peroxisomal biogenesis factor 19 |
| 202446_s_at | 4.86E−06 | 3.1 | PLSCR1 | phospholipid scramblase 1 |
| 200845_s_at | 0.000214 | 0.6 | PRDX6 | peroxiredoxin 6 |
| 226577_at | 2.82E−05 | 1.5 | PSEN1 | Presenilin 1 (Alzheimer disease 3) |
| 218428_s_at | 1.61E−05 | 0.6 | REV1L | REV1-like (yeast) |
| 46665_at | 0.0027 | 0.5 | SEMA4C | sema domain, immunoglobulin domain (Ig), |
| | | | | transmembrane domain (TM) and short |
| | | | | cytoplasmic domain, (semaphorin) 4C |
| 200652_at | 2.82E−05 | 0.6 | SSR2 | signal sequence receptor, beta (translocon- |
| | | | | associated protein beta) |
| 203887_s_at | 0.000528 | 4.8 | THBD | thrombomodulin |
| 207196_s_at | 8.96E−06 | 1.8 | TNIP1 | TNFAIP3 interacting protein 1 |
| 205672_at | 0.000214 | 0.6 | XPA | xeroderma pigmentosum, complementation |
| | | | | group A |
| 227594_at | 0.000528 | 0.6 | ZNF258 | zinc finger protein 258 |

Unknown

| | | | | |
|---|---|---|---|---|
| 205566_at | 0.00123 | 1.8 | ABHD2 | abhydrolase domain containing 2 |
| 226665_at | 0.00081 | 0.4 | AHSA2 | AHA1, activator of heat shock 90 kDa protein |
| | | | | ATPase homolog 2 (yeast) |
| 212174_at | 8.03E−05 | 0.7 | AK2 | adenylate kinase 2 |
| 226718_at | 2.82E−05 | 0.5 | AMIGO | amphoterin-induced gene and ORF |
| 222108_at | 0.00081 | 0.6 | AMIGO2 | amphoterin induced gene 2 |
| 238439_at | 0.00183 | 6.1 | ANKRD22 | ankyrin repeat domain 22 |
| 239196_at | 0.000528 | 2.5 | ANKRD22 | ankyrin repeat domain 22 |
| 230972_at | 4.82E−05 | 6.7 | ANKRD9 | ankyrin repeat domain 9 |
| 209369_at | 0.000337 | 15.4 | ANXA3 | annexin A3 |
| 202492_at | 0.00081 | 2.0 | APG9L1 | APG9 autophagy 9-like 1 (S. cerevisiae) |
| 225618_at | 0.00183 | 0.6 | ARHGAP27 | Rho GTPase activating protein 27 |
| 202655_at | 2.82E−05 | 2.1 | ARMET | arginine-rich, mutated in early stage tumors |
| 226055_at | 1.30E−06 | 0.5 | ARRDC2* | arrestin domain containing 2 |
| 215440_s_at | 1.30E−06 | 0.5 | BEXL1 | brain expressed X-linked-like 1 |
| 202201_at | 4.82E−05 | 4.7 | BLVRB | biliverdin reductase B (flavin reductase |
| | | | | (NADPH)) |
| 209846_s_at | 0.000528 | 0.5 | BTN3A2 | butyrophilin, subfamily 3, memberA2 |
| 55662_at | 0.000339 | 0.7 | C10orf76 | chromosome 10 open reading frame 76 |
| 213239_at | 1.61E−05 | 0.5 | C13orf24 | chromosome 13 open reading frame 24 |
| 218572_at | 2.56E−06 | 0.4 | C14orf123 | chromosome 14 open reading frame 123 |
| 221932_s_at | 1.30E−06 | 10.7 | C14orf87* | chromosome 14 open reading frame 87 |
| 203289_s_at | 4.86E−06 | 9.2 | C16orf35 | chromosome 16 open reading frame 35 |
| 214273_x_at | 2.82E−05 | 5.6 | C16orf35 | chromosome 16 open reading frame 35 |
| 221764_at | 2.56E−06 | 4.0 | C19orf22 | chromosome 19 open reading frame 22 |
| 55705_at | 1.30E−06 | 2.9 | C19orf22* | chromosome 19 open reading frame 22 |
| 226105_at | 0.00183 | 0.6 | C1GALT1 | Core 1 UDP-galactose: N- |
| | | | | acetylgalactosamine-alpha-R beta 1,3- |
| | | | | galactosyltransferase |
| 224690_at | 8.96E−06 | 7.8 | C20orf108 | chromosome 20 open reading frame 108 |
| 224693_at | 1.30E−06 | 5.3 | C20orf108* | chromosome 20 open reading frame 108 |
| 225252_at | 0.000528 | 2.5 | C20orf139 | chromosome 20 open reading frame 139 |
| 228291_s_at | 0.00566 | 0.7 | C20orf19 | chromosome 20 open reading frame 19 |
| 223039_at | 0.00183 | 2.4 | C22orf13 | chromosome 22 open reading frame 13 |
| 218518_at | 4.82E−05 | 0.6 | C5orf5 | chromosome 5 open reading frame 5 |
| 220755_s_at | 2.56E−06 | 0.6 | C6orf48 | chromosome 6 open reading frame 48 |
| 226443_at | 2.82E−05 | 1.6 | C9orf42 | chromosome 9 open reading frame 42 |
| 218929_at | 0.00393 | 0.7 | CARF | collaborates/cooperates with ARF (alternate |
| | | | | reading frame) protein |
| 223084_s_at | 0.0027 | 2.4 | CCNDBP1 | cyclin D-type binding-protein 1 |
| 34210_at | 4.86E−06 | 0.6 | CD52 | CD52 antigen (CAMPATH-1 antigen) |
| 200663_at | 0.00123 | 1.9 | CD63 | CD63 antigen (melanoma 1 antigen) |
| 204577_s_at | 1.30E−06 | 0.4 | CLUAP1* | clusterin associated protein 1 |
| 223431_at | 0.0027 | 0.7 | CNO | cappuccino |
| 222702_x_at | 1.30E−06 | 1.6 | CRIPT* | postsynaptic protein CRIPT |
| 225216_at | 0.00183 | 0.6 | CXorf39 | chromosome X open reading frame 39 |
| 242292_at | 0.000133 | 0.5 | CXorf50 | chromosome X open reading frame 50 |
| 215785_s_at | 2.82E−05 | 0.5 | CYFIP2 | cytoplasmic FMR1 interacting protein 2 |
| 212690_at | 0.00081 | 0.6 | DDHD2 | DDHD domain containing 2 |
| 201788_at | 0.0027 | 0.6 | DDX42 | DEAD (Asp-Glu-Ala-Asp) box polypeptide 42 |
| 228039_at | 4.82E−05 | 0.5 | DDX46 | DEAD (Asp-Glu-Ala-Asp) box polypeptide 46 |
| 213701_at | 0.0027 | 0.5 | DKFZp434N2030 | hypothetical protein DKFZp434N2030 |
| 227309_at | 0.00183 | 5.3 | DKFZp451J1719 | hypothetical DKFZp451J1719 |
| 202537_s_at | 0.0027 | 0.6 | DKFZP564O123 | DKFZP564O123 protein |
| 226657_at | 1.61E−05 | 2.6 | DKFZp762H185 | hypothetical protein DKFZp762H185 |
| 225405_at | 0.000214 | 0.6 | DKFZp762N1910 | Hypothetical protein DKFZp762N1910 |
| 220320_at | 1.61E−05 | 2.4 | DOK3 | docking protein 3 |
| 223553_s_at | 0.000133 | 2.9 | DOK3 | docking protein 3 |
| 226009_at | 0.000337 | 2.0 | DPCD | deleted in a mouse model of primary ciliary |
| | | | | dyskinesia |
| 212830_at | 8.07E−05 | 1.9 | EGFL5 | EGF-like-domain, multiple 5 |
| 212653_s_at | 0.00393 | 0.6 | EHBP1 | EH domain binding protein 1 |
| 215096_s_at | 0.000339 | 0.7 | ESD | esterase D/formylglutathione hydrolase |
| 218100_s_at | 1.30E−06 | 0.5 | ESRRBL1* | estrogen-related receptor beta like 1 |
| 241981_at | 0.00183 | 5.0 | FAM20A | family with sequence similarity 20, member A |
| 220547_s_at | 0.00566 | 0.6 | FAM35A | family with sequence similarity 35, member A |
| 224820_at | 0.00123 | 0.7 | FAM36A | family with sequence similarity 36, member A |
| 201889_at | 0.000339 | 0.6 | FAM3C | family with sequence similarity 3, member C |
| 225030_at | 0.00123 | 0.7 | FAM44B | family with sequence similarity 44, member B |
| 226811_at | 1.30E−06 | 10.1 | FAM46C* | family with sequence similarity 46, member C |
| 204335_at | 0.00566 | 0.5 | FLJ10374 | hypothetical protein FLJ10374 |
| 218545_at | 8.07E−05 | 0.7 | FLJ11088 | GGA binding partner |
| 217828_at | 0.00566 | 0.7 | FLJ13213 | hypothetical protein FLJ13213 |
| 225350_s_at | 0.00566 | 0.8 | FLJ13456 | Hypothetical protein FLJ13456 |
| 226521_s_at | 0.00123 | 0.6 | FLJ13614 | hypothetical protein FLJ13614 |
| 233543_s_at | 0.00803 | 0.7 | FLJ13614 | hypothetical protein FLJ13614 |
| 212995_x_at | 1.61E−05 | 0.6 | FLJ14346 | hypothetical protein FLJ14346 |
| 225319_s_at | 0.0027 | 2.6 | FLJ14775 | hypothetical protein FLJ14775 |
| 218532_s_at | 0.00393 | 0.5 | FLJ20152 | hypothetical protein FLJ20152 |
| 219093_at | 0.000339 | 0.4 | FLJ20701 | hypothetical protein FLJ20701 /// hypothetical |
| | | | | protein FLJ20701 |
| 218932_at | 0.00123 | 0.6 | FLJ20729 | hypothetical protein FLJ20729 |
| 51200_at | 0.00123 | 0.5 | FLJ20850 | hypothetical protein FLJ20850 |
| 223528_s_at | 0.00183 | 0.6 | FLJ20859 | FLJ20859 gene |
| 219029_at | 0.000528 | 0.5 | FLJ21657 | hypothetical protein FLJ21657 |
| 218842_at | 0.00183 | 0.7 | FLJ21908 | hypothetical protein FLJ21908 |
| 218454_at | 0.000214 | 2.3 | FLJ22662 | hypothetical protein FLJ22662 |
| 235052_at | 0.000214 | 0.4 | FLJ38451 | FLJ38451 protein |
| 64432_at | 2.82E−05 | 0.5 | FLJ39616 | apoptosis-related protein PNAS-1 |
| 208749_x_at | 4.82E−05 | 2.1 | FLOT1 | flotillin 1 |
| 210142_x_at | 8.96E−06 | 2.5 | FLOT1 | flotillin 1 |
| 202232_s_at | 4.82E−05 | 0.5 | GA17 | dendritic cell protein |
| 224719_s_at | 0.00081 | 0.6 | GRCC10 | likely ortholog of mouse gene rich cluster, |
| | | | | C10 gene |
| 211820_x_at | 2.56E−06 | 8.3 | GYPA | glycophorin A (includes MN blood group) |
| 211821_x_at | 1.30E−06 | 36.0 | GYPA* | glycophorin A (includes MN blood group) |
| 207459_x_at | 8.96E−06 | 9.1 | GYPB | glycophorin B (includes Ss blood group) |
| 214407_x_at | 1.30E−06 | 10.5 | GYPB | glycophorin B (includes Ss blood group) |
| 216833_x_at | 0.000133 | 5.8 | GYPB | glycophorin B (includes Ss blood group) /// |
| | | | | glycophorin E |
| 205012_s_at | 2.56E−06 | 2.9 | HAGH | hydroxyacylglutathione hydrolase |
| 217414_x_at | 0.00806 | 3.1 | HBA2 | hemoglobin, alpha 2 |
| 221425_s_at | 0.00393 | 3.8 | HBLD2 | HESB like domain containing 2 /// HESB like |
| | | | | domain containing 2 |
| 225584_at | 0.00081 | 0.5 | HCG18 | CDNA clone IMAGE: 5265581, partial cds |
| 217965_s_at | 1.61E−05 | 0.5 | HCNGP | transcriptional regulator protein |
| 223252_at | 0.00123 | 1.6 | HDGF2 | hepatoma-derived growth factor-related |
| | | | | protein 2 |
| 228736_at | 0.00806 | 0.6 | HEL308 | DNA helicase HEL308 |
| 223670_s_at | 2.82E−05 | 10.2 | HEMGN | hemogen |
| 218946_at | 0.00081 | 0.6 | HIRIP5 | HIRA interacting protein 5 |
| 214290_s_at | 0.00123 | 2.4 | HIST2H2AA | histone 2, H2aa |
| 218280_x_at | 0.000528 | 2.1 | HIST2H2AA | histone 2, H2aa |
| 232209_x_at | 0.00566 | 0.5 | HM13 | histocompatibility (minor) 13 |
| 209787_s_at | 0.0027 | 0.7 | HMGN4 | high mobility group nucleosomal binding |
| | | | | domain 4 |
| 211929_at | 8.96E−06 | 0.5 | HNRPA3 | heterogeneous nuclear ribonucleoprotein A3 |
| 203203_s_at | 0.00393 | 0.5 | HRB2 | HIV-1 rev binding protein 2 |
| 225845_at | 0.000339 | 0.6 | HSPC063 | HSPC063 protein |
| 223124_s_at | 0.000133 | 5.6 | HT014 | HT014 |
| 213804_at | 0.00183 | 0.5 | INPP5B | inositol polyphosphate-5-phosphatase, 75 kDa |
| 218569_s_at | 0.00081 | 0.4 | KBTBD4 | kelch repeat and BTB (POZ) domain |
| | | | | containing 4 |
| 212267_at | 0.00081 | 0.7 | KIAA0261 | KIAA0261 |
| 212355_at | 0.0027 | 1.4 | KIAA0323 | KIAA0323 |
| 204308_s_at | 0.000528 | 1.7 | KIAA0329 | KIAA0329 |
| 201855_s_at | 0.00183 | 0.6 | KIAA0431 | KIAA0431 protein |
| 212675_s_at | 0.00566 | 0.5 | KIAA0582 | KIAA0582 |
| 34260_at | 0.00564 | 0.5 | KIAA0683 | KIAA0683 gene product |
| 212201_at | 0.00806 | 0.7 | KIAA0692 | KIAA0692 protein |
| 228549_at | 4.86E−06 | 0.5 | KIAA0792 | KIAA0792 gene product |
| 230546_at | 0.00806 | 1.6 | KIAA1036 | KIAA1036 |
| 212754_s_at | 0.000339 | 0.6 | KIAA1040 | KIAA1040 protein |
| 221495_s_at | 0.00393 | 0.7 | KIAA1049 | KIAA1049 protein |
| 207765_s_at | 0.00806 | 2.2 | KIAA1539 | KIAA1539 |
| 211433_x_at | 0.0027 | 2.4 | KIAA1539 | KIAA1539 |
| 231850_x_at | 1.30E−06 | 0.5 | KIAA1712 | KIAA1712 |
| 234671_at | 0.000339 | 2.1 | KRTAP4-2 | keratin associated protein 4-2 |
| 208117_s_at | 0.000133 | 0.6 | LAS1L | LAS1-like (S. cerevisiae) |
| 223162_s_at | 8.96E−06 | 0.6 | LCHN | LCHN protein |
| 209179_s_at | 0.000133 | 2.8 | LENG4 | leukocyte receptor cluster (LRC) member 4 |
| 203276_at | 0.00566 | 3.1 | LMNB1 | lamin B1 |
| 228930_at | 2.56E−06 | 0.5 | LOC123722 | Hypothetical protein LOC123722 |
| 235568_at | 8.07E−05 | 6.3 | LOC199675 | hypothetical protein LOC199675 |
| 235587_at | 1.30E−06 | 0.5 | LOC202781* | hypothetical protein LOC202781 |
| 35156_at | 0.00081 | 0.6 | LOC203069 | hypothetical protein LOC203069 |
| 222662_at | 8.07E−05 | 2.4 | LOC286044 | hypothetical protein LOC286044 |
| 229323_at | 0.00123 | 0.4 | LOC387723 | hypothetical LOC387723 |
| 216565_x_at | 0.00806 | 1.9 | LOC391020 | similar to Interferon-induced transmembrane |
| | | | | protein 3 (Interferon-inducible protein 1-8 U) |
| 225635_s_at | 0.000214 | 0.5 | LOC401504 | hypothetical gene supported by AK091718 |
| 240890_at | 4.82E−05 | 0.5 | LOC440066 | similar to Caspase-4 precursor (CASP-4) |
| | | | | (ICH-2 protease) (TX protease) (ICE(rel)-II) |
| 229872_s_at | 0.000339 | 0.6 | LOC440667 | LOC440667 |
| 237563_s_at | 0.000214 | 2.2 | LOC440731 | LOC440731 |
| 226686_at | 0.00081 | 3.3 | LOC493856 | similar to RIKEN cDNA 1500009M05 gene |
| 226689_at | 0.00393 | 3.4 | LOC493856 | similar to RIKEN cDNA 1500009M05 gene |
| 217882_at | 2.82E−05 | 2.2 | LOC55831 | 30 kDa protein |
| 228775_at | 0.00081 | 2.1 | LOC55831 | 30 kDa protein |
| 225705_at | 0.00566 | 0.6 | LOC90799 | hypothetical protein BC009518 |
| 235778_s_at | 0.00183 | 0.5 | LOC91526 | hypothetical protein DKFZp434D2328 |
| 213224_s_at | 0.00806 | 0.5 | LOC92482 | hypothetical protein LOC92482 |
| 228993_s_at | 8.07E−05 | 0.4 | LOC92482 | hypothetical protein LOC92482 |
| 210102_at | 1.61E−05 | 2.1 | LOH11CR2A | loss of heterozygosity, 11, chromosomal |
| | | | | region 2, gene A |
| 228253_at | 2.82E−05 | 1.5 | LOXL3 | lysyl oxidase-like 3 |
| 219630_at | 0.00566 | 3.9 | MAP17 | membrane-associated protein 17 |
| 220603_s_at | 0.0027 | 2.4 | MCTP2 | multiple C2-domains with two transmembrane |
| | | | | regions 2 |
| 223754_at | 0.000214 | 3.3 | MGC13057 | hypothetical protein MGC13057 |
| 227402_s_at | 8.07E−05 | 0.4 | MGC14595 | hypothetical protein MGC14595 |
| 226448_at | 8.96E−06 | 2.4 | MGC15887 | hypothetical gene supported by BC009447 |
| 224759_s_at | 0.00566 | 0.5 | MGC17943 | hypothetical protein MGC17943 |
| 212340_at | 0.000528 | 2.4 | MGC21416 | hypothetical protein MGC21416 |
| 204985_s_at | 0.00566 | 0.5 | MGC2650 | hypothetical protein MGC2650 |
| 229736_at | 0.0027 | 2.1 | MGC30208 | hypothetical protein MGC30208 |
| 235005_at | 0.00183 | 0.5 | MGC4562 | hypothetical protein MGC4562 |
| 221580_s_at | 0.00393 | 0.7 | MGC5306 | hypothetical protein MGC5306 |
| 220615_s_at | 0.00393 | 3.2 | MLSTD1 | male sterility domain containing 1 |
| 239108_at | 0.00806 | 2.4 | MLSTD1 | Male sterility domain containing 1 |
| 211685_s_at | 0.000528 | 0.6 | NCALD | neurocalcin delta |
| 210097_s_at | 0.00183 | 0.7 | NOL7 | nucleolar protein 7, 27 kDa |
| 209007_s_at | 0.000214 | 0.4 | NPD014 | NPD014 protein |
| 219458_s_at | 0.00123 | 1.9 | NSUN3 | NOL1/NOP2/Sun domain family, member 3 |
| 203718_at | 0.000133 | 2.0 | NTE | neuropathy target esterase |
| 200649_at | 8.07E−05 | 2.0 | NUCB1 | nucleobindin 1 |
| 217802_s_at | 0.00393 | 0.6 | NUCKS | nuclear ubiquitous casein kinase and cyclin- |
| | | | | dependent kinase substrate |
| 226726_at | 2.56E−06 | 4.0 | OACT2 | O-acyltransferase (membrane bound) domain |
| | | | | containing 2 |
| 223011_s_at | 8.96E−06 | 0.6 | OCIAD1 | OCIA domain containing 1 |
| 241881_at | 2.56E−06 | 13.4 | OR2W3 | olfactory receptor, family 2, subfamily W, |
| | | | | member 3 |
| 201245_s_at | 0.00393 | 0.8 | OTUB1 | OTU domain, ubiquitin aldehyde binding 1 |
| 202671_s_at | 0.00183 | 1.7 | PDXK | pyridoxal (pyridoxine, vitamin B6) kinase |
| 33760_at | 0.000528 | 0.6 | PEX14 | peroxisomal biogenesis factor 14 |
| 201701_s_at | 0.00081 | 0.5 | PGRMC2 | progesterone receptor membrane component 2 |
| 226247_at | 2.82E−05 | 0.5 | PLEKHA1 | pleckstrin homology domain containing, |
| | | | | family A (phosphoinositide binding specific) |
| | | | | member 1 |
| 212705_x_at | 8.07E−05 | 2.7 | PNPLA2 | patatin-like phospholipase domain containing 2 |
| 207000_s_at | 2.82E−05 | 0.5 | PPP3CC | protein phosphatase 3 (formerly 2B), catalytic |
| | | | | subunit, gamma isoform (calcineurin A |
| | | | | gamma) |
| 32541_at | 8.07E−05 | 0.5 | PPP3CC | protein phosphatase 3 (formerly 2B), catalytic |
| | | | | subunit, gamma isoform (calcineurin A |
| | | | | gamma) |
| 204507_s_at | 8.96E−06 | 3.3 | PPP3R1 | protein phosphatase 3 (formerly 2B), |
| | | | | regulatory subunit B, 19 kDa, alpha isoform |
| | | | | (calcineurin B, type I) |
| 209337_at | 0.000339 | 0.6 | PSIP1 | PC4 and SFRS1 interacting protein 1 |
| 202897_at | 0.000337 | 2.1 | PTPNS1 | protein tyrosine phosphatase, non-receptor |
| | | | | type substrate 1 |
| 212168_at | 0.00393 | 0.7 | RBM12 | RNA binding motif protein 12 |
| 225310_at | 4.82E−05 | 0.6 | RBMX | RNA binding motif protein, X-linked |
| 213338_at | 0.0027 | 10.5 | RIS1 | Ras-induced senescence 1 |
| 223609_at | 0.000214 | 2.5 | ROPN1L | ropporin 1-like |
| 223656_s_at | 0.00081 | 1.5 | RP4-622L5 | hypothetical protein RP4-622L5 |
| 205087_at | 8.07E−05 | 0.6 | RWDD3 | RWD domain containing 3 |
| 214433_s_at | 1.30E−06 | 101.4 | SELENBP1 | selenium binding protein 1 |
| 233587_s_at | 0.00081 | 2.2 | SIPA1L2 | signal-induced proliferation-associated 1 like 2 |
| 203021_at | 0.00123 | 4.4 | SLPI | secretory leukocyte protease inhibitor |
| | | | | (antileukoproteinase) |
| 211988_at | 4.86E−06 | 0.5 | SMARCE1 | SWI/SNF related, matrix associated, actin |
| | | | | dependent regulator of chromatin, subfamily |
| | | | | e, member 1 |
| 224640_at | 0.000528 | 1.7 | SPPL3 | signal peptide peptidase 3 |
| 201225_s_at | 0.000994 | 0.8 | SRRM1 | serine/arginine repetitive matrix 1 |
| 207320_x_at | 0.00081 | 1.7 | STAU | staufen, RNA binding protein (Drosophila) |
| 208948_s_at | 0.00566 | 1.4 | STAU | staufen, RNA binding protein (Drosophila) |
| 225396_at | 1.30E−06 | 0.6 | SYNCOILIN* | Intermediate filament protein syncoilin |
| 223231_at | 0.00081 | 0.6 | TATDN1 | TatD DNase domain containing 1 |
| 226664_at | 0.000214 | 2.0 | TBC1D20 | TBC1 domain family, member 20 |
| 208089_s_at | 0.00269 | 0.5 | TDRD3 | tudor domain containing 3 /// tudor domain |
| | | | | containing 3 |
| 206555_s_at | 0.000132 | 0.5 | THUMPD1 | THUMP domain containing 1 |
| 217979_at | 4.82E−05 | 0.4 | TM4SF13 | transmembrane 4 superfamily member 13 |
| 209890_at | 8.07E−05 | 7.4 | TM4SF9 | transmembrane 4 superfamily member 9 /// |
| | | | | transmembrane 4 superfamily member 9 |
| 225387_at | 0.000339 | 8.2 | TM4SF9 | transmembrane 4 superfamily member 9 |
| 225388_at | 8.96E−06 | 4.3 | TM4SF9 | transmembrane 4 superfamily member 9 |
| 218872_at | 0.00806 | 2.7 | TSC | hypothetical protein FLJ20607 |
| 225180_at | 0.0027 | 0.6 | TTC14 | tetratricopeptide repeat domain 14 |
| 219192_at | 0.00564 | 0.7 | UBAP2 | ubiquitin associated protein 2 |
| 220757_s_at | 1.61E−05 | 5.2 | UBXD1 | UBX domain containing 1 |
| 223012_at | 0.000214 | 4.4 | UBXD1 | UBX domain containing 1 |
| 218050_at | 0.00183 | 0.5 | Ufm1 | ubiquitin-fold modifier 1 |
| 207628_s_at | 0.00123 | 0.7 | WBSCR22 | Williams Beuren syndrome chromosome |
| | | | | region 22 |
| 212602_at | 0.000133 | 2.8 | WDFY3 | WD repeat and FYVE domain containing 3 |
| 222804_x_at | 2.82E−05 | 1.9 | WDR32 | WD repeat domain 32 |
| 224789_at | 2.56E−06 | 9.0 | WDR40A | WD repeat domain 40A |
| 209216_at | 0.00393 | 2.2 | WDR45 | WD repeat domain 45 |
| 209217_s_at | 0.0027 | 1.7 | WDR45 | WD repeat domain 45 |
| 40829_at | 0.00123 | 1.6 | WDTC1 | WD and tetratricopeptide repeats 1 |
| 223179_at | 8.07E−05 | 3.3 | YPEL3 | yippee-like 3 (Drosophila) |
| 225629_s_at | 4.82E−05 | 0.7 | ZBTB4 | zinc finger and BTB domain containing 4 |
| 226496_at | 4.86E−06 | 0.5 | ZCCHC7 | zinc finger, CCHC domain containing 7 |
| 222730_s_at | 1.30E−06 | 3.0 | ZDHHC2* | zinc finger, DHHC domain containing 2 |
| 224593_at | 0.00123 | 0.6 | ZFOC1 | zinc finger protein ZFOC1 |
| 221848_at | 0.00393 | 0.6 | ZGPAT | zinc finger, CCCH-type with G patch domain |
| 57539_at | 0.00123 | 0.7 | ZGPAT | zinc finger, CCCH-type with G patch domain |
| 221626_at | 0.000339 | 0.5 | ZNF506 | zinc finger protein 506 |
| 227670_at | 2.82E−05 | 0.6 | ZNF75A | zinc finger protein 75a |
| 226680_at | 0.000528 | 0.6 | ZNFN1A5 | Zinc finger protein, subfamily 1A, 5 |
| 225131_at | 0.0027 | 2.2 | ZRANB1 | zinc finger, RAN-binding domain containing 1 |
| 212893_at | 0.00393 | 0.6 | ZZZ3 | zinc finger, ZZ domain containing 3 |
| 200067_x_at | 0.00806 | 2.0 | — | — |
| 208540_x_at | 0.000528 | 2.3 | — | — |
| 209193_at | 0.00806 | 2.5 | — | — |
| 211781_x_at | 4.82E−05 | 5.5 | — | — |
| 211994_at | 4.82E−05 | 2.8 | — | Clone A9A2BRB5 (CAC)n/(GTG)n repeat- |
| | | | | containing mRNA. /// Clone A9A2BRB5 |
| | | | | (CAC)n/(GTG)n repeat-containing mRNA. |
| 213048_s_at | 0.000528 | 0.5 | — | — |
| 213416_at | 0.00081 | 0.6 | — | — |
| 213608_s_at | 4.82E−05 | 4.7 | — | Similar to SRR1-like protein |
| 214394_x_at | 2.56E−06 | 0.5 | — | — |
| 215604_x_at | 4.82E−05 | 0.4 | — | — |
| 215963_x_at | 1.61E−05 | 0.5 | — | — |
| 216177_at | 0.000528 | 0.4 | — | — |
| 216508_x_at | 0.00392 | 0.6 | — | — |
| 216570_x_at | 1.30E−06 | 0.6 | — | — |
| 217019_at | 8.07E−05 | 0.4 | — | — |
| 217499_x_at | 0.000528 | 1.8 | — | PREDICTED: Homo sapiens olfactory |
| | | | | receptor, family 7, subfamily E, member 31 |
| | | | | pseudogene (OR7E31P), mRNA |
| 217946_s_at | 0.00566 | 0.7 | — | — |
| 221963_x_at | 0.000213 | 0.7 | — | — |
| 222431_at | 0.000339 | 0.6 | — | — |
| 224709_s_at | 0.00123 | 0.6 | — | — |
| 224752_at | 0.000528 | 3.0 | — | Homo sapiens, Similar to hypothetical protein |
| | | | | MGC10526, clone IMAGE: 4133906, mRNA |
| 224929_at | 0.00123 | 1.6 | — | — |
| 225176_at | 0.000528 | 0.6 | — | MSTP146 (MSTP146) |
| 225492_at | 0.00566 | 1.7 | — | CDNA FLJ32412 fis, clone SKMUS2000690 |
| 225595_at | 0.00806 | 0.3 | — | MRNA; cDNA DKFZp566P1124 (from clone |
| | | | | DKFZp566P1124) |
| 225856_at | 0.000339 | 0.6 | — | Homo sapiens, clone IMAGE: 5267398, |
| | | | | mRNA |
| 226179_at | 1.30E−06 | 20.9 | —* | FP15737 |
| 226272_at | 0.000528 | 0.6 | — | Full length insert cDNA clone ZD79H10 |
| 226542_at | 0.00566 | 1.9 | — | Full-length cDNA clone CS0DJ002YF02 of T |
| | | | | cells (Jurkat cell line) Cot 10-normalized of |
| | | | | Homo sapiens (human) |
| 226765_at | 0.00393 | 0.4 | — | — |
| 227184_at | 0.00123 | 2.0 | — | Transcribed locus, weakly similar to |
| | | | | NP_071385.1 chromosome 6 open reading |
| | | | | frame 79 [Homo sapiens] |
| 228390_at | 0.00183 | 0.4 | — | Homo sapiens, clone IMAGE: 5259272, |
| | | | | mRNA |
| 228634_s_at | 1.61E−05 | 3.0 | — | — |
| 228812_at | 0.00566 | 0.5 | — | Transcribed locus, weakly similar to |
| | | | | NP_689672.2 hypothetical protein |
| | | | | MGC45438 [Homo sapiens] |
| 228853_at | 0.00393 | 0.6 | — | — |
| 229064_s_at | 0.0027 | 0.5 | — | — |
| 229111_at | 0.00806 | 0.5 | — | — |
| 229220_x_at | 0.0027 | 0.4 | — | Homo sapiens, clone IMAGE: 4151011, |
| | | | | mRNA |
| 229373_at | 0.00393 | 2.3 | — | Transcribed locus |
| 229498_at | 0.00183 | 3.1 | — | MRNA; cDNA DKFZp779M2422 (from clone |
| | | | | DKFZp779M2422) |
| 229832_x_at | 4.82E−05 | 2.6 | — | — |
| 230208_at | 0.000528 | 2.1 | — | Transcribed locus |
| 230739_at | 0.00183 | 0.7 | — | CAMP-binding guanine nucleotide exchange |
| | | | | factor IV (cAMP-GEFIV) mRNA, clone W15, |
| | | | | partial sequence |
| 231039_at | 0.0027 | 0.6 | — | Transcribed locus |
| 231225_at | 0.00564 | 0.6 | — | Transcribed locus, weakly similar to |
| | | | | XP_512872.1 similar to Zinc finger protein 83 |
| | | | | (HPF1) [Pan troglodytes] |
| 231274_s_at | 8.96E−06 | 15.7 | — | — |
| 231688_at | 0.00806 | 30.3 | — | Transcribed locus |
| 233068_at | 0.000528 | 0.6 | — | CDNA FLJ13202 fis, clone NT2RP3004503 |
| 234969_s_at | 8.96E−06 | 0.6 | — | — |
| 234973_at | 0.000214 | 2.5 | — | — |
| 235014_at | 0.00081 | 0.6 | — | — |
| 235124_at | 0.00393 | 0.7 | — | CDNA FLJ35228 fis, clone PROST2001283 |
| 235199_at | 0.00566 | 0.6 | — | Transcribed locus, weakly similar to |
| | | | | XP_513408.1 similar to origin recognition |
| | | | | complex, subunit 1; origin recognition |
| | | | | complex, subunit 1, S. cerevisiae, homolog- |
| | | | | like; origin recognition complex 1; replication |
| | | | | control protein 1; origin recognition complex, |
| | | | | subunit 1 (yeast homolog)-like . . . [Pan |
| | | | | troglodytes] |
| 235466_s_at | 0.00806 | 0.5 | — | Transcribed locus |
| 236081_at | 1.30E−06 | 18.0 | — | Transcribed locus |
| 236196_at | 0.00806 | 0.7 | — | CDNA FLJ42548 fis, clone BRACE3004996 |
| 236198_at | 4.86E−06 | 0.4 | — | Transcribed locus |
| 236280_at | 0.00123 | 0.5 | — | Transcribed locus |
| 236301_at | 0.00123 | 0.5 | — | Full length insert cDNA clone YY82H04 |
| 237299_at | 0.000214 | 3.3 | — | Transcribed locus, moderately similar to |
| | | | | NP_055301.1 neuronal thread protein AD7c- |
| | | | | NTP [Homo sapiens] |
| 238431_at | 0.000339 | 0.6 | — | Transcribed locus, weakly similar to |
| | | | | NP_055301.1 neuronal thread protein AD7c- |
| | | | | NTP [Homo sapiens] |
| 239278_at | 0.000214 | 0.6 | — | Homo sapiens, clone IMAGE: 5301129, |
| | | | | mRNA |
| 241143_at | 0.00806 | 1.5 | — | — |
| 242104_at | 0.00806 | 0.5 | — | CDNA FLJ46553 fis, clone THYMU3038879 |
| 242335_at | 1.30E−06 | 6.4 | — | FP15737 |
| 242841_at | 0.00325 | 1.7 | — | Full length insert cDNA clone YS02G11 |
| 243024_at | 0.00393 | 1.5 | — | — |
| 244008_at | 2.82E−05 | 0.7 | — | Transcribed locus |
| 244189_at | 4.79E−05 | 0.5 | — | — |
| 36553_at | 1.30E−06 | 0.4 | —* | — |
| 65472_at | 0.00393 | 0.5 | — | Hypothetical LOC388969 |

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the. scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Cassidy J T, Petty, R. E. Textbook of Pediatric Rheumatology. 4th ed. Philadelphia: W. B. Saunders; 2001.
2. Wallace C A, Levinson J E. Juvenile rheumatoid arthritis: outcome and treatment for the 1990s. Rheum Dis Clin North Am 1991; 17(4):891-905.
3. Ravelli A, Martini A. Early predictors of outcome in juvenile idiopathic arthritis Clin Exp Rheumatol 2003; 21(5 Suppl 31):S89-93.
4. Modesto C, Woo P, Garcia-Consuegra J, et al. Systemic onset juvenile chronic arthritis, polyarticular pattern and hip involvement as markers for a bad prognosis. Clin Exp Rheumatol 2001; 19(2):211-7.
5. Ravelli A. Toward an understanding of the long-term outcome of juvenile idiopathic arthritis. Clin Exp Rheumatol 2004; 22(3):271-5.
6. Spiegel L R, Schneider R, Lang B A, et al. Early predictors of poor functional outcome in systemic-onset juvenile rheumatoid arthritis: a multicenter cohort study. Arthritis Rheum 2000; 43(11):2402-9.
7. Lomater C, Gerloni V, Gattinara M, Mazzotti J, Cimaz R, Fantini F. Systemic onset juvenile idiopathic arthritis: a retrospective study of 80 consecutive patients followed for 10 years. J Rheumatol 2000; 27(2):491-6.
8. Bowyer S L, Roettcher P A, Higgins G C, et al. Health status of patients with juvenile rheumatoid arthritis at 1 and 5 years after diagnosis. J Rheumatol 2003; 30(2):394-400.
9. Pascual V, Allantaz F, Arce E, Punaro M, Banchereau J. Role of interleukin-1 (IL-1) in the pathogenesis of systemic onset juvenile idiopathic arthritis and clinical response to IL-1 blockade. J Exp Med 2005; 201(9):1479-86.
10. Vasques Godinho F M, Parreira Santos M J, Canas da Silva J. Refractory adult onset Still's disease successfully treated with anakinra. Ann Rheum Dis 2005; 64(4):647-8.
11. Fitzgerald A A, Leclercq S A, Yan A, Homik J E, Dinarello C A. Rapid responses to anakinra in patients with refractory adult-onset Still's disease. Arthritis Rheum 2005; 52(6): 1794-803.
12. Zheng W, Flavell R A. The transcription factor GATA-3 is necessary and sufficient for Th2 cytokine gene expression in CD4 T cells. Cell 1997; 89(4):587-96.
13. Jonsson H, Allen P, Peng S L. Inflammatory arthritis requires Foxo3a to prevent Fas ligand-induced neutrophil apoptosis. Nat Med 2005; 11(6):666-71.
14. Kim J H, Park S M, Kang M R, et al. Ubiquitin ligase MKRN1 modulates telomere length homeostasis through a proteolysis of hTERT. Genes Dev 2005; 19(7):776-81.
15. Whitney A R, Diehn M, Popper S J, et al. Individuality and variation in gene expression patterns in human blood. Proc Natl Acad Sci USA 2003; 100(4):1896-901.

What is claimed is:

1. A method of identifying a human subject predisposed to systemic onset juvenile idiopathic arthritis comprising determining the expression level of a biomarker comprising one or more of the following genes: delta hemoglobin; erythroid associated factor; Kruppel-like factor 1; myosin light polypeptide 4; and makorin 1; wherein the biomarker is correlated with a predisposition to systemic onset juvenile idiopathic arthritis.

2. The method of claim 1, wherein the biomarker further comprises transcriptional regulation genes selected from upregulation of Foxo3a, downregulation of GATA-3 and combinations thereof.

3. The method of claim 1, wherein the biomarker further comprises inflammatory/immune response genes selected from upregulation IL-1 receptor antagonist (IL-1RN), downregulation Fc Epsilon receptor and combinations thereof.

4. The method of claim 1, wherein further comprises one or more biomarker selected from the following:

| | | |
|---|---|---|
| 213415_at | CLIC2* | chloride intracellular channel 2 |
| 225352_at | TLOC1* | translocation protein 1 |
| 225394_s_at | MADP-1* | MADP-1 protein |
| 211994_at | —* | Clone A9A2BRB5 (CAC)n/(GTG)n |
| | | repeat-containing mRNA |
| 212055_at | C18orf10* | chromosome 18 open reading frame 10 |
| 212174_at | AK2* | adenylate kinase 2 |
| | | similar to junction-mediating and |
| | | regulatory protein p300 |
| 228953_at | KIAA1971* | JMY |
| 230546_at | KIAA1036* | KIAA1036 |
| 230747_s_at | —* | CDNA clone IMAGE: 3029742, partial |
| | | cds |
| 242300_at | —* | — |
| 228622_s_at | DNAJC4* | DnaJ (Hsp40) homolog, subfamily C, |
| | | member 4 |
| 226296_s_at | MRPS15* | mitochondrial ribosomal protein S15 |

5. The method of claim 1, wherein the biomarker further comprises genes related to ubiquitination (solute carrier family 6/SLC6A8); components of the erythrocyte cytoskeleton (EBP42, tropomodulin 1); apoptosis (synuclein alpha) and combinations thereof.

6. The method of claim 1, wherein the screening is accomplished by quantitating the MRNA, protein or both mRNA and protein level of the biomarker.

7. The method of claim 1, wherein the biomarker comprises mRNA level and is quantitated by a method selected from the group consisting of polymerase chain reaction, real time polymerase chain reaction, reverse transcriptase polymerase chain reaction, hybridization, probe hybridization, and gene expression array.

8. The method of claim 1, wherein the screening further comprises detection of a polymorphism in the biomarker.

9. The method of claim 1, wherein the screening is accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing.

10. The method of claim 1, wherein the sample comprises a leukocyte.

11. A computer implemented method for determining the genotype of a sample comprising:
obtaining a plurality of sample probe intensities;
diagnosing systemic onset juvenile idiopathic arthritis based upon the sample probe intensities; and
calculating linear correlation coefficient between the sample probe intensities and reference probe intensities; and accepting the tentative genotype as the genotype of the sample if the linear correlation coefficient is greater than a threshold value.

12. The method of claim 11, wherein the threshold value is at least 0.8.

13. The method of claim 11, wherein the threshold value is at least 0.9.

14. The method of claim 11, wherein the threshold value is at least 0.95.

15. The method of claim 11, wherein the probe intensities are selected from a gene expression profile from the tissue sample wherein expression of the two or more of the following genes is measured:

| | | |
|---|---|---|
| 213415_at | CLIC2* | chloride intracellular channel 2 |
| 225352_at | TLOC1* | translocation protein 1 |
| 225394_s_at | MADP-1* | MADP-1 protein |
| 211994_at | —* | Clone A9A2BRB5 (CAC)n/(GTG)n |
| | | repeat-containing mRNA |
| 212055_at | C18orf10* | chromosome 18 open reading frame 10 |
| 212174_at | AK2* | adenylate kinase 2 |
| | | similar to junction-mediating and |
| | | regulatory protein p300 |
| 228953_at | KIAA1971* | JMY |
| 230546_at | KIAA1036* | KIAA1036 |
| 230747_s_at | —* | CDNA clone IMAGE: 3029742, partial |
| | | cds |
| 242300_at | —* | — |
| 228622_s_at | DNAJC4* | DnaJ (Hsp40) homolog, subfamily C, |
| | | member 4 |
| 226296_s_at | MRPS15* | mitochondrial ribosomal protein S15 |

as compared to a normal control sample.

16. A method for diagnosing systemic onset juvenile idiopathic arthritis from a tissue sample comprising:
obtaining a gene expression profile from the tissue sample wherein expression of the two or more of the following genes is measured:

| | | |
|---|---|---|
| 213415_at | CLIC2* | chloride intracellular channel 2 |
| 225352_at | TLOC1* | translocation protein 1 |
| 225394_s_at | MADP-1* | MADP-1 protein |
| | | Clone A9A2BRB5 (CAC)n/(GTG)n |
| | | repeat-containing |
| 211994_at | —* | mRNA |
| 212055_at | C18orf10* | chromosome 18 open reading frame 10 |
| 212174_at | AK2* | adenylate kinase 2 |
| | | similar to junction-mediating and |
| | | regulatory protein p300 |
| 228953_at | KIAA1971* | JMY |
| 230546_at | KIAA1036* | KIAA1036 |
| 230747_s_at | —* | CDNA clone IMAGE: 3029742, partial |
| | | cds |
| 242300_at | —* | — |
| 228622_s_at | DNAJC4* | DnaJ (Hsp40) homolog, subfamily C, |
| | | member 4 |
| 226296_s_at | MRPS15* | mitochondrial ribosomal protein S15 |

as compared to a normal control sample.

17. The method of claim 16, wherein the tissue comprises a leukocyte.

18. A computer readable medium comprising computer-executable instructions for performing the method for determining the genotype of a sample comprising:
obtaining a plurality of sample probe intensities;
diagnosing systemic onset juvenile idiopathic arthritis based upon the sample probe intensities for heme synthesis (delta hemoglobin or erythroid associated factor), erythrocyte-specific transcription factors (Kruppel-like factor 1), cytoskeleton (myosin light polypeptide 4), ubiquitin ligase (makorin 1), IL-1 receptor antagonist (IL-1RN), Fc Epsilon receptor, Foxo3a or GATA-3; and
calculating a linear correlation coefficient between the sample probe intensities and reference probe intensities; and accepting the tentative genotype as the genotype of the sample if the linear correlation coefficient is greater than a threshold value.

19. The method of claim 16, wherein the threshold value is at least 0.8.

20. The method of claim 16, wherein the threshold value is at least 0.9.

21. The method of claim 16, wherein the threshold value is at least 0.95.

22. The method of claim 16, wherein the probe intensities are selected from a gene expression profile from the tissue sample wherein expression of the two or more of the following genes is measured:

| | | |
|---|---|---|
| 213415_at | CLIC2* | chloride intracellular channel 2 |
| 225352_at | TLOC1* | translocation protein 1 |
| 225394_s_at | MADP-1* | MADP-1 protein |
| 211994_at | —* | Clone A9A2BRB5 (CAC)n/(GTG)n |
| | | repeat-containing mRNA |
| 212055_at | C18orf10* | chromosome 18 open reading frame 10 |
| 212174_at | AK2* | adenylate kinase 2 |
| | | similar to junction-mediating and |
| | | regulatory protein p300 |
| 228953_at | KIAA1971* | JMY |
| 230546_at | KIAA1036* | KIAA1036 |
| 230747_s_at | —* | CDNA clone IMAGE: 3029742, partial |
| | | cds |
| 242300_at | —* | — |
| 228622_s_at | DNAJC4* | DnaJ (Hsp40) homolog, subfamily C, |
| | | member 4 |
| 226296_s_at | MRPS15* | mitochondrial ribosomal protein S15 |

as compared to a normal control sample.

23. A microarray for identifying a human subject predisposed to systemic onset juvenile idiopathic arthritis comprising:
a microarray for the detection of gene expression, wherein the microarray comprises four or more biomarker selected from the group consisting of delta hemoglobin; erythroid associated factor; Kruppel-like factor 1; myosin light polypeptide 4; makorin 1, IL-1 receptor antagonist (IL-1RN), Fc Epsilon receptor, Foxo3a, and GATA-3;
wherein the gene expression data obtained from the microarray correlates to a predisposition to systemic onset juvenile idiopathic arthritis with a threshold value of at least 0.8.

24. A system for diagnosing systemic onset juvenile idiopathic arthritis comprising:
obtaining gene expression data from a microarray; and
determining the expression four or more biomarkers selected from the group consisting of delta hemoglobin; erythroid associated factor; Kruppel-like factor 1; myosin light polypeptide 4; makorin 1, IL-1 receptor antagonist (IL-1RN), Fc Epsilon receptor, Foxo3a, and GATA-3;
wherein the gene expression data obtained from the microarray correlates to a predisposition to systemic onset juvenile idiopathic arthritis with a threshold value of at least 0.8.

25. A method for diagnosing systemic onset juvenile idiopathic arthritis from a tissue sample comprising:
obtaining a gene expression profile from the tissue sample wherein expression of the two or more of the following genes is measured:

| | | | | |
|---|---|---|---|---|
| | | | Average | |
| | | | normalized | |
| | Gene | | values in | |
| Probe Set ID | Symbol | p-value | SoJIA | Gene Title |

Microtubule/Cytoskeleton

| | | | | |
|---|---|---|---|---|
| 200703_at | DNCL1 | 2.16E−04 | 1.7 | dynein, cytoplasmic, light polypeptide 1 |
| 207490_at | TUBA4 | 3.96E−04 | 1.4 | tubulin, alpha 4 |

Extracellular matrix

| | | | | |
|---|---|---|---|---|
| 216993_s_at | COL11A2 | 0.00241 | 1.4 | collagen, type XI, alpha 2 |
| 202337_at | PMF1 | 9.06E−04 | 0.7 | polyamine-modulated factor 1 |

Ubiquitination

| | | | | |
|---|---|---|---|---|
| 200718_s_at | SKP1A | 0.00462 | 1.3 | S-phase kinase-associated protein 1A |
| | | | | (p19A) |
| 201824_at | RNF14 | 0.00301 | 2.0 | ring finger protein 14 |
| 210579_s_at | TRIM10 | 0.00835 | 1.4 | tripartite motif-containing 10 |

Transport

| | | | | |
|---|---|---|---|---|
| 201066_at | CYC1 | 5.27E−04 | 0.8 | cytochrome c-1 |
| 202125_s_at | ALS2CR3 | 5.27E−04 | 2.1 | amyotrophic lateral sclerosis 2 |
| | | | | (juvenile) chromosome region, |
| | | | | candidate 3 |
| 213415_at | CLIC2* | 1.69E−05 | 8.3 | chloride intracellular channel 2 |
| 215716_s_at | ATP2B1 | 0.00241 | 0.6 | ATPase, Ca++ transporting, plasma |
| | | | | membrane 1 |
| 218211_s_at | MLPH | 0.00462 | 1.5 | melanophilin |
| 224787_s_at | RAB18 | 6.94E−04 | 0.7 | RAB18, member RAS oncogene |
| | | | | family |
| 225352_at | TLOC1* | 1.10E−05 | 2.4 | translocation protein 1 |
| 226154_at | DNM1L | 0.00836 | 0.8 | Dynamin 1-like |
| 238066_at | RBP7 | 0.00836 | 0.8 | retinol binding protein 7, cellular |
| 244227_at | SYT6 | 0.00241 | 1.3 | synaptotagmin VI |

Apoptosis

| | | | | |
|---|---|---|---|---|
| 212373_at | FEM1B | 5.27E−04 | 0.7 | Fem-1 homolog b (C. elegans) |
| 235116_at | TRAF1 | 9.06E−04 | 1.3 | TNF receptor-associated factor 1 |

Metabolism

| | | | | |
|---|---|---|---|---|
| 209301_at | CA2 | 0.00374 | 2.6 | carbonic anhydrase II |
| 209509_s at | DPAGT1 | 0.0015 | 1.2 | dolichyl-phosphate N- |
| | | | | acetylglucosaminephosphotransferase 1 |

Transciption

| | | | | |
|---|---|---|---|---|
| 202484_s_at | MBD2 | 0.00191 | 0.7 | methyl-CpG binding domain protein 2 |
| | | | | potassium voltage-gated channel, |
| 224099_at | KCNH7 | 0.00191 | 1.5 | subfamily H (eag-related), member 7 |
| 224933_s_at | JMJD1C | 0.00374 | 0.7 | jumonji domain containing 1C |
| 225527_at | CEBPG | 0.00117 | 0.7 | CCAAT/enhancer binding protein |
| | | | | (C/EBP), gamma |
| 227685_at | TMF1 | 0.0069 | 0.8 | TATA element modulatory factor 1 |
| 228785_at | ZNF281 | 0.00241 | 0.6 | Zinc finger protein 281 |
| 235389_at | PHF20 | 0.00462 | 0.8 | PHD finger protein 20 |
| 35671_at | GTF3C1 | 2.16E−04 | 1.3 | general transcription factor IIIC, |
| | | | | polypeptide 1, alpha 220 kDa |

Nuclear mRNA splicing, via spliceosome

| | | | | |
|---|---|---|---|---|
| 223416_at | SF3B14 | 0.00241 | 0.8 | splicing factor 3B, 14 kDa subunit |
| 225394_s_at | MADP-1* | 2.62E−06 | 0.6 | MADP-1 protein |

Glysocylation

| | | | | |
|---|---|---|---|---|
| 201724_s_at | GALNT1 | 0.00462 | 0.9 | UDP-N-acetyl-alpha-D- |
| | | | | galactosamine: polypeptide N- |
| | | | | acetylgalactosaminyltransferase 1 |
| 210205_at | B3GALT4 | 5.27E−04 | 1.3 | UDP-Gal: betaGlcNAc beta 1,3- |
| | | | | galactosyltransferase, polypeptide 4 |

Phosphorylation

| | | | | |
|---|---|---|---|---|
| 211992_at | WNK1 | 5.27E−04 | 2.1 | WNK lysine deficient protein kinase 1 |
| 226979_at | MAP3K2 | 0.00567 | 0.7 | Mitogen-activated protein kinase |
| | | | | kinase kinase 2 |
| 227073_at | MAP3K2 | 0.00836 | 0.8 | Mitogen-activated protein kinase |
| | | | | kinase kinase 2 |

Protein Biosynthesis

| | | | | |
|---|---|---|---|---|
| 212225_at | SUI1 | 2.16E−04 | 0.6 | Putative translation initiation factor |
| 224302_s_at | MRPS36 | 0.00374 | 0.8 | mitochondrial ribosomal protein S36 |
| 226296_s_at | MRPS15* | 3.80E−05 | 0.6 | mitochondrial ribosomal protein S15 |

Protein folding

| | | | | |
|---|---|---|---|---|
| 201759_at | TBCD | 1.12E−04 | 2.2 | tubulin-specific chaperone d |
| 225061_at | DNAJA4 | 0.00191 | 2.4 | DnaJ (Hsp40) homolog, subfamily A, |
| | | | | member 4 |
| 228622_s_at | DNAJC4* | 3.80E−05 | 0.7 | DnaJ (Hsp40) homolog, subfamily C, |
| | | | | member 4 |

Unknown

| | | | | |
|---|---|---|---|---|
| 211994_at | —* | 2.62E−06 | 2.8 | Clone A9A2BRB5 (CAC)n/(GTG)n |
| | | | | repeat-containing mRNA |
| 212055_at | C18orf10* | 5.54E−05 | 2.0 | chromosome 18 open reading frame |
| | | | | 10 |
| 212174_at | AK2* | 8.80E−07 | 0.7 | adenylate kinase 2 |
| 212341_at | MGC21416 | 0.00836 | 1.6 | hypothetical protein MGC21416 |
| 212829_at | — | 6.94E−04 | 2.0 | CDNA FLJ13267 fis, clone |
| | | | | OVARC1000964 |
| 216739_at | — | 3.96E−04 | 1.6 | — |
| 218116_at | C9orf78 | 0.00191 | 2.1 | chromosome 9 open reading frame 78 |
| 218126_at | FLJ10579 | 9.06E−04 | 1.5 | hypothetical protein FLJ10579 |
| 218583_s_at | RP42 | 0.00462 | 1.5 | RP42 homolog |
| 218936_s_at | HSPC128 | 0.00117 | 0.6 | HSPC128 protein |
| 222309_at | C6orf62 | 0.00567 | 0.6 | Chromosome 6 open reading frame |
| | | | | 62 |
| 223112_s_at | NDUFB10 | 3.96E−04 | 0.8 | NADH dehydrogenase (ubiquinone) 1 |
| | | | | beta subcomplex, 10, 22 kDa |
| 223548_at | C1orf26 | 0.0015 | 1.4 | chromosome 1 open reading frame 26 |
| 224807_at | KIAA1533 | 0.0015 | 0.8 | KIAA1533 |
| 224915_x_at | — | 9.06E−04 | 0.7 | Similar to RPE-spondin |
| 225202_at | RHOBTB3 | 0.0069 | 1.2 | Rho-related BTB domain containing 3 |
| 225213_at | TA-PP2C | 2.16E−04 | 0.8 | T-cell activation protein phosphatase |
| | | | | 2C |
| 225819_at | TBRG1 | 0.00241 | 0.7 | transforming growth factor beta |
| | | | | regulator 1 |
| 226833_at | FLJ32499 | 0.00301 | 1.3 | hypothetical protein FLJ32499 |
| 226927_at | — | 0.00374 | 1.2 | Homo sapiens, clone |
| | | | | IMAGE: 3894337, mRNA |
| 227265_at | — | 0.00301 | 0.8 | MRNA; cDNA DKFZp686N07104 |
| 228452_at | C17orf39 | 0.00625 | 1.6 | chromosome 17 open reading frame |
| | | | | 39 |
| 228953_at | KIAA1971* | 5.54E−05 | 0.6 | similar to junction-mediating and |
| | | | | regulatory protein p300 JMY |
| 229074_at | EHD4 | 0.00117 | 0.8 | EH-domain containing 4 |
| 229653_at | FLJ10979 | 0.00836 | 1.4 | Hypothetical protein FLJ10979 |
| 230118_at | — | 2.16E−04 | 1.3 | Transcribed locus |
| | | | | similar to hypothetical protein |
| 230421_at | LOC345462 | 0.00567 | 1.2 | 9630041N07 |
| 230546_at | KIAA1036* | 7.95E−05 | 1.6 | KIAA1036 |
| 230747_s_at | —* | 3.80E−05 | 0.7 | CDNA clone IMAGE: 3029742, partial |
| | | | | cds |
| 232486_at | LRFN1 | 0.00462 | 1.4 | leucine rich repeat and fibronectin |
| | | | | type III domain containing 1 |
| | | | | CDNA FLJ13427 fis, clone |
| 232709_at | — | 0.00191 | 0.7 | PLACE1002477 |
| 233469_at | psiTPTE22 | 0.00301 | 1.3 | TPTE pseudogene |
| 234305_s_at | MLZE | 9.06E−04 | 1.4 | melanoma-derived leucine zipper, |
| | | | | extra-nuclear factor |
| 235798_at | — | 0.00117 | 0.8 | — |
| 236196_at | — | 0.0015 | 0.7 | CDNA FLJ42548 fis, clone |
| | | | | BRACE3004996 |
| 241491_at | KIAA1002 | 6.94E−04 | 1.5 | KIAA1002 protein |
| 241517_at | — | 0.00117 | 1.3 | — |
| 241817_at | FLJ43654 | 3.96E−04 | 0.7 | FLJ43654 protein |
| 242003_at | LOC157697 | 0.00301 | 0.7 | Hypothetical protein LOC157697 |
| 242300_at | —* | 2.56E−05 | 4.0 | — |
| 243109_at | MCTP2 | 2.94E−04 | 1.7 | Multiple C2-domains with two |
| | | | | transmembrane regions 2 |
| 243434_at | FLJ10874 | 0.00836 | 1.2 | Hypothetical protein FLJ10874 |
| 244092_at | ZRANB3 | 0.0015 | 1.4 | Zinc finger, RAN-binding domain |
| | | | | containing 3 |
| 244390_at | — | 0.0015 | 1.8 | Transcribed locus |
| 244728_at | LOC130063 | 0.00462 | 1.4 | hypothetical gene LOC130063 |
| 53987_at | RANBP10 | 2.94E−04 | 1.8 | RAN binding protein 10 |

as compared to a control.

26. A system for diagnosing systemic onset juvenile idiopathic arthritis comprising:
determining the expression level of four or more biomarkers selected from the group consisting of delta hemoglobin; erythroid associated factor; Kruppel-like factor 1; myosin light polypeptide 4; makorin 1, IL-1 receptor antagonist (IL-1RN), Fc Epsilon receptor, Foxo3a, and GATA-3;
wherein the expression data obtained correlates to a predisposition to systemic onset juvenile idiopathic arthritis with a threshold value of at least 0.8.

27. The system of claim 26, wherein the expression level comprises protein levels.
